(19) United States
(12) Patent Application Publication
(10) Pub. No.: US 2026/0048251 A1
(43) Pub. Date: Feb. 19, 2026

(54) METHODS AND SYSTEMS FOR IMPROVED NUCLEIC ACID DELIVERY VIA ULTRASOUND

(71) Applicant: SonoThera, Inc., South San Francisco, CA (US)

(72) Inventors: FEINSTEIN B. Steven, Highland Park, IL (US); GREENBERG Kenneth, Berkeley, CA (US); CAMPBELL Barry, San Mateo, CA (US); KRIVEGA Ivan, South San Francsico, CA (US)

(21) Appl. No.: 19/368,564

(22) Filed: Oct. 24, 2025

Related U.S. Application Data
(63) Continuation of application No. PCT/US2024/026238, filed on Apr. 25, 2024.
(60) Provisional application No. 63/462,515, filed on Apr. 27, 2023.
(60) Provisional application No. 63/462,516, filed on Apr. 27, 2023.
(60) Provisional application No. 63/516,489, filed on Jul. 28, 2023.
(60) Provisional application No. 63/516,491, filed on Jul. 28, 2023.
(60) Provisional application No. 63/537,160, filed on Sep. 7, 2023.
(60) Provisional application No. 63/625,277, filed on Jan. 25, 2024.

(51) Int. Cl. A61M 37/00; A61K 9/00; A61K 31/711; A61K 41/00; A61K 48/00; A61M 5/178

(52) U.S. Cl. CPC A61M 37/0092; A61K 9/0009; A61K 31/711; A61K 41/0028; A61K 48/0016; A61K 48/0083; A61M 5/178; A61M 2210/1071; A61M 2210/1082; A61M 2210/12

(57) ABSTRACT

Disclosed herein are sonoporation processes which permit for repeating sonoporation gene therapy treatments in a safe and effective manner to improve nucleic acid delivery and expression in a target cell. The methods include processes for multiplexing application of ultrasound in combination with administration of sonoactive agents and nucleic acid payloads to increase delivery of the nucleic acid payloads to a target cell in a subject, thereby increasing gene transfection and expression, and, in some cases, increasing the durability of gene expression following a sonoporation based gene therapy treatments. The methods disclosed herein can include providing ultrasound energy to multiple locations in a target tissue comprising the target cells, and which can be applied in multiple treatment sessions which can be repeated.

### CROSS REFERENCE TO RELATED APPLICATIONS

This application is a continuation of PCT Patent Application No. PCT/US2024/026238 which claims priority to U.S. Provisional Patent Application No. 63/462,515 filed Apr. 27, 2023, U.S. Provisional Patent Application No. 63/516,489 filed Jul. 28, 2023, U.S. Provisional Patent Application No. 63/537,160 filed Sep. 7, 2023, U.S. Provisional Patent Application No. 63/625,277 filed Jan. 25, 2024, U.S. Provisional Patent Application No. 63/462,516 filed Apr. 27, 2023, and U.S. Provisional Patent Application No. 63/516,491 filed Jul. 28, 2023, each of which is incorporated herein by reference in its entirety and for all purposes.

### BACKGROUND

Gene therapy, in which a functional copy of a gene is transfected into a cell, has been proposed as a possible method of treating genetic diseases. However, prior art methods of gene therapy using ultrasound or sonoporation suffer from significant shortcomings such as low transfection rates, insufficient gene expression, and short duration of gene expression which have prevented the clinical development and commercialization of these methodologies. There remains a need in the art for an effective gene therapy technique that can transfect a gene to a cell in an organ or a tissue in a subject in a safe, effective, and durable manner.

### SUMMARY

One possible approach to achieve safe and effective gene expression using sonoporation based gene therapies is to repeat the gene therapy treatment, as unlike viral vector based gene therapies, sonoporation based gene therapies can potentially be reapplied. However identifying suitable protocols and conditions under which sonoporation based gene therapies can be safely and effectively repeated remains a challenge, as repeating ultrasound based gene therapies may result in substantial cellular damage, inflammation, and/or death as a result of the ultrasound treatment and gene transfection process, and it has not been shown that such sonoporation processes provide safe and effective therapy treatments which would justify the risk of reapplying the sonoporation gene therapy treatment. Improved processes which provide for improved nucleic acid delivery and expression to target cells represent an improvement to the state of the art.

Disclosed herein are sonoporation processes which permit for repeating sonoporation gene therapy treatments in a safe and effective manner to improve nucleic acid delivery and expression in a target cell. The methods include processes for multiplexing application of ultrasound in combination with administration of sonoactive agents and nucleic acid payloads to increase delivery of the nucleic acid payloads to a target cell in a subject, thereby increasing gene transfection and expression, and, in some cases, increasing the durability of gene expression following a sonoporation based gene therapy treatments. The methods disclosed herein can include providing ultrasound energy to multiple locations in a target tissue comprising the target cells, and which can be applied in multiple treatment sessions which can be repeated without substantial cellular damage, inflammation, and/or death following the treatment sessions. The methods disclosed herein can include administering or readministering sonoporation treatment sessions at various intervals, applying ultrasound to multiple locations on a target organ or in a target tissue, and applying ultrasound energy at various parameters to deliver the nucleic acid payload safely and effectively. Such ultrasound parameters may include application with reduced power, reduced mechanical indexes, alternating between high and low mechanical indexes, application in short flash pulses at high mechanical indexes, and/or in short overall treatment sessions to achieve beneficial results.

Aspects disclosed herein provide a method of delivering a nucleic acid payload to a target cell(s) of a subject, the method comprising: applying a first treatment session to the subject, the first treatment session comprising: administering an amount of a first therapeutic composition comprising: i) the nucleic acid payload, and ii) a plurality of sonoactive agents to the subject; administering ultrasound energy to target cell(s) of the subject at a first location in a target tissue; and administering ultrasound energy to target cell(s) of the subject at a second location, in the target tissue, wherein the first and the second location are different; and applying a second treatment session to the subject after the first treatment session, the second treatment session comprising: administering an amount of a second therapeutic composition comprising: i) the nucleic acid payload, and ii) a plurality of sonoactive agents to the subject; administering ultrasound energy to target cell(s) of the subject at a third location in the target tissue; and administering ultrasound energy to target cell(s) of the subject at a fourth location, in the target tissue wherein the third and the fourth location are different. In some embodiments, the method includes comprising: applying a subsequent treatment session after the second treatment session to the subject, the subsequent treatment session comprising: administering an amount of the first or second therapeutic composition comprising: i) the nucleic acid payload, and ii) the plurality of sonoactive agents to the subject; administering ultrasound energy to target cell(s) of the subject at a fifth location in the target tissue; and administering ultrasound energy to target cell(s) of the subject at a sixth location a subsequent location in the target tissue, wherein the fifth and the sixth location are different. In some embodiments, the method includes, in the first treatment session, administering ultrasound energy to target cell(s) of the subject at a subsequent location in the target tissue after administering ultrasound energy to the second location. In some embodiments, the method includes, in the second treatment session, administering ultrasound energy to target cell(s) of the subject at a subsequent location in the target tissue after administering ultrasound energy to the fourth location. In some embodiments, the method includes in the subsequent treatment session, administering ultrasound energy to target cell(s) of the subject at a subsequent location in the target tissue after administering ultrasound energy to the sixth location. In some embodiments, the second treatment session is more than 6 hours after but within 10 days of the first treatment session. In some embodiments, the second treatment session is more than 21 days after the first treatment session. In some embodiments, administering of the first therapeutic composition and/or the second therapeutic composition occurs intravenously through a peripheral vein. In some embodiments, the nucleic acid payload comprises a therapeutic transgene greater than 4.7 kbp in length. In some embodiments, the therapeutic transgene comprises FVIII, COL4A5, or PKD1, GLP-1, INS, Reg3g, MafA, PDX-1, NUEROG3, NGN3, DRYK, DYRKIA, DYRKIB, Factor VIII, Factor IX, PKD2, COL4A3, COL4A4, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS, or combinations thereof. In some embodiments, administering the amount of the first therapeutic composition comprises administering to the subject, in the first treatment session, a first dose of the first therapeutic composition and a second dose of the first therapeutic composition. In some embodiments, in the first treatment session, administering ultrasound energy to the subject in the second location in the target tissue occurs during or after the administering the second dose of the first therapeutic composition. In some embodiments, administering the amount of the first therapeutic composition in the first treatment session comprises administering at least a third dose of the first therapeutic composition. In some embodiments, the method includes, in the first treatment session, administering ultrasound energy to the subject at the subsequent location during or following the administering of the third dose. In some embodiments, administering the amount of the second therapeutic composition in the second treatment session comprises administering to the subject a first dose of the second therapeutic composition and a second dose of the second therapeutic composition. In some embodiments, administering ultrasound energy, in the second treatment session, to the subject at the third location in the target tissue occurs during or after the administering the second dose of the second therapeutic composition. In some embodiments, administering the amount of the second therapeutic composition in the second treatment session comprises administering at least a third dose of the second therapeutic composition. In some embodiments, each dose of first therapeutic composition or the second therapeutic composition is administered as an intravenous injection. In some embodiments, the intravenous injection is administered over a discrete time period. In some embodiments, the discrete time period is no more than 60, 120, or 180 seconds. In some embodiments, the first location in the target tissue and the third location in the target tissue are a same location. In some embodiments, the first location in the target tissue and the third location in the target tissue are different. In some embodiments, the first location in the target tissue and the subsequent location in the target tissue are a same location, in the first treatment session. In some embodiments, the first location in the target tissue and the subsequent location in the target tissue are the different, in the first treatment session. In some embodiments, the second location in the target tissue and the fourth location in the target tissue are a same location. In some embodiments, the second location in the target tissue and the fourth location in the target tissue are different. In some embodiments, in the second treatment session, in the target tissue and the subsequent location in the target tissue are a same location, in the second treatment session. In some embodiments, in the second treatment session, the second location in the target tissue and the subsequent location in the target tissue are different. In some embodiments, the fifth location is a same location as any one of the first to fourth locations. In some embodiments, the sixth is a same location as any one of the first to fourth locations. In some embodiments, the ultrasound energy is administered at an MI of at least 1.6, 2.1, or 2.3. In some embodiments, the sonoactive agents comprise a protein stabilized shell, a lipid stabilized shell, a perflutran gas core, an SF6 gas core, or combinations thereof. In some embodiments, the ultrasound energy is administered transcutaneously. In some embodiments, the target cell(s) and/or the target tissue are in a liver. In some embodiments, the first location is a first lobe of the liver and the second location or subsequent location is a second lobe or a subsequent lobe of the liver. In some embodiments, the first lobe of the liver is a right lobe, wherein the second lobe is a left lobe, and wherein the subsequent lobe is one or both of a caudate lobe or a quadrate lobe. In some embodiments, the nucleic acid payload comprises a therapeutic transgene, wherein target cell is a hepatocyte, and wherein at least 50% of cells expressing the therapeutic transgene in the liver are hepatocytes. In some embodiments, the target cell(s) and/or the target tissue are in a kidney. In some embodiments, the first location is in a first region of the kidney and the second location is in a second region of the kidney. In some embodiments, expression of the nucleic acid payload is induced in multiple cell types in the kidney. In some embodiments, a transgene encoded by the nucleic acid payload is expressed in cells in all regions of the kidney. In some embodiments, a transgene encoded by the nucleic acid payload is expressed in cells across four non-overlapping spatial regions of the kidney, the four non-overlapping spatial regions of the kidney defining an entire kidney. In some embodiments, a distance between the first location in the target tissue and the second location or subsequent location in the target tissue is at least 25% of a maximum distance of a major axis of an organ comprising the target tissue. In some embodiments, a distance between the third location in the target tissue and the fourth location or subsequent location in the target tissue is at least 25% of a maximum distance of major axis of an organ comprising the target tissue. In some embodiments, a distance between the first location in the target tissue and the second location or subsequent location in the target tissue is at least 1, 2, or 3 cm. In some embodiments, a distance between the third location in the target tissue and the fourth location or subsequent location in the target tissue is at least 1, 2, or 3 cm. In some embodiments, administering the ultrasound energy at the second location or subsequent location in the target tissue increases microvascular perfusion of the target tissue. In some embodiments, the target tissue exhibits a cystic pathology. In some embodiments, delivery and/or expression of the nucleic acid payload to the target cell(s) in the target tissue exhibiting the cystic pathology is increased. In some embodiments, the first therapeutic composition and the second therapeutic composition are a same therapeutic composition. In some embodiments, the first therapeutic composition and the second therapeutic composition comprise different dosages of the nucleic acid payload. In some embodiments, the first therapeutic composition and the second therapeutic composition comprise different dosages of sonoactive agents, or different sonoactive agents. In some embodiments, administering ultrasound energy to the subject at the first location and at the second location comprises moving an ultrasound probe across a surface of the subject's skin from the first location to the second location. In some cases, administering the second treatment session increases delivery or expression of the nucleic acid payload by at least 25%, 50%, 75%, 100%, 250%, 300%, 350%, 400%, 450%, or 500%, as compared to a method comprising administration of the first treatment session without the second treatment session. In some cases, administering the subsequent treatment session increases delivery or expression of the nucleic acid payload by at least 100%, 250%, 300%, 350%, 400%, 450%, or 500%, as compared to a method comprising administration of the first treatment session without the second treatment session, or as compared to a method comprising administration of the first treatment session and the second treatment session without administration of the subsequent treatment session. In some cases, the first location and the second location are contiguous. In some cases, the second location and the subsequent location are contiguous. In some cases, the third location and the fourth location are contiguous. In some cases, the fourth location and the subsequent location are contiguous. In some cases, the fifth location and sixth location are contiguous. In some cases, the sixth location and the subsequent location are contiguous. In some cases, the fifth location or the sixth location are contiguous with any one of the first location to the fourth location.

Aspects disclosed herein provide a method of delivering a nucleic acid payload to a target cell of a subject comprising delivering a nucleic acid payload to a target cell of a subject comprising: providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session at least once more than 6 hours after but within 10 days after initiation of a first treatment session. Aspects disclosed herein provide a method of delivering a nucleic acid payload to a target cell of a subject comprising: providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session at least once more than 21 days after initiation of a first treatment session. Aspects disclosed herein provide a method of delivering a nucleic acid payload to a target cell of a subject comprising: providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session, wherein the repeating the treatment session increases expression of a protein encoded by the nucleic acid payload by at least 5%, 10%, 20%, 30%, 40%, 50%, 100%, 250%, 500%, 750%, 1000%, or 2000% as compared to single dose administration. Aspects disclosed herein provide a method of delivering a nucleic acid payload to a target cell of a subject comprising: providing a treatment session to the subject, wherein the treatment session comprises: administering two or more doses the nucleic acid payload to the subject; administering two or more doses of a plurality of microbubbles to the subject; and administering ultrasound energy to the subject in proximity to the target cell(s); and repeating the treatment session. In some embodiments, the two or more doses the nucleic acid payload comprise three or more doses. In some embodiments, the two or more doses the nucleic acid payload comprise three or more doses of a plurality of microbubbles. In some embodiments, the repeating the treatment session comprises reapplying the ultrasound energy to a second or subsequent location on a target organ in proximity to the target cell(s). In some embodiments, the administering the nucleic acid payload to the subject comprises administering two or more doses of the nucleic acid payload to the subject during the treatment session. In some embodiments, the administering the nucleic acid payload to the subject comprises administering three or more doses of the nucleic acid payload to the subject during the treatment session. In some embodiments, the administering the plurality of microbubbles to the subject to the subject comprises administering two or more doses of the plurality of microbubbles to the subject during the treatment session. In some embodiments, the administering the plurality of microbubbles to the subject to the subject comprises administering three or more doses of the plurality of microbubbles to the subject during the treatment session. In some embodiments, the repeating the treatment session comprises re-administering two or more doses of the nucleic acid payload to the subject during the treatment session. In some embodiments, the repeating the treatment session comprises re-administering three or more doses of the nucleic acid payload to the subject during the treatment session. In some embodiments, the repeating the treatment session comprises re-administering two or more doses of the plurality of microbubbles to the subject during the treatment session. In some embodiments, the repeating the treatment session comprises re-administering three or more doses of the plurality of microbubbles to the subject during the treatment session. In some embodiments, the repeating the treatment session increases expression of a protein encoded by the nucleic acid payload by at least 5%, 10%, 20%, 30%, 40%, 50%, 100%, 250%, 500%, 750%, 1000%, or 2000% as compared to single dose administration. In some embodiments, the repeating the treatment session comprises reapplying the ultrasound energy to a second location on a target organ in proximity to the target cell(s). In some embodiments, the repeating the treatment session comprises reapplying the ultrasound energy to a same location on a target organ in proximity to the target cell(s). In some embodiments, the nucleic acid payload is a nanoplasmid. In some embodiments, the nucleic acid payload comprises a therapeutic transgene coupled to a promoter other than CMV promoter or ubiquitin C promoter. In some embodiments, the ultrasound energy is sufficient to disrupt the microbubbles. In some embodiments, the ultrasound energy is sufficient to allow the nucleic acid payload to enter the target cell. In some embodiments, the ultrasound energy is sufficient to allow the nucleic acid payload to enter the target cell, as demonstrated by expression of the nucleic acid or amino acid sequence. In some embodiments, the ultrasound energy comprises applying ultrasound energy at a mechanical index ranging from 0.05 to 2.3. In some embodiments, the ultrasound energy comprises applying ultrasound energy at a mechanical index ranging from 0.05 to 2.1. In some embodiments, the ultrasound energy comprises applying ultrasound energy at a mechanical index ranging from 0.05 to 1.6. In some embodiments, ultrasound energy comprises applying ultrasound energy for up to 30, 60, 90, 120, 150, 180, 240, 300, or 360 seconds. In some embodiments, ultrasound energy comprises applying ultrasound energy for up to 10, 15, 20, 25, or 30 minutes. In some embodiments, the administering the ultrasound energy comprises continuously applying ultrasound energy for the duration of the treatment session. In some embodiments, the applying the ultrasound acoustic energy comprises continuously applying the ultrasound acoustic energy. In some embodiments, continuously applying the ultrasound acoustic energy comprises continuously contacting the subject with an ultrasound transducer. In some embodiments, continuously applying the ultrasound acoustic energy comprises an ultrasound transducer sending ultrasound acoustic energy or receiving reflected ultrasound acoustic energy at least 95% of a period of time in which an ultrasound transducer continuously is contacting the subject. In some embodiments, continuously applying the ultrasound acoustic energy comprises the ultrasound acoustic energy being continuously applied or reflected. In some embodiments, continuously applying the ultrasound acoustic energy comprises an ultrasound transducer continuously transmitting ultrasound acoustic energy or receiving reflected ultrasound acoustic energy. In some embodiments, continuously applying ultrasound energy comprises applying ultrasound energy at a first mechanical index for a first duration, applying ultrasound energy at a second mechanical index for a second duration, and re-applying ultrasound energy at the first mechanical index without ceasing application of ultrasound energy for the duration of the continuous application of ultrasound energy. In some embodiments, the second mechanical index is higher than the first mechanical index. In some embodiments, continuously applying ultrasound energy comprises applying ultrasound energy at a first mechanical index for a first duration, applying ultrasound energy at a second mechanical index for a second duration, and re-applying ultrasound energy at the first mechanical index without ceasing application of ultrasound energy for the duration of the treatment session. In some embodiments, the treatment session as determined by the length of continuous application of ultrasound energy is up to 30, 60, 90, 120, 150, 180, 240, 300, or 360 seconds. In some embodiments, the administering of the ultrasound energy of c. comprises administering a plurality of ultrasound flashes in which the mechanical index is increased, the plurality of ultrasound flashes administered at least 2 seconds apart from one another. In some embodiments, the administering of the ultrasound energy of c. comprises administering a plurality of ultrasound flashes in which the mechanical index is increased, with each of the plurality of ultrasound flashes is administered within 1 s to about 10 s apart from one another. In some embodiments, the administering of the ultrasound energy of c. comprises administering a plurality of ultrasound flashes in which the mechanical index is increased, with each of the plurality of ultrasound flashes administered within 5 s to about 10 s apart from one another over about 0.5 s. In some embodiments, the administering of the ultrasound energy of c. comprises administering a plurality of 10 ultrasound flashes in which the mechanical index is increased, with each of the plurality of 10 ultrasound flashes administered within 1 s to about 2 s apart from one over about 0.5 s. In some embodiments, the ultrasound flash is a change in the application of the ultrasound energy from the first mechanical index to the second mechanical index, and reapplication of the ultrasound energy at the first mechanical index from the second mechanical index within a time period. In some embodiments, the time period is less than 1 s. In some embodiments, the time period is from about 0.7 us to about 3 us (microseconds). In some embodiments, the time period is from about 0.72, 0.82, 0.72, 0.98, or 2.28 us. In some embodiments, the administering of the ultrasound energy of c. comprises administering at least 5 ultrasound flashes at a second mechanical index which is increased relative to a first mechanical index less than 10 seconds apart from one another. In some embodiments, the administering the nucleic acid payload or administering the plurality of microbubbles comprises administering intravenously through a peripheral vein. In some embodiments, the expression of the nucleic acid or amino acid sequence encoded by the nucleic acid payload is sustained at a level of at least 50% of a peak expression level of the protein or the mRNA for at least 1 week. In some embodiments, the expression of the nucleic acid or amino acid sequence encoded by the nucleic acid payload is sustained at a level of at least 50% of a peak expression level of the protein or the mRNA for at least 1 week. In some embodiments, the treatment session is repeated within 72 hours after an initial performance of a first treatment session. In some embodiments, the treatment session is repeated within 48 hours after an initial performance of a first treatment session. In some embodiments, the treatment session is repeated twice within 24 hours after performance of a first treatment session. In some embodiments, the treatment session is repeated 30 days after performance of a first treatment session. In some embodiments, the treatment session is repeated 60 days after performance of a first treatment session. In some embodiments, the treatment session is repeated 90 days after performance of a first treatment session. In some embodiments, the treatment session is repeated 120 days after performance of a first treatment session. In some embodiments, repeating the treatment session does not result in a substantial elevation of inflammatory biomarker(s). In some embodiments, repeating the treatment results in substantial elevation of inflammatory biomarker(s) is within 20% of a baseline level of the inflammatory biomarker(s). In some embodiments, the biomarker(s) markers is transaminitis, ALT, AST, IL6, or combinations thereof. In some embodiments, the nanoplasmid construct is administered at a concentration of 0.4 mg/kg body mass to 1.5 mg/kg body mass. In some embodiments, a.-c. induces expression of the payload in the target cell within 24 hours. In some embodiments, the expression of the nucleic acid payload in the target cell is maintained for at least 1, 2, 3, 4, 5, 6, 7, 8, 9, 10, 11, 12, 13, 14, 15, or 16 weeks following performance of a.-c. In some embodiments, the expression of the nucleic acid payload in the target cell is maintained at an elevated level as compared to a baseline expression level for at least 1, 2, 3, 4, 5, 6, 7, 8, 9, 10, 11, 12, 13, 14, 15, or 16 weeks following performance of a.-c. In some embodiments, the elevated level of the nucleic acid payload as compared to a baseline expression level of the is at least 10% greater than the baseline expression level. In some embodiments, the administering of the nucleic acid payload of a. is administered at dosage of about 3.5 μg/μl. In some embodiments, the microbubbles comprise a protein stabilized shell. In some embodiments, the microbubbles comprise an albumin stabilized shell. In some embodiments, the microbubbles comprise perflubron gas. In some embodiments, the microbubble is an Optison microbubble. In some embodiments, the microbubbles comprise a lipid stabilized shell. In some embodiments, a concentration of the microbubbles administered is up to about 1.2*10\hat{ }10 microbubbles/ml. In some embodiments, the microbubbles are administered at a concentration of at least 5*10\hat{ }8 microbubbles/ml. In some embodiments, a concentration of the microbubbles administered is between 5*10\hat{ }8 and 8*10\hat{ }8 microbubbles/ml. In some embodiments, the administering ultrasound energy occurs transcutaneously. In some embodiments, the target cell(s) comprise hepatic cell(s). In some embodiments, the target cell(s) comprise renal cell(s). In some embodiments, the target cell(s) comprise pancreatic cell(s). In some embodiments, the target cell(s) comprise cardiac cell(s), or endothelial cell(s). In some embodiments, the target cell(s) comprise myocyte cell(s), skeletal muscle cell(s), or smooth muscle cell(s). In some embodiments, the nucleic acid payload comprises a therapeutic transgene. In some embodiments, the therapeutic transgene comprises: GLP-1, INS, Reg3g, MafA, PDX-1, NUEROG3, NGN3, DRYK, DYRKIA, DYRKIB, Factor VIII (FVIII), Factor IX (FIX), PKD1, PKD2, COL4A3, COL4A4, COL4A5, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS or combinations thereof. In some embodiments, the nucleic acid payload comprises a therapeutic transgene coupled to a promoter sequence comprising: ApoE promoter, CAG promoter, AAT promoter, or combinations thereof. In some embodiments, the nucleic acid payload and the plurality of microbubbles are administered in a volumetric ratio of at least 1 part nucleic acid payload solution to 4 parts microbubble solution, wherein there are at least 4 parts microbubble solution to nucleic acid payload solution.

In some aspects, provided herein is a method of increasing expression of a nucleic acid or amino acid sequence encoded by a payload in a target tissue of a subject, comprising administering intravenously through a peripheral vein a first dose of i) a payload, and ii) a plurality of sonoactive microstructures to the subject; administering ultrasound energy transcutaneously to the subject in proximity to target cell(s) at a first location in the target tissue; and administering ultrasound energy transcutaneously to the subject in proximity to target cell(s) at a second location in the target tissue. In some embodiments, the method further comprises administering a second dose of i) the payload, and ii) the plurality of sonoactive microstructures to the subject. In some embodiments, the method further comprises administering ultrasound energy transcutaneously to the subject in proximity to target cell(s) at a third location in the target tissue. In some embodiments, the method further comprises administering intravenously through a peripheral vein a third dose of i) the payload, and ii) the plurality of sonoactive microstructures to the subject. In some embodiments, any of the first, the second, or optionally the third dose are administered over a first time period. In some embodiments, the first time period is up to 15, 30, 45, or 60 seconds. In some embodiments, the first, the second, and optionally the third dose are administered within 6 hours of one another. In some embodiments, the first, the second, and optionally the third dose are administered within 1 hour of one another.

In some embodiments, the method increases expression of the nucleic acid or amino acid sequence encoded by the payload by at least 5%, 10%, 20%, 30%, 40%, 50%, or more, after 24 hours as compared to administration of a single dose. In some embodiments, the method comprises administering an effective amount of ultrasound energy. In some embodiments, the effective amount of ultrasound energy is sufficient to disrupt the sonoactive microstructures. In some embodiments, the effective amount of ultrasound energy is sufficient to allow the nucleic acid payload to enter the target cell. In some embodiments, the effective amount of ultrasound energy is sufficient to allow the nucleic acid payload to enter the target cell, as demonstrated by expression of the nucleic acid or amino acid sequence. In some embodiments, the effective amount of ultrasound energy comprises applying ultrasound energy at a mechanical index ranging from 0.05 to 1.8. In some embodiments, the effective amount of ultrasound energy comprises applying ultrasound energy for up to 30, 60, 90, 120, 150, 180, 240, 300, or 360 seconds. In some embodiments, the administering ultrasound energy comprises continuously applying ultrasound energy for the duration of an administration period. In some embodiments, continuously applying ultrasound energy comprises applying ultrasound energy at a first mechanical index for a first duration, applying ultrasound energy at a second mechanical index for a second duration, and re-applying ultrasound energy at the first mechanical index without ceasing application of ultrasound energy for the duration of the continuous application of ultrasound energy. In some embodiments, administering the effective amount of ultrasound energy comprises administering a plurality of ultrasound flashes several seconds apart from one another. In some embodiments, administering the effective amount of ultrasound energy comprises administering a plurality of ultrasound flashes several seconds apart from one another. In some embodiments, the plurality of ultrasound flashes are administered one or more milliseconds apart from one another. In some embodiments, wherein the plurality of ultrasound flashes are administered one or more seconds apart from one another. In some embodiments, the administering ultrasound energy comprises administering a plurality of ultrasound flashes in which the mechanical index is increased, with each of the plurality of ultrasound flashes administered within 0.9 us to about 2.5 us apart from one another. In some embodiments, the administering ultrasound energy comprises administering a plurality of ultrasound flashes in which the mechanical index is increased, with each of the plurality of ultrasound flashes administered within 0.9 us to about 2.5 us apart from one another over about 0.5 s. In some embodiments, the administering ultrasound energy of comprises administering a plurality of 10 ultrasound flashes in which the mechanical index is increased, with each of the plurality of 10 ultrasound flashes administered within 2.2 us apart from one another over about 0.5 s. In some embodiments, the ultrasound flash is a change in the application of the ultrasound energy from the first mechanical index to the second mechanical index, and reapplication of the ultrasound energy at the first mechanical index from the second mechanical index within a time period. In some embodiments, administering the effective amount of ultrasound energy comprises administering at least 5 ultrasound flashes at a second mechanical index which is increased relative to a first mechanical index less than 10 seconds apart from one another. In some embodiments, the expression of the nucleic acid payload in the target tissue is maintained for at least 5 days as compared to a baseline expression level. In some embodiments, the expression of the nucleic caid payload in the target tissue is maintained for at least 7 days as compared to a baseline expression level. In some embodiments, the expression of the nucleic acid payload in the target tissue is maintained for at least 2 weeks. In some embodiments, the target tissue is an organ. In some embodiments, the organ is a liver. In some embodiments, the first location is a first lobe of the liver and the second location is a second lobe of the liver. In some embodiments, the first and the second location are different locations of the same lobe of the liver. In some embodiments, the organ is a kidney. In some embodiments, the first location is in a first region of the kidney and the second location is in a second region of the kidney. In some embodiments, the first and the second location are different locations of the same region of the kidney. In some embodiments, the organ is a pancreas. In some embodiments, the first location is in a first region of the pancreas and the second location is in a second region of the pancreas. In some embodiments, the first and the second location are different locations of the same region of the pancreas. In some embodiments, the organ is a heart. In some embodiments, the organ is a brain. In some embodiments, the target tissue is skeletal muscle. In some embodiments, the target tissue is skeletal muscle. In some embodiments, the nucleic acid payload is a nanoplasmid.

In some embodiments, the nucleic acid payload is a transgene. In some embodiments, the nucleic acid payload comprises a therapeutic transgene. In some embodiments, increasing expression of the nucleic acid encoded by a payload comprises inducing expression of the therapeutic transgene. In some embodiments, the transgene comprises a detectible marker. In some embodiments, the detectable comprises luciferase. In some embodiments, the therapeutic transgene comprises GLP-1, INS, Reg3g, MafA, PDX-1, NUEROG3, NGN3, DRYK, DYRKIA, DYRKIB, Factor VIII, Factor IX, PKD1, PKD2, COL4A3, COL4A4, COL4A5, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS, PKD1, PKD2, COL4A3, COL4A4, COL4A5, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS, or combinations thereof. In some embodiments, the payload comprises a therapeutic transgene operably linked to a promoter sequence. In some embodiments, the promoter sequence is a promoter sequence other than a CMV promoter or a ubiquitin C promoter. In some embodiments, the promoter sequence comprises an ApoE promoter, a CAG promoter, an AAT promotor, or combinations thereof. In some embodiments, the microbubble comprises a protein stabilized shell. In some embodiments, the protein stabilized shell comprises albumin. In some embodiments, the microbubble comprises perflutren gas. In some embodiments, the sonoactive microstructures are administered at a concentration of at least 5*10\hat{ }8 sonoactive microstructures/mL. In some embodiments, the sonoactive microstructures are administered at a concentration between about 5*10\hat{ }8 and about 8*10\hat{ }8 sonoactive microstructures/mL. In some embodiments, wherein the plurality of sonoactive microstructures comprise Optison sonoactive microstructures. In some embodiments, the sonoactive microstructures comprise a lipid stabilized shell. In some embodiments, a concentration of the sonoactive microstructures administered is up to about 1.2*10\hat{ }10 sonoactive microstructures/mL. In some embodiments, the method does not result in a substantial elevation of inflammatory biomarker(s). In some embodiments, a level of an inflammatory biomarker(s) remains within 20% of a baseline level of the inflammatory biomarker(s) after administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue. In some embodiments, the biomarker(s) is one or more of ALT, AST, and/or IL6. In some embodiments, the nucleic acid payload and the plurality of sonoactive microstructures are administered in a volumetric ratio of at least 1 part nucleic acid payload solution to 4 parts microbubble solution. In some aspects, provided herein are methods of delivering a payload into one or more target cells, the method comprising: administering intravenously through a peripheral vein a first dose of i) a payload comprising a therapeutic transgene, and ii) a plurality of sonoactive microstructures to the subject; administering ultrasound energy transcutaneously to the subject in proximity to target cell(s) at a first location in the target tissue; and administering ultrasound energy transcutaneously to the subject in proximity to target cell(s) at a second location in the target tissue. In some embodiments, the therapeutic transgene comprises a sequence encoding FVIII. In some embodiments, the therapeutic transgene comprises a sequence encoding COL4A3. In some embodiments, the therapeutic transgene comprises a sequence encoding COL4A4. In some embodiments, the therapeutic transgene comprises a sequence encoding COL4A5. In some embodiments, the therapeutic transgene comprises a sequence encoding PKD1. In some the therapeutic transgene comprises a sequence encoding PKD2. In some embodiments, administering the ultrasound energy proximal to the sonoactive microstructures increases microvascular perfusion of a target tissue. In some embodiments, administering the ultrasound energy at the second location in the target tissue increases microvascular perfusion of a target tissue. In some embodiments, repeating the sonoporation treatment increases microvascular perfusion of a target tissue. In some embodiments, the target cell is in a target tissue exhibiting cystic pathology. In some embodiments, the target cell is in a target tissue exhibiting cystic pathology, wherein delivery of the nucleic acid to the target cell in the tissue exhibiting cystic pathology is increased. In some embodiments, target cell is comprised by a subject having polycystic kidney disease, wherein the therapeutic transgene comprises a sequence encoding PKD1 or PDK2, wherein delivery of the therapeutic transgene to the target cell is increased, thereby treating polycystic kidney disease.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates experimental protocols for experiments in which sonoporation gene therapy treatments were repeated in a subject;

FIG. 2 provides fluorescence images collected by IVIS fluorescence imaging for experiments in which sonoporation gene therapy treatments were repeated in a subject;

FIG. 3A provides bar plots of the average fluorescence radiance measurement values for each group of subjects illustrated in FIG. 2;

FIG. 3B provides a line plot of average fluorescence radiance measurement values for each group of subjects illustrated in FIG. 2, in which it is shown that repeating the sonoporation gene therapy treatments provides over an order of magnitude increase as compared to a single dose;

FIG. 4 illustrates the percent change in measured average fluorescence radiance from the 24 h after first dose measurement in which sonoporation gene therapy treatments were repeated in a subject;

FIG. 5 illustrates experimental protocols for experiments in which sonoporation gene therapy treatments were repeated in a subject;

FIG. 6 provides fluorescence images collected by IVIS fluorescence imaging for experiments in which sonoporation gene therapy treatments were repeated in a subject;

FIG. 7A provides bar plots of average fluorescence radiance measurement values collected by IVIS fluorescence imaging in which sonoporation gene therapy treatments were repeated in a subject;

FIG. 7B provides bar plots of the average fluorescence radiance measurement values for subjects illustrated in FIG. 6;

FIG. 7C illustrates the average fluorescence radiance measurement values for subjects illustrated in FIG. 6, in which repeated sonoporation gene therapy treatments resulted in an order of magnitude increase in measured fluorescence radiance;

FIG. 8 shows ALT, IL6 and AST serum levels measured from subjects 24 hours following a sonoporation treatment, and illustrate that there was no elevation of ALT, IL6 and AST serum levels as a result of the sonoporation treatment;

FIG. 9A illustrates an embodiment of a sonoporation treatment at the cellular level;

FIG. 9B illustrates an embodiment of a sonoporation treatment at the cellular level;

FIG. 10 provides bar plots of average fluorescence radiance measurement values in which sonoporation gene therapy treatments were repeated in a subject in different target organs

FIG. 11 illustrates experimental protocols for experiments in which sonoporation gene therapy treatments were repeated in a subject 16 weeks after the initial treatment;

FIG. 12A provides bar plots of average fluorescence radiance measurement values collected by IVIS fluorescence imaging for experiments in which sonoporation gene therapy treatments were repeated in a subject 16 weeks after the initial treatment;

FIG. 12B provides bar plots of average fluorescence radiance measurement values collected by IVIS fluorescence imaging for experiments in which sonoporation gene therapy treatments were repeated 16 weeks after the initial treatment in two groups of mice. Mice in one group received a smaller dose of DNA (light gray bars) and mice in the other group received a larger dose of DNA (dark gray bars, also shown in FIG. 12A);

FIG. 13 shows average fluorescence radiance measurement values collected by IVIS fluorescence imaging for experiments testing the efficacy of different doses of DNA delivered during sonoporation treatments of the liver;

FIG. 14 shows average fluorescence radiance measurement values collected by IVIS fluorescence imaging for experiments testing the efficacy of different doses of DNA delivered during sonoporation treatments of the kidney;

FIG. 15 shows average fluorescence radiance measurement values collected by IVIS fluorescence imaging for experiments testing the efficacy of repeated doses of DNA delivered during sonoporation treatments of the kidney;

FIG. 16 illustrates an exemplary experimental protocol in which one, two, or three doses (“boluses”) of a DNA construct and sonoactive microstructures (“sonoactive microstructures”) are delivered to a subject in conjunction with ultrasound delivered at one, two, or three locations in a target tissue;

FIG. 17 provides fluorescence images collected by IVIS fluorescence imaging in a multi-dose, multi-location experiment such as the experiment described in FIG. 16, and illustrates that subjects receiving two, or three doses (“boluses”) of a DNA construct and sonoactive microstructures exhibited increased gene expression, and increased durability of gene expression in the weeks following treatment;

FIG. 18 provides fluorescence radiance measurement values (p/s/cm²/sr) for each group of subjects shown in FIG. 17 collected by IVIS fluorescence imaging at the indicated time points, and illustrates that subjects receiving two, or three doses (“boluses”) of a DNA construct and sonoactive microstructures exhibited increased gene expression, and increased durability of gene expression in the weeks following treatment;

FIG. 19 provides fluorescence radiance measurement values (p/s/cm²/sr) for four groups of subjects in a renal sonoporation transfection study. Compared to control mice, the highest average fluorescence radiance was measured in a mouse model of polycystic kidney disease (Nek^{8ick}) that received ultrasound mediated gene delivery (UMGD), indicating that UMGD in Nek^{8ick} mice resulted in microvascular perfusion, efficient gene delivery, and robust expression in cystic structures;

FIG. 20 provides 52 week fluorescence radiance data (p/s/cm²/sr) for subjects undergoing a sonoporation treatment in the liver using the methods described herein;

FIG. 21 provides 52 week fluorescence radiance data (p/s/cm²/sr) for subjects undergoing a sonoporation treatment in the kidney using the methods described herein;

FIG. 22 provides 26 week fluorescence radiance data (p/s/cm²/sr) for subjects undergoing a sonoporation treatment at various nucleic acid payload dosages using the methods described herein;

FIG. 23A provides histological images showing expression of an enhanced green fluorescent protein (EGFP) genetic payload in up to approximately 70% of glomeruli in a non-human primate (NHP) kidney;

FIG. 23B provides quantitation of expression of an enhanced green fluorescent protein (EGFP) genetic payload in glomeruli of the NHP kidney;

FIG. 24 provides a histological image showing expression of an enhanced green fluorescent protein (EGFP) genetic payload in renal cells, including podocytes, tubular epithelial cells and endothelial cells, in an NHP;

FIG. 25A provides a histological image showing expression of an enhanced green fluorescent protein (EGFP) genetic payload in renal cells in non-glomeruli areas, including podocytes, tubular epithelial cells and endothelial cells, in an NHP kidney;

FIG. 25B provides quantitation of expression of an enhanced green fluorescent protein (EGFP) genetic payload in NHP renal cells in non-glomeruli areas, including podocytes, tubular epithelial cells (Tubl) and endothelial cells (Endo);

FIG. 26A provides a histological image showing expression of an enhanced green fluorescent protein (EGFP) genetic payload in renal cells in glomeruli areas, including podocytes, tubular epithelial cells and endothelial cells, in an NHP kidney;

FIG. 26B provides quantitation of expression of an enhanced green fluorescent protein (EGFP) genetic payload in NHP renal cells in glomeruli areas, including podocytes, tubular epithelial cells (Tubl) and endothelial cells (Endo);

FIG. 27 provides a histological image showing expression of an enhanced green fluorescent protein (EGFP) genetic payload in renal cells, including podocytes, tubular epithelial cells (Tub. Cells) and endothelial cells (Endo. cell), in a murine;

FIG. 28A provides a histological image showing expression of an enhanced green fluorescent protein (EGFP) genetic payload in renal cells in non-glomeruli areas, including podocytes, tubular epithelial cells and endothelial cells, in a murine kidney;

FIG. 28B provides quantitation of expression of an enhanced green fluorescent protein genetic payload (EGFP) in murine renal cells in non-glomeruli areas, including podocytes, tubular epithelial cells (Tubl) and endothelial cells (Endo);

FIG. 29A provides a histological image showing expression of an enhanced green fluorescent protein (EGFP) genetic payload in renal cells in glomeruli areas, including podocytes, tubular epithelial cells and endothelial cells, in a murine kidney;

FIG. 29B provides quantitation of expression of and enhanced green fluorescent protein (EGFP) genetic payload in murine renal cells in glomeruli areas, including podocytes, tubular epithelial cells (Tubl) and endothelial cells (Endo);

FIG. 30A provides the distribution of kidney cell types (podocytes, tubular epithelial cells, endothelial cells, and unknown) in a murine kidney sample;

FIG. 30B provides quantification of the percentage of kidney cells of different types (podocytes, tubular epithelial cells, endothelial cells, and unknown) identified by snRNA-seq (left) and RNAscope (right) in murine kidney samples;

FIG. 31A provides a histological image showing expression of an enhanced green fluorescent protein (EGFP) genetic payload in cells in a murine liver, including hepatocytes, Kupffer/immune cells, and liver sinusoidal endothelial cells (LSECs);

FIG. 31B provides quantitation of expression of enhanced green fluorescent protein (EGFP) in murine liver cells including hepatocytes, Kupffer/immune cells, and liver sinusoidal endothelial cells (LSECs);

FIG. 32A provides the distribution of liver cell types (hepatocytes, Kupffer/immune cells, liver sinusoidal endothelial cells (LSECs), and unknown) in a murine liver sample;

FIG. 32B provides quantification of the percentage of hepatic cells of different types (hepatocytes, Kupffer/immune cells, liver sinusoidal endothelial cells (LSECs), and unknown) identified by snRNA-seq (left) and RNAscope (right) in murine liver samples;

FIG. 33 shows immunofluorescence staining for luciferase performed on sonoporated kidney sections;

FIG. 34A shows data illustrating gene delivery and gene expression in the murine liver utilizing the sonoporation treatment protocols described herein; and

FIG. 34B shows data illustrating gene delivery and gene expression in the murine liver utilizing the sonoporation treatment protocols described herein.

### DETAILED DESCRIPTION

One possible approach to achieve safe and effective gene expression using sonoporation based gene therapies is to repeat the gene therapy treatment, as unlike viral vector based gene therapies, sonoporation based gene therapies can potentially be reapplied. However identifying suitable protocols and conditions under which sonoporation based gene therapies can be safely and effectively repeated remains a challenge, as repeating ultrasound based gene therapies may result in substantial cellular damage, inflammation, and/or death as a result of the ultrasound treatment and gene transfection process, and it has not been shown that such sonoporation processes provide safe and effective therapy treatments which would justify the risk of reapplying the sonoporation gene therapy treatment. Improved processes which provide for improved nucleic acid delivery and expression to target cells represent an improvement to the state of the art.

Disclosed herein are sonoporation processes which permit for repeating sonoporation gene therapy treatments in a safe and effective manner to improve nucleic acid delivery and expression in a target cell. The methods include processes for multiplexing application of ultrasound in combination with administration of sonoactive agents and nucleic acid payloads to increase delivery of the nucleic acid payloads to a target cell in a subject, thereby increasing gene transfection and expression, and, in some cases, increasing the durability of gene expression following a sonoporation based gene therapy treatments. The methods disclosed herein can include providing ultrasound energy to multiple locations in a target tissue comprising the target cells, and which can be applied in multiple treatment sessions which can be repeated without substantial cellular damage, inflammation, and/or death following the treatment sessions. The methods disclosed herein can include administering or readministering sonoporation treatment sessions at various intervals, applying ultrasound to multiple locations on a target organ or in a target tissue, and applying ultrasound energy at various parameters to deliver the nucleic acid payload safely and effectively. Such ultrasound parameters may include application with reduced power, reduced mechanical indexes, alternating between high and low mechanical indexes, application in short flash pulses at high mechanical indexes, and/or in short overall treatment sessions to achieve beneficial results.

Aspects disclosed herein provide a method of delivering a nucleic acid payload to a target cell(s) of a subject, the method comprising: applying a first treatment session to the subject, the first treatment session comprising: administering an amount of a first therapeutic composition comprising: i) the nucleic acid payload, and ii) a plurality of sonoactive agents to the subject; administering ultrasound energy to target cell(s) of the subject at a first location in a target tissue; and administering ultrasound energy to target cell(s) of the subject at a second location, in the target tissue, wherein the first and the second location are different; and applying a second treatment session to the subject after the first treatment session, the second treatment session comprising: administering an amount of a second therapeutic composition comprising: i) the nucleic acid payload, and ii) a plurality of sonoactive agents to the subject; administering ultrasound energy to target cell(s) of the subject at a third location in the target tissue; and administering ultrasound energy to target cell(s) of the subject at a fourth location, in the target tissue wherein the third and the fourth location are different. In some embodiments, the method includes comprising: applying a subsequent treatment session after the second treatment session to the subject, the subsequent treatment session comprising: administering an amount of the first or second therapeutic composition comprising: i) the nucleic acid payload, and ii) the plurality of sonoactive agents to the subject; administering ultrasound energy to target cell(s) of the subject at a fifth location in the target tissue; and administering ultrasound energy to target cell(s) of the subject at a sixth location a subsequent location in the target tissue, wherein the fifth and the sixth location are different. In some embodiments, the method includes, in the first treatment session, administering ultrasound energy to target cell(s) of the subject at a subsequent location in the target tissue after administering ultrasound energy to the second location. In some embodiments, the method includes, in the second treatment session, administering ultrasound energy to target cell(s) of the subject at a subsequent location in the target tissue after administering ultrasound energy to the fourth location. In some cases, administering the second treatment session increases delivery or expression of the nucleic acid payload by at least 25%, 50%, 75%, 100%, 250%, 300%, 350%, 400%, 450%, or 500%, as compared to a method comprising administration of the first treatment session without the second treatment session. In some cases, administering the subsequent treatment session increases delivery or expression of the nucleic acid payload by at least 100%, 250%, 300%, 350%, 400%, 450%, or 500%, as compared to a method comprising administration of the first treatment session without the second treatment session, or as compared to a method comprising administration of the first treatment session and the second treatment session without administration of the subsequent treatment session. In some cases, the first location and the second location are contiguous. In some cases, the second location and the subsequent location are contiguous. In some cases, the third location and the fourth location are contiguous. In some cases, the fourth location and the subsequent location are contiguous. In some cases, the fifth location and sixth location are contiguous. In some cases, the sixth location and the subsequent location are contiguous. In some cases, the fifth location or the sixth location are contiguous with any one of the first location to the fourth location. Provided herein are methods for nucleic acid transfection into and expression in a cell, tissue, or organ of a subject in a targeted manner using sonoporation (e.g., a process comprising applying an ultrasonic acoustic energy to a cell, tissue, or organ, such as to provide increased porosity in the cell, tissue, or organ). As used herein, in some embodiments, a subject herein is a mammal. In some embodiments, the mammal is, by way of non-limiting example, a human, a monkey, or another non-human primate. In some embodiments, the subject may be a rat, a mouse, or another non-primate animal.

Provided in certain embodiments herein are methods for transfecting a nucleic acid construct into a target cell or tissue (e.g., of a subject) by applying a sonoporation treatment session to the subject (e.g., ultrasound mediated gene delivery), the treatment session comprising: administering the nucleic acid payload to the subject.

Provided in certain embodiments herein are methods for transfecting a nucleic acid construct into a target cell or tissue (e.g., of a subject) by applying a sonoporation treatment session to the subject, the treatment session comprising: administering a plurality of microbubbles to the subject.

Provided in certain embodiments herein are methods for transfecting a nucleic acid construct into a target cell or tissue (e.g., of a subject) by applying a sonoporation treatment session to the subject, the treatment session comprising: administering ultrasound energy to the subject in proximity to the target cell(s).

In some embodiments, a method provided herein comprises repeating the treatment session.

In some embodiments, a method provided herein comprises repeating the treatment session at least once more than 6 hours after but within 10 days after initiation of a first treatment session.

In some embodiments, a method provided herein comprises repeating the treatment session at least once more than 21 days after initiation of a first treatment session.

In some embodiments, a method provided herein comprises repeating the treatment session, wherein the repeating the treatment session increases expression of a protein or amino acid sequence encoded by the nucleic acid payload by at least 5%, 10%, 20%, 30%, 40%, 50%, 100%, 250%, 500%, 750%, 1000%, or 2000% as compared to single dose administration.

In embodiments, repeating the treatment session increases expression of a protein encoded by the nucleic acid payload by at least 5% as compared to single dose administration. In embodiments, repeating the treatment session increases expression of a protein encoded by the nucleic acid payload by at least 10% as compared to single dose administration. In embodiments, repeating the treatment session increases expression of a protein encoded by the nucleic acid payload by at least 20% as compared to single dose administration. In embodiments, repeating the treatment session increases expression of a protein encoded by the nucleic acid payload by at least 30% as compared to single dose administration. In embodiments, repeating the treatment session increases expression of a protein encoded by the nucleic acid payload by at least 40% as compared to single dose administration. In embodiments, repeating the treatment session increases expression of a protein encoded by the nucleic acid payload by at least 50% as compared to single dose administration. In embodiments, repeating the treatment session increases expression of a protein encoded by the nucleic acid payload by at least 100% as compared to single dose administration. In embodiments, repeating the treatment session increases expression of a protein encoded by the nucleic acid payload by at least 250% as compared to single dose administration. In embodiments, repeating the treatment session increases expression of a protein encoded by the nucleic acid payload by at least 500% as compared to single dose administration. In embodiments, repeating the treatment session increases expression of a protein encoded by the nucleic acid payload by at least 750% as compared to single dose administration. In embodiments, repeating the treatment session increases expression of a protein encoded by the nucleic acid payload by at least 1000% as compared to single dose administration. In embodiments, repeating the treatment session increases expression of a protein encoded by the nucleic acid payload by at least 2000% as compared to single dose administration.

In some embodiments, the treatment session is repeated at least once more than 6 hours after but within 10 days after initiation of a first treatment session. In some embodiments, the treatment session is repeated at least once more than 21 days after initiation of a first treatment session. In some embodiments, repeating the treatment session comprises reapplying ultrasound energy to a second location on a target organ in proximity to the target cell(s). In some embodiments, repeating the treatment session comprises reapplying the ultrasound energy to a same location on a target organ in proximity to the target cell(s). In some embodiments, expression of the nucleic acid or amino acid sequence encoded by the nucleic acid payload is sustained at a level of at least 50% of a peak expression level for at least 1 week. In some embodiments, expression of a nucleic acid or amino acid sequence encoded by a nucleic acid payload is sustained at a level of at least 50% of a peak expression level of the protein or the mRNA for at least 1 week. In some embodiments, repeating the treatment session sustains production of a protein or mRNA encoded by the nucleic acid payload between subsequent treatment sessions within 35% of a peak expression level.

In some embodiments, the treatment session is repeated at least once more than 6 hours after but within 10 days after initiation of a first treatment session. In some embodiments, the treatment session is repeated at least once more than 21 days after initiation of a first treatment session. In some embodiments, repeating the treatment session comprises administering ultrasound energy to a third and a fourth location in the target tissue. In some embodiments, repeating the treatment session comprises a subsequent treatment session comprising administering ultrasound energy at a fifth and a sixth location in the target tissue. In some embodiments, expression of the nucleic acid or amino acid sequence encoded by the nucleic acid payload is sustained at a level of at least 50% of a peak expression level for at least 1 week. In some embodiments, expression of a nucleic acid or amino acid sequence encoded by a nucleic acid payload is sustained at a level of at least 50% of a peak expression level of the protein or the mRNA for at least 1 week. In some embodiments, repeating the treatment session sustains production of a protein or mRNA encoded by the nucleic acid payload between subsequent treatment sessions within 35% of a peak expression level

In some embodiments, the expression of the nucleic acid payload in the target cell is maintained for at least 1, 2, 3, 4, 5, 6, 7, 8, 9, 10, 11, 12, 13, 14, 15, or 16 weeks following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue). In some embodiments, the expression of the nucleic acid payload in the target cell is maintained for at least 1 week following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue). In some embodiments, the expression of the nucleic acid payload in the target cell is maintained for at least 2 weeks following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue). In some embodiments, the expression of the nucleic acid payload in the target cell is maintained for at least 3 weeks following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue). In some embodiments, the expression of the nucleic acid payload in the target cell is maintained for at least 4 weeks following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue). In some embodiments, the expression of the nucleic acid payload in the target cell is maintained for at least 5 weeks following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue).

In some embodiments, the expression of the nucleic acid payload in the target cell is maintained at an elevated level as compared to a baseline expression level for at least 1 week following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue). In some embodiments, the expression of the nucleic acid payload in the target cell is maintained at an elevated level as compared to a baseline expression level for at least 1 week following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue).

In some embodiments, repeating the treatment session increases expression of a protein encoded by the nucleic acid payload by at least 5%, 10%, 20%, 30%, 40% or 50% as compared to single dose administration. In some embodiments, repeating the treatment session increases expression of a protein encoded by the nucleic acid payload by at least 5% as compared to single dose administration. In some embodiments, repeating the treatment session increases expression of a protein encoded by the nucleic acid payload by at least 10% as compared to single dose administration. In some embodiments, repeating the treatment session increases expression of a protein encoded by the nucleic acid payload by at least 20% as compared to single dose administration. In some embodiments, repeating the treatment session increases expression of a protein encoded by the nucleic acid payload by at least 30% as compared to single dose administration. In some embodiments, repeating the treatment session increases expression of a protein encoded by the nucleic acid payload by at least 40% as compared to single dose administration. In some embodiments, repeating the treatment session increases expression of a protein encoded by the nucleic acid payload by at least 50% as compared to single dose administration.

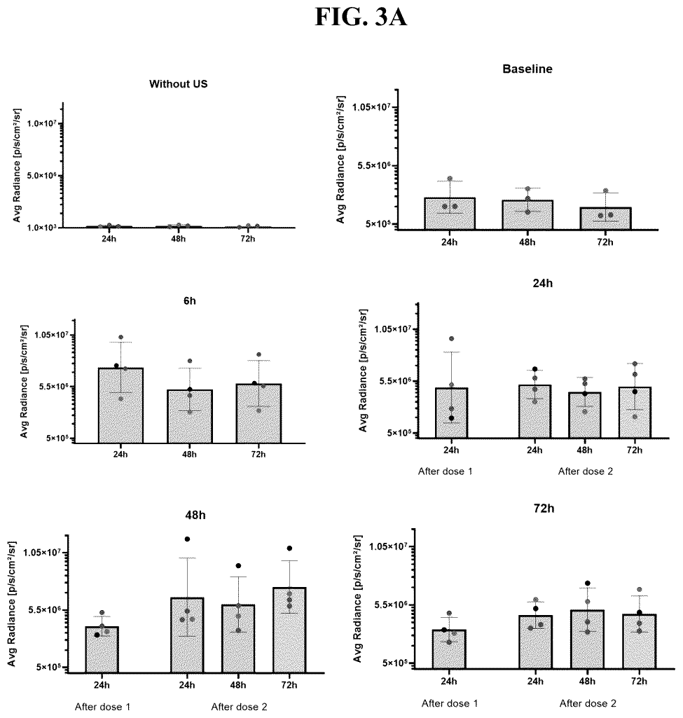

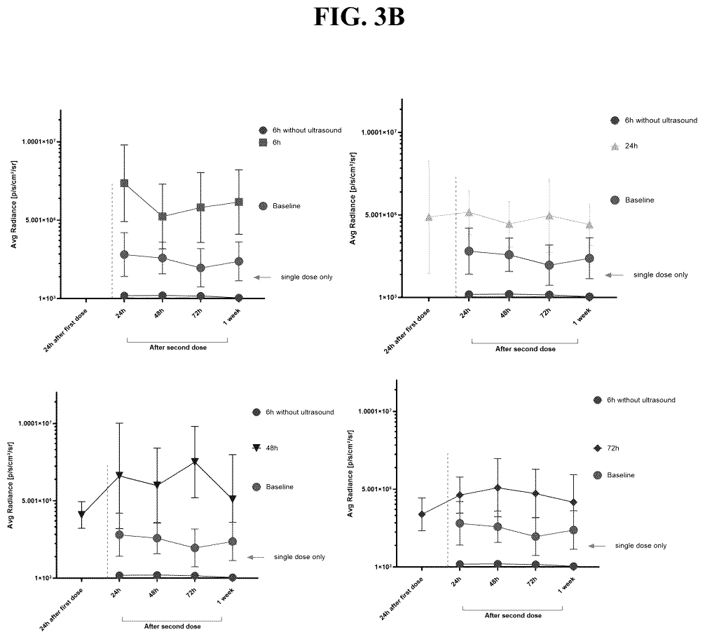

When, how often, and under what conditions sonoporation based gene therapies may be repeated as to produce improved and durable gene expression in a subject which is superior to single dose administration, and which is safe for the subject and does not result in cellular substantial inflammation, damage, and/or death, are significant factors in the ability to repeat gene sonoporation gene therapy treatments. For example, FIG. 3B illustrates that repeating sonoporation gene therapy treatments as described herein can provide over an order of magnitude difference in levels of gene expression, as compared to a single dose administration; and even among repeated sonoporation gene therapies there can be about a half order of magnitude difference in levels of gene expression depending on when the interval of the treatment sessions. Provided herein are methods comprising sonoporation gene therapy treatment protocols to produce improved and durable gene expression in a subject superior to single dose administration, which is safe and effective for the subject.

In some embodiments, the treatment session is repeated within 72 hours after the first treatment session. In some embodiments, the treatment session is repeated within 48 hours after the first treatment session. In some embodiments, the treatment session is repeated twice within 24 hours after performance of the first treatment session. In some embodiments, the treatment session is repeated at least 30 days after performance of a first treatment session. In some embodiments, the treatment session is repeated at least 30 days after performance of a first treatment session. In some embodiments, re-applying the sonoporation gene therapy treatment at the intervals described herein increases the efficacy of the treatment, nucleic acid delivery, and gene expression, without resulting in substantial cellular damage, inflammation, and/or death as a result of the ultrasound treatment and gene transfection process.

In some embodiments, a treatment session comprises administering the nucleic acid payload and sonoactive microstructures as a “bolus” over a short period of time. In some embodiments, the short period of time is less than 15 seconds. In some embodiments, the short period of time is up to 15 seconds. In some embodiments, the short period of time is up to 20 seconds. In some embodiments, the short period of time is up to 25 seconds. In some embodiments, the short period of time is up to 30 seconds. In some embodiments, the short period of time is up to 60 seconds. In some embodiments, delivering the dose of nucleic acid payload and sonoactive agents as a bolus injection immediately preceding or shortly preceding application of ultrasound to the target tissue increases the concentration of sonoactive agents and nucleic acids in the target tissue and in proximity to the target cells during sonoactive agent oscillation and prior to disruption of the sonoactive agents, thereby increasing the delivery of the nucleic acid payload to the target cells, and/or expression of the nucleic acid payload in the target cells.

In some embodiments, administering the amount of the first therapeutic composition comprises administering to the subject, in the first treatment session, a first dose of the first therapeutic composition and a second dose of the first therapeutic composition. In some embodiments, in the first treatment session, administering ultrasound energy to the subject in the second location in the target tissue occurs during or after the administering the second dose of the first therapeutic composition. In some embodiments, administering the amount of the first therapeutic composition in the first treatment session comprises administering at least a third dose of the first therapeutic composition. In some embodiments, the method includes in the first treatment session, administering ultrasound energy to the subject at the subsequent location during or following the administering of the third dose. In some embodiments, administering the amount of the second therapeutic composition in the second treatment session comprises administering to the subject a first dose of the second therapeutic composition and a second dose of the second therapeutic composition. In some embodiments, administering ultrasound energy, in the second treatment session, to the subject at the third location in the target tissue occurs during or after the administering the second dose of the second therapeutic composition. In some embodiments, administering the amount of the second therapeutic composition in the second treatment session comprises administering at least a third dose of the second therapeutic composition. In some embodiments, each dose of first therapeutic composition or the second therapeutic composition is administered as an intravenous injection. In some embodiments, the intravenous injection is administered over a discrete time period. In some embodiments, the discrete time period is no more than 60, 120, or 180 seconds.

In some embodiments, a treatment session comprises administering two, or more doses (“boluses”) of a DNA construct and sonoactive microstructures. In some embodiments, administering two, or more doses (“boluses”) of a DNA construct and sonoactive microstructures during a treatment session increases expression of a protein encoded by the nucleic acid payload by at least 5% as compared to single dose administration. In specific embodiments, administering two, or more doses (“boluses”) of a DNA construct and sonoactive microstructures during a treatment session increases expression of a protein encoded by the nucleic acid payload by at least 10% as compared to single dose administration. In specific embodiments, administering two, or more doses (“boluses”) of a DNA construct and sonoactive microstructures during a treatment session increases expression of a protein encoded by the nucleic acid payload by at least 20% as compared to single dose administration. In specific embodiments, administering two, or more doses (“boluses”) of a DNA construct and sonoactive microstructures during a treatment session increases expression of a protein encoded by the nucleic acid payload by at least 30% as compared to single dose administration. In specific embodiments, administering two, or more doses (“boluses”) of a DNA construct and sonoactive microstructures increases expression of a protein encoded by the nucleic acid payload by at least 40% as compared to single dose administration. In specific embodiments, administering two, or more doses (“boluses”) of a DNA construct and sonoactive microstructures during a treatment session increases expression of a protein encoded by the nucleic acid payload by at least 50% as compared to single dose administration. In specific embodiments, administering two, or more doses (“boluses”) of a DNA construct and sonoactive microstructures during a treatment session increases expression of a protein encoded by the nucleic acid payload by at least 100% as compared to single dose administration. In specific embodiments, administering two, or more doses (“boluses”) of a DNA construct and sonoactive microstructures during a treatment session increases expression of a protein encoded by the nucleic acid payload by at least 250% as compared to single dose administration. In specific embodiments, administering two, or more doses (“boluses”) of a DNA construct and sonoactive microstructures during a treatment session increases expression of a protein encoded by the nucleic acid payload by at least 500% as compared to single dose administration. In specific embodiments, administering two, or more doses (“boluses”) of a DNA construct and sonoactive microstructures during a treatment session increases expression of a protein encoded by the nucleic acid payload by at least 750% as compared to single dose administration. In specific embodiments, administering two, or more doses (“boluses”) of a DNA construct and sonoactive microstructures during a treatment session increases expression of a protein encoded by the nucleic acid payload by at least 1000% as compared to single dose administration. In specific embodiments, administering two, or more doses (“boluses”) of a DNA construct and sonoactive microstructures during a treatment session increases expression of a protein encoded by the nucleic acid payload by at least 2000% as compared to single dose administration.

Ultrasound protocols to be utilized in repeated sonoporation treatment sessions, which are safe for use in repeated sonoporation treatment sessions, so as to improve gene transfection and expression are significant factors in the ability to repeat gene sonoporation gene therapy treatments. Aspects disclosed herein provide methods of repeating sonoporation gene therapy treatments, without inducing substantial inflammation of the target organ, tissue, or cells; substantial cellular damage, inflammation, or death; or otherwise inducing substantial elevation of inflammatory or apoptotic cellular biomarker(s) within the subject without resulting in cellular, damage, inflammation, or death. In some embodiments, the ultrasound energy (e.g., administered to a subject) is sufficient to disrupt microbubbles. In some embodiments, the ultrasound energy (e.g., administered to a subject) is sufficient to allow a nucleic acid payload to enter a target cell. In some embodiments, the ultrasound energy (e.g., administered to a subject) is sufficient to allow a nucleic acid payload to enter a target cell, as demonstrated by expression of the nucleic acid or amino acid sequence.

In some embodiments, administering ultrasound energy can comprise continuously applying ultrasound energy for the duration of the treatment session. In some embodiments, administering ultrasound energy comprises continuously applying ultrasound energy for a portion of the duration of the treatment session. In some embodiments, ultrasound energy may be applied continuously. In some embodiments, the application of ultrasound energy comprises administering a plurality of ultrasound flashes in which the ultrasound energy is applied at a high mechanical index for a brief duration before returning to application of the ultrasound energy at the lower mechanical index. In some embodiments, these ultrasound flashes can be several seconds apart from one another. In some embodiments, continuously applying ultrasound energy comprises the ultrasound transducer continuously sending and receiving ultrasound signal.

In some more embodiments, ultrasound energy can be continuously applied by continuously alternating between ultrasound energy at a first mechanical index applied for a first duration and ultrasound energy at a second mechanical index applied for a second duration. In some more embodiments, ultrasound energy can be continuously applied by applying ultrasound energy at a first mechanical index applied for a first duration, applying ultrasound energy at a second mechanical index applied for a second duration, and re-applying ultrasound energy at the first mechanical index without ceasing application of ultrasound energy for the duration of the continuous application of ultrasound energy. In some more embodiments, ultrasound energy can be continuously applied by applying ultrasound energy at a first mechanical index applied for a first duration, applying ultrasound energy at a second mechanical index applied for a second duration, and re-applying ultrasound energy at the first mechanical index without ceasing application of ultrasound energy for the duration of the treatment session. In some embodiments, the ultrasound energy can be applied at alternating mechanical indices without ceasing application of the ultrasound energy for the duration of the continuous application of ultrasound energy. In some embodiments, the ultrasound energy can be applied at alternating mechanical indices without ceasing application of the ultrasound energy for the duration of the duration of the treatment session.

In some embodiments, the ultrasound energy is applied at a mechanical index ranging from 0.05 to 2.3. In some embodiments, the ultrasound energy is applied at a mechanical index ranging from 0.05 to 1.8. In some embodiments, the ultrasound energy is applied at any suitable mechanical index. In some embodiments, the ultrasound energy is applied at any suitable mechanical index for disrupting the microbubbles and facilitating entry of the payload into the target cell(s). In some embodiments, the ultrasound energy is applied at lower mechanical index, and a higher mechanical index, continuously, in alternating pulses. In some embodiments, the lower mechanical index ranges from 0.05 to about 0.4. In some embodiments, the higher mechanical index ranges from 1.6 to about 2.3. In some embodiments, the alternating pulses occur in intervals ranging from 0.9 μs (microseconds) to 10 s. In some embodiments, an ultrasound flash may have a mechanical index which is increased relative to a first mechanical index. In some embodiments, administering ultrasound energy comprises administering at least four ultrasound flashes at a mechanical index that is increased relative to a first mechanical index with an interval between flashes of less than 10 seconds. In some embodiments, administering ultrasound energy comprises administering ultrasound flashes with a duration of about 0.9 us to about 2.5 us. On some embodiments, administering ultrasound energy comprises administering 10 pulses over 0.5 seconds with each pulse duration of about 2.2 us. In some embodiments, administering ultrasound energy comprises administering ultrasound at a first MI of about 0.07, and administering ultrasound flashes at a second MI of about 0.8 at a 10 frame duration, at a 4 second interval for a total of 9 flashes. In some embodiments, administering ultrasound energy comprises administering ultrasound at the second MI in at least 9 flashes. In some embodiments, administering ultrasound energy comprises administering ultrasound at the second MI in at least 18 flashes. In some embodiments, administering ultrasound energy comprises administering ultrasound at the second MI in at least 27 flashes.

In some embodiments, the ultrasound energy is applied for up to 30, 60, 90, 120, 150, 180, 240, 300, or 360 1200 seconds. In some embodiments, the ultrasound energy is applied for up to 30 seconds. In some embodiments, the ultrasound energy is applied for up to 60 seconds. In some embodiments, the ultrasound energy is applied for up to 90 seconds. In some embodiments, the ultrasound energy is applied for up to 120 seconds. In some embodiments, the ultrasound energy is applied for up to 150 seconds. In some embodiments, the ultrasound energy is applied for up to 180 seconds. In some embodiments, the ultrasound energy is applied for up to 240 seconds. In some embodiments, the ultrasound energy is applied for up to 300 seconds. In some embodiments, the ultrasound energy is applied for up to 360 seconds.

In some embodiments, the treatment session as determined by the length of continuous application of ultrasound energy is up to 30, 60, 90, 120, 150, 180, 240, 300, or 360 seconds. In some embodiments, the treatment session as determined by the length of continuous application of ultrasound energy is up to 30 seconds. In some embodiments, the treatment session as determined by the length of continuous application of ultrasound energy is up to 60 seconds. In some embodiments, the treatment session as determined by the length of continuous application of ultrasound energy is up to 90 seconds. In some embodiments, the treatment session as determined by the length of continuous application of ultrasound energy is up to 120 seconds. In some embodiments, the treatment session as determined by the length of continuous application of ultrasound energy is up to 150 seconds. In some embodiments, the treatment session as determined by the length of continuous application of ultrasound energy is up to 180 seconds. In some embodiments, the treatment session as determined by the length of continuous application of ultrasound energy is up to 240 seconds. In some embodiments, the treatment session as determined by the length of continuous application of ultrasound energy is up to 300 seconds. In some embodiments, the treatment session as determined by the length of continuous application of ultrasound energy is up to 360 seconds.

In some embodiments, the treatment session as determined by the length of continuous application of ultrasound energy is up to 5, 10, 15, 20, 25, 30, 35, or 40 minutes. In some embodiments, the treatment session as determined by the length of continuous application of ultrasound energy is up to 5 min. In some embodiments, the treatment session as determined by the length of continuous application of ultrasound energy is up to 10 min. In some embodiments, the treatment session as determined by the length of continuous application of ultrasound energy is up to 15 min. In some embodiments, the treatment session as determined by the length of continuous application of ultrasound energy is up to 20 min. In some embodiments, the treatment session as determined by the length of continuous application of ultrasound energy is up to 25 min. In some embodiments, the treatment session as determined by the length of continuous application of ultrasound energy is up to 30 min. In some embodiments, the treatment session as determined by the length of continuous application of ultrasound energy is up to 35 min. In some embodiments, the treatment session as determined by the length of continuous application of ultrasound energy is up to 40 min.

As stated herein, undesirable effects on living cells or tissues can occur due to ultrasound applications, including repeated ultrasound applications in repeated sonoporation treatment sessions. In some embodiments, the present disclosure provides methods for improvement of gene transfection that does not result in substantial DNA or cell damage in the target cells, tissues, or organs. In some embodiments, the method does not result in substantial cellular damage to the target cell. In some embodiments, the method does not result in inflammation of the target tissue, or in the elevation of apoptotic or inflammatory biomarkers. Cellular damage can be detected using apoptotic biomarkers. For example, in the liver, detection of released hepatocellular transaminases, e.g., serum alanine aminotransferase (ALT) or aspartate aminotransferase (AST), can be an indicator of apoptotic hepatocytes. Additional apoptotic biomarkers may comprise interleukin 6 (IL6) or B-cell lymphoma 2 (BCL2 or BCL2 apoptosis regulator). In some embodiments, biomarkers of cellular damage, inflammation, or death are not detected at apoptotic levels during or in a time period following application of the sonoporation treatment methods disclosed herein. In some embodiments, biomarkers of cell damage are not detected at clinically elevated levels during or in a time period immediately following application of the method disclosed herein. In some embodiments, repeating the treatment session does not result in substantial cell damage, cellular inflammation, or cell death. In some embodiments, repeating the treatment session does not result in a substantial elevation of inflammatory biomarker(s). In some embodiments, repeating a sonoporation treatment results in elevation of inflammatory biomarkers within 20% of a baseline level of an inflammatory biomarker(s). Non-limiting examples of markers of cellular damage, inflammation, or apoptosis include ALT, AST, IL6, and BCL2. In some embodiments, repeating the treatment session does not result in a substantial, clinically significant, other elevation of ALT, AST, IL6, or BCL2. In some embodiments, the following biomarkers for cellular damage are not detected at apoptotic levels following a treatment session (e.g., administering the nucleic acid payload to the subject; administering a plurality of microbubbles to the subject; and administering ultrasound energy to the subject in proximity to the target cell(s), and, optionally, administering ultrasound energy to the subject in proximity to target cell(s) at a second location in a target tissue): ALT, AST, IL6, BCL2, or combinations thereof, and, optionally wherein the target cell is in a liver. In some embodiments, the following biomarkers for cellular damage are not clinically elevated following a treatment session: ALT, AST, IL6, BCL2, or combinations thereof, and, optionally wherein the target cell is in a liver. In some embodiments, the following biomarkers for cellular damage are not clinically elevated following a treatment session: creatinine levels in urine, albumin to creatine ratio in urine, creatinine levels in blood, a glomerular filtration rate, blood in urine, protein levels in urine, or an osmolality of urine, and, optionally wherein the target cell is in a kidney. In some embodiments, the following biomarkers for cellular damage are not clinically elevated following a treatment session: troponin levels in blood, or creatinine phosphokinase, and, optionally wherein the target cell is in a heart or skeletal muscle.

In some embodiments, the method comprises administering ultrasound energy transcutaneously to the subject in proximity to one or more target cells. In some embodiments, the one or more target cells are hepatic cells. In some embodiments, the one or more target cells are renal cells. In some embodiments, the one or more target cells are pancreatic cells. In some embodiments, the one or more target cells are cardiac cells. In some embodiments, the one or more target cells are myocytes. In some embodiments, the one or more target cells are neuronal cells. In some embodiments, the one or more target cells are brain cells. In some embodiments, the one or more target cells are blood cells (e.g., white blood cells). In some embodiments, the target cells are cancerous cells.

In some embodiments, the one or more target cells(s) are comprised in a tissue. In some embodiments, the tissue is skeletal muscle tissue. In some embodiments, the tissue is smooth muscle tissue. In some embodiments, the tissue is connective tissue. In some embodiments, the tissue is lymphatic tissue. In some embodiments, the tissue is nervous tissue. In some embodiments, the tissue is diseased tissue, e.g., cancerous tissue, fibrotic tissue, or tissue otherwise in need of gene therapy.

In some embodiments, the target tissue is comprised in an organ. In some embodiments, the organ is the liver. In some embodiments, the organ is a kidney. In some embodiments, the organ is the pancreas. In some embodiments, the organ is the heart. In some embodiments, the organ is the brain.

In some embodiments, the one or more target cells are comprised in a tumor. In some embodiments, the tumor is a solid tumor. In some embodiments, the tumor is a liquid tumor.

In some embodiments, after administering of the nucleic acid construct and sonoactive microstructures, the ultrasound acoustic energy is applied at the target cell, tissue, or organ. In some embodiments, the ultrasound acoustic energy is applied at the target cell, tissue, or organ during administration of the nucleic acid construct, the sonoactive microstructures or both. In some embodiments, the ultrasound acoustic energy is applied at the target cell, tissue, or organ during and after administration of the nucleic acid construct, the sonoactive microstructures or both.

In some embodiments, the method comprises administering an effective amount of ultrasound energy transcutaneously in proximity to target cell(s) at a first and a second location in a target tissue. In some embodiments, the first and the second location are different regions of the target tissue (e.g., a dorsal and a ventral region of the target tissue, or a first and a second lobe of the target tissue). In some embodiments, the first and the second location are in the same or similar region of the target tissue (e.g., the first and the second location are at a dorsal region of the target tissue, or the first and the second locations are in the same lobe of the target tissue).

In some embodiments, the first location in the target tissue and the third location in the target tissue are a same location. In some embodiments, the first location in the target tissue and the third location in the target tissue are different. In some embodiments, the first location in the target tissue and the subsequent location in the target tissue are a same location, in the first treatment session. In some embodiments, the first location in the target tissue and the subsequent location in the target tissue are the different, in the first treatment session. In some embodiments, the second location in the target tissue and the fourth location in the target tissue are a same location. In some embodiments, the second location in the target tissue and the fourth location in the target tissue are different. In some embodiments, in the second treatment session, in the target tissue and the subsequent location in the target tissue are a same location, in the second treatment session. In some embodiments, in the second treatment session, the third location in the target tissue and the subsequent location in the target tissue are different. In some embodiments, the fifth location is a same location as any one of the first to fourth locations. In some embodiments, the sixth is a same location as any one of the first to fourth locations. In some cases, the first location and the second location are contiguous locations of the target tissue. In some cases, the second location and the subsequent location are contiguous locations of the target tissue. In some cases, the third location and the fourth location are contiguous locations of the target tissue. In some cases, the fourth location and the subsequent location are contiguous locations of the target tissue. In some cases, the fifth location and sixth location are contiguous locations of the target tissue. In some cases, the sixth location and the subsequent location are contiguous locations of the target tissue. In some cases, the fifth location or the sixth location are contiguous with any one of the first location to the fourth location. In some embodiments, administering ultrasound energy to the subject at the first location and at the second location comprises moving an ultrasound probe across a surface of the subject's skin from the first location to the second location.

In some embodiments, the target tissue is the liver and a first location is one of the right lobe, the left lobe, the caudal lobe, the caudate lobe, or the medial lobe, and the second location is in a different lobe than the first location. In some embodiments, a first location is one of the right lobe, the left lobe, the caudal lobe, or the medial lobe, and the second location is in a different area of the same lobe as the first location (e.g., the first and second locations are different areas of the right lobe). In some embodiments, the first location and the second location are in the same area of the same lobe. In some embodiments, the target cell(s) and/or the target tissue are in a liver. In some embodiments, the first location is a first lobe of the liver and the second location or subsequent location is a second lobe or a subsequent lobe of the liver. In some embodiments, the first lobe of the liver is a right lobe, wherein the second lobe is a left lobe, and wherein the subsequent lobe is one or both of a caudate lobe or a quadrate lobe. In some embodiments, the nucleic acid payload comprises a therapeutic transgene, wherein target cell is a hepatocyte, and wherein at least 50% of cells expressing the therapeutic transgene in the liver are hepatocytes.

In some embodiments, the target tissue is the kidney and the first location is a lateral face of the kidney, and the second location is a different region than the first location. In some embodiments, the second location is a central region of the kidney. In some embodiments, a second location or subsequent location is a longitudinal face of the kidney. In some embodiments, the second location or subsequent location is a face at least 45 degrees about an axis of rotation about they kidney. In some embodiments, the second location or subsequent location is a face about 90 degrees about an axis of rotation about the kidney.

In some embodiments, the target cell(s) and/or the target tissue are in a kidney. In some embodiments, the first location is in a first region of the kidney and the second location is in a second region of the kidney. In some embodiments, expression of the nucleic acid payload is induced in multiple cell types in the kidney. In some embodiments, a transgene encoded by the nucleic acid payload is expressed in cells in all regions of the kidney. In some embodiments, a transgene encoded by the nucleic acid payload is expressed in cells across four non-overlapping spatial regions of the kidney, the four non-overlapping spatial regions of the kidney defining an entire kidney.

In some embodiments, the target tissue exhibits a cystic pathology. In some embodiments, delivery and/or expression of the nucleic acid payload to the target cell(s) in the target tissue exhibiting the cystic pathology is increased.

In some embodiments, the target tissue is the pancreas and a first location is one of the head, neck, body, or tail region, and the second location is in a different region than the first location. In some embodiments, a first location is one of the head, neck, body, or tail region, and the second location is in a different area of the same region as the first location (e.g., the first and second locations are different areas of head region). In some embodiments, the first location and the second location are in the same area of the same region.

In some embodiments, a distance between the first location in the target tissue and the second location or subsequent location in the target tissue is at least 25% of a maximum distance of a major axis of an organ comprising the target tissue. In some embodiments, a distance between the third location in the target tissue and the fourth location or subsequent location in the target tissue is at least 25% of a maximum distance of major axis of an organ comprising the target tissue. In some embodiments, a distance between the first location in the target tissue and the second location or subsequent location in the target tissue is at least 1, 2, or 3 cm. In some embodiments, a distance between the third location in the target tissue and the fourth location or subsequent location in the target tissue is at least 1, 2, or 3 cm. In some embodiments, administering the ultrasound energy at the second location or subsequent location in the target tissue increases microvascular perfusion of the target tissue. As used herein a “major axis of an organ” is a maximum length of the longest line that can be drawn across a target organ from one end of the organ to an opposite end.

In some embodiments, a distance between the first location in the target tissue and the second location or subsequent location in the target tissue is at least the diameter of the ultrasound focal beam.

In some embodiments, the method further comprises the method further comprises administering ultrasound energy transcutaneously to the subject in proximity to target cell(s) at a third location in the target tissue. In some embodiments, the method further comprises administering intravenously through a peripheral vein a third dose of i) the payload, and ii) the plurality of sonoactive microstructures to the subject.

In some embodiments, administration of the third dose and/or administration of an effective amount of ultrasound energy at the third location increases expression of a nucleic acid or amino acid sequence encoded by the payload by at least 5%, at least 10%, at least 20%, at least 30%, at least 40%, at least 50%, or more as compared to administration of a single dose or two doses. In some embodiments, administration of the third dose and/or administration of an effective amount of ultrasound energy at the third location increases expression of the nucleic acid or amino acid sequence encoded by the payload by at least 5%, at least 10%, at least 20%, at least 30%, at least 40%, at least 50%, or more within 24 hours as compared to administration of a single dose or two doses. In some embodiments, administration of the third dose and/or administration of an effective amount of ultrasound energy at the third location increases expression of the nucleic acid or amino acid sequence encoded by the payload by at least 5%, at least 10%, at least 20%, at least 30%, at least 40%, at least 50%, or more for 48 hours as compared to administration of a single dose or two doses. In some embodiments, administration of the third dose and/or administration of an effective amount of ultrasound energy at the third location increases expression of the nucleic acid or amino acid sequence encoded by the payload by at least 5%, at least 10%, at least 20%, at least 30%, at least 40%, at least 50%, or more for 5 days as compared to administration of a single dose or two doses. In some embodiments, administration of the third dose and/or administration of an effective amount of ultrasound energy at the third location increases expression of the nucleic acid or amino acid sequence encoded by the payload by at least 5%, at least 10%, at least 20%, at least 30%, at least 40%, at least 50%, or more for 7 days as compared to administration of a single dose or two doses.

In some cases, administering the second treatment session increases delivery or expression of the nucleic acid payload by at least 25%, 50%, 75%, 100%, 250%, 300%, 350%, 400%, 450%, or 500%, as compared to a method comprising administration of the first treatment session without the second treatment session. In some cases, administering the subsequent treatment session increases delivery or expression of the nucleic acid payload by at least 100%, 250%, 300%, 350%, 400%, 450%, or 500%, as compared to a method comprising administration of the first treatment session without the second treatment session, or as compared to a method comprising administration of the first treatment session and the second treatment session without administration of the subsequent treatment session.

In some embodiments, the first therapeutic composition and the second therapeutic composition are a same therapeutic composition. In some embodiments, the first therapeutic composition and the second therapeutic composition comprise different dosages of the nucleic acid payload. In some embodiments, the first therapeutic composition and the second therapeutic composition comprise different dosages of sonoactive agents, or different sonoactive agents. In some embodiments, administering ultrasound energy to the subject at the first location and at the second location comprises moving an ultrasound probe across a surface of the subject's skin from the first location to the second location.

In some embodiments, the nucleic acid payload comprises a nonendogenous gene. In some embodiments, the transgene comprises a detectible marker. In some embodiments, the transgene comprises luciferase. In some embodiments, inducing expression of the nucleic acid payload comprises inducing expression of luciferase. In some embodiments, the transgene comprises green fluorescent protein. In some embodiments, inducing expression of the nucleic acid payload comprises inducing expression of green fluorescent protein.

In some embodiments, the nucleic acid payload is configured to perform gene augmentation, gene replacement, gene editing, base editing, base knockdown, gene knock down, or gene knockout. In some embodiments, delivering the nucleic acid payload to the target cell of the subject increases or decreases expression of a gene in the target cell. In some embodiments, the nucleic acid payload can be a therapeutic payload. In some embodiments, the nucleic acid construct is a miniplasmid comprising a nucleic acid payload. In some embodiments, the nucleic acid payload comprises a transgene. In some embodiments, the transgene may be an endogenous transgene. In some embodiments, the transgene may be a non-endogenous transgene. In some embodiments, the transgene is a therapeutic transgene.

In some embodiments, the transgene comprises a therapeutic RNA. In some embodiments, the therapeutic RNA is an mRNA. In some embodiments, the therapeutic RNA is an RNA interference (RNAi) agent, e.g., a double-stranded RNA, a single-stranded RNA, a micro RNA (miRNA), a short interfering RNA (siRNA), short hairpin RNA (shRNA), or a triplex-forming oligonucleotide. In some embodiments, the therapeutic RNA is a catalytically active RNA molecule (ribozyme). In some embodiments, the therapeutic RNA is a transfer RNA (tRNA). In some embodiments, the therapeutic RNA comprises one or more chemical modifications (e.g., one or more modified nucleobases, nucleosides, or nucleotides).

In some embodiments, the transgene comprises one or more components of a gene editing system. In some embodiments, the payload comprises a nuclease or engineered nuclease suitable for gene editing. In some embodiments, the nuclease is delivered as a polypeptide. In some embodiments, the nuclease is delivered as a nucleic acid encoding the nuclease. In some embodiments, the gene editing system is a CRISPR/Cas system. In some embodiments, the payload comprises a gRNA or a nucleic acid molecule encoding a gRNA (e.g., a plasmid encoding the gRNA). In some embodiments, the payload comprises a Cas protein or homologs or variants thereof, or a nucleic acid molecule encoding the Cas protein or homologs or variants thereof. In some embodiments, the payload comprises a TALEN or a nucleic acid molecule encoding the TALEN. In some embodiments, the payload comprises a zinc-finger nuclease (ZFN) or a nucleic acid encoding the ZFN. In some embodiments, the nuclease is an engineered nuclease. In some embodiments, the engineered nuclease is catalytically inactive. In some embodiments, the engineered nuclease is a fusion protein comprising the engineered nuclease a regulatory protein or an enzyme, or a functional domain thereof (e.g., a nuclease fused to a transcriptional regulatory domain or a nuclease fused to a deaminase) In some embodiments, the payload may further comprise a template DNA molecule suitable for knock-in to the subject's genome via non-homologous end joining (NHEJ) or homology directed repair (HDR).

In some embodiments, the payload comprises a nucleic acid that exceeds the size limitation of conventional gene therapy vectors. In some embodiments, the payload exceeds the size limitation of an adeno-associated viral vector (AAV). In some embodiments, the payload is greater than about 4.7 kb. In some embodiments, the payload is greater than about 5, about 5.5, about 6, about 6.5, about 7, about 7.5, about 8, about 8.5, about 9, about 9.5, about 10, about 10.5, about 11, about 11.5, about 12, about 12.5, or about 13 kb.

In some embodiments, the transgene comprises a therapeutic transgene. In some embodiments, the therapeutic transgene comprises: GLP-1, INS, Reg3g, MafA, PDX-1, NUEROG3, NGN3, DRYK, DYRKIA, DYRKIB, Factor VIII (FVIII), Factor IX (FIX), or combinations thereof. In some embodiments, the therapeutic transgene comprises GLP-1. In some embodiments, the therapeutic transgene comprises INS. In some embodiments, the therapeutic transgene comprises REg3g. In some embodiments, the therapeutic transgene comprises MafA. In some embodiments, the therapeutic transgene comprises PDX-1. In some embodiments, the therapeutic transgene comprises NUEROG3. In some embodiments, the therapeutic transgene comprises NGN3. In some embodiments, the therapeutic transgene comprises DYRKIA. In some embodiments, the therapeutic transgene comprises DYRKIB. In some embodiments, the therapeutic transgene comprises Factor VIII. In some embodiments, the therapeutic transgene comprises Factor IX.

In some embodiments, the therapeutic transgene comprises: PKD1, PKD2, COL4A3, COL4A4, COL4A5, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS or combinations thereof. In some embodiments, the therapeutic transgene comprises PKD1. In some embodiments, the therapeutic transgene comprises PKD2. In some embodiments, the therapeutic transgene comprises COL4A3. In some embodiments, the therapeutic transgene comprises COL4A4. In some embodiments, the therapeutic transgene comprises COL4A5. In some embodiments, the therapeutic transgene comprises Klotho. In some embodiments, the therapeutic transgene comprises Smad7. In some embodiments, the therapeutic transgene comprises TGF-beta. In some embodiments, the therapeutic transgene comprises SLC7A1. In some embodiments, the therapeutic transgene comprises SLC7A9. In some embodiments, the therapeutic transgene comprises SLC12A1. In some embodiments, the therapeutic transgene comprises UMOD. In some embodiments, the therapeutic transgene comprises REN. In some embodiments, the therapeutic transgene comprises HNF1B. In some embodiments, the therapeutic transgene comprises MUC1. In some embodiments, the therapeutic transgene comprises KCNJ1. In some embodiments, the therapeutic transgene comprises CLCNKA. In some embodiments, the therapeutic transgene comprises CLCNKB. In some embodiments, the therapeutic transgene comprises BSND. In some embodiments, the therapeutic transgene comprises NPHS1. In some embodiments, the therapeutic transgene comprises NPHS2. In some embodiments, the therapeutic transgene comprises CNTS.

In some embodiments, the payload comprises a therapeutic transgene coupled to one or more gene regulatory elements. In some embodiments, the gene regulatory element may be a promoter, enhancer, ribosome binding site, or transcription termination signal. In some embodiments, the nucleic acid payload comprises a therapeutic transgene coupled to a promoter. In some embodiments, a nucleic acid payload comprises a regulatory element such as a promoter, (e.g., APOE-ATT). In some embodiments, the nucleic acid payload comprises a therapeutic transgene coupled to a promoter other than CMV promoter or ubiquitin C (UbC) promoter. In some embodiments, the nucleic acid construct comprises a regulatory element such as a promoter, enhancer, ribosome binding site, or transcription termination signal. Examples of promoters contemplated herein include, but are not limited to, e.g., CMV promoter, UbC promoter, CAG promoter, EF-1a promoter, ApoE promoter, AAT-promoterApoE-AAT1 promoter, 3XSERP promoter, P3-hybrid promoter, or combinations thereof. In some embodiments, the nucleic acid construct comprises a promoter sequence comprising CAG. In some embodiments, the nucleic acid construct comprises a promoter sequence comprising ApoE. In some embodiments, the nucleic acid construct comprises a promoter sequence comprising SERP. In some embodiments, the nucleic acid construct comprises a promoter sequence comprising P3.

In some embodiments, the nucleic acid payload and the plurality of microbubbles are administered in a volumetric ratio of at least 1 part nucleic acid payload solution to 4 parts microbubble solution. In some embodiments, the nucleic acid payload and the plurality of microbubbles are administered in a volumetric ratio of about 1 part nucleic acid payload solution to 3 parts microbubble solution. In some embodiments, the nucleic acid payload and the plurality of microbubbles are administered in a volumetric ratio of about 1 part nucleic acid payload solution to 2 parts microbubble solution. In some embodiments, the nucleic acid payload and the plurality of microbubbles are administered in a volumetric ratio of about 1 part nucleic acid payload solution to 1 part microbubble solution.

In some embodiments, the administering of the nucleic acid construct and the sonoactive microstructures is by intravenous administration. In some embodiments, the intravenous administration is through a peripheral vein. In some embodiments, a peripheral vein is any vein outside of the torso. Exemplary peripheral veins include, but are not limited to, the jugular vein, the brachiocephalic veins, the saphenous vein, and veins of the forearm, head, and feet. Benefits of peripheral vein administration results in lower hydrostatic pressure and levels of peak negative pressure within the target tissue/organ as compared to other methods of administration, for example portal vein or inferior vena cava injections. Peripheral vein injections can promote serial injections within using invasive access and can promote the methods of repeating sonoporation disclosed herein. However, administration through a peripheral vein may not result in delivery, or in substantially reduced delivery, of the microbubbles or nucleic acid payload to the target tissue or cells. When administering the nucleic acid payload and microbubbles intravenously through a peripheral vein using the methods disclosed herein, sonoporation treatment sessions can be repeated without resulting in substantial cellular damage, inflammation, and/or death following the treatment session. When administering the nucleic acid payload and microbubbles intravenously through a peripheral vein in a treatment session using the methods disclosed herein, transfection of nucleic acid payload to a cell can be increased, and expression of the nucleic acid or amino acid sequence encoded by the nucleic acid payload may be increased.

In some embodiments, inducing expression of the nucleic acid payload comprises inducing production of a nucleotide sequence encoded by the payload. In some embodiments, inducing expression of the nucleic acid payload comprises inducing production of RNA encoded by the payload. In some embodiments, inducing expression of the nucleic acid payload comprises inducing production of messenger RNA (mRNA) encoded by the payload. In some embodiments, inducing expression of the nucleic acid payload comprises inducing production of small interfering RNA (siRNA) encoded by the payload. In some embodiments, inducing expression of the nucleic acid payload comprises inducing production of small hairpin RNA (shRNA) encoded by the payload.

In some embodiments, inducing expression of the nucleic acid payload comprises inducing production of protein encoded by the payload. In some embodiments, regulating expression of the nucleic acid payload comprises regulating production of RNA encoded by the payload. In some embodiments, regulating expression of the nucleic acid payload comprises regulating production of protein encoded by the nucleic acid payload, or regulating production of protein encoded by an mRNA encoded by the nucleic acid payload.

The methods described herein can be used to treat a subject in need for gene therapy or enzyme replacement treatment. In some aspects, the present disclosure provides methods of treating a subject having a liver condition. In some embodiments, the liver condition treated is: Wilson's Disease, Cholestasis progressive familial intrahepatic, Von Willebrand disease, Hemophilia A, Hemophilia B, Factor 5 deficiency, Alpha-Mannosidosis, Gaucher disease (glucocerebrosidase deficiency, glucocerebrosidosis), Niemann Pick Disease A/B, Carbamoylphosphate Synthetase I Deficiency, Glycogen Storage Disease Type III, Cystinosis, A1AT deficiency, Citrullinemia Type I & II.

A sonoporation treatment using the methods described herein can be used to treat a subject in need for gene therapy or enzyme replacement treatment. In another aspect, the present disclosure provides methods of treating a subject having a liver condition. In some embodiments, the liver condition treated is: Wilson's Disease, Cholestasis progressive familial intrahepatic, Von Willebrand disease, Hemophilia A, Hemophilia B, Factor 5 deficiency, Alpha-Mannosidosis, Gaucher's (glucocerebrosidase deficiency, glucocerebrosidosis), Niemann Pick Disease A/B, Carbamoylphosphate Synthetase I Deficiency, Glycogen Storage Disease Type III, Cystinosis, A1AT deficiency, Citrullinemia Type I & II.

In some embodiments, the present disclosure provides methods of treating a subject having a liver condition with a therapeutic transgene. In some embodiments, the therapeutic transgene encodes one or more of: ATP7B; ABCB11; ABCB4; ATP8B1; TJP2; VWF; FVIII; FIX; F5; MAN2B1; GBA; SMPD1; CPS1; GDE/AGL; CTNS; SERPINA1; ASS1, and/or SLC25A13.

In some embodiments, the present disclosure provides methods of treating a subject having a liver condition with a therapeutic transgene. In some embodiments, the liver condition is Wilson's Disease, and the therapeutic transgene encodes ATP7B. In some embodiments, the liver condition is Cholestasis, progressive familial intrahepatic (PFIC1-4) and the therapeutic transgene encodes one or more of ABCB11, ABCB4, ATP8B1 and/or TJP2. In some embodiments, the liver condition is Von Willebrand Disease, and the therapeutic transgene encodes VWF. In some embodiments, the liver condition is Hemophilia A, and the therapeutic transgene encodes FVIII. In some embodiments, the liver condition is Hemophilia B, and the therapeutic transgene encodes FIX. In some embodiments, the liver condition is Factor V Deficiency, and the therapeutic transgene encodes F5. In some embodiments, the liver condition is Alpha-Mannosidosis, and the therapeutic transgene encodes MAN2B1. In some embodiments, the liver condition is Gaucher's (glucocerebrosidase deficiency, glucocerebrosidosis), and the therapeutic transgene encodes GBA. In some embodiments, the liver condition is Niemann Pick Disease A/B, and the therapeutic transgene encodes SMPD1. In some embodiments, the liver condition is Carbamoylphosphate Synthetase I Deficiency, and the therapeutic transgene encodes CPS1. In some embodiments, the liver condition is Glycogen Storage Disease Type III, and the therapeutic transgene encodes GDE/AGL. In some embodiments, the liver condition is Cystinosis, and the therapeutic transgene encodes CTNS. In some embodiments, the liver condition is A1AT deficiency, and the therapeutic transgene encodes SERPINA1. In some embodiments, the liver condition is Citrullinemia Type I & II, and the therapeutic transgene encodes one or more of ASS1 and/or SLC25A13.

The methods described herein can be used to treat a subject in need for gene therapy or enzyme replacement treatment. In some aspects, the present disclosure provides methods of treating a subject having a liver condition. In some embodiments, the liver condition is treated by transfection of a therapeutic transgene, where therapeutic transgene (e.g., one or more of the transgenes) is: ATP7B (1465AA); ABCB11 (1321AA); ABCB4 (1286AA); ATP8B1 (1251AA); TJP2 (1190AA); VWF (2813AA); FVIII (2351AA); FIX (415AA); F5 (2224AA); MAN2B1 (1011AA); GBA (536AA); SMPD1 (631AA); CPS1 (1500AA); GDE/AGL (1532AA); CTNS (367AA); SERPINA1 (418AA); ASS1 (412AA), SLC25A13 (675AA).

In some embodiments, the present disclosure provides methods of treating a subject having a liver condition with payload comprising a therapeutic transgene. In some embodiments, the liver condition is Wilson's Disease, and the therapeutic transgene encodes ATP7B (1465 aa). In some embodiments, the liver condition is Cholestasis, progressive familial intrahepatic (PFIC1-4) and the therapeutic transgene encodes one or more of ABCB11 (1321 aa), ABCB4 (1286 aa), ATP8B1 (1251 aa) and/or TJP2 (1190 aa). In some embodiments, the liver condition is Von Willebrand Disease, and the therapeutic transgene encodes VWF (2813 aa). In some embodiments, the liver condition is Hemophilia A, and the therapeutic transgene encodes FVIII (2351 aa). In some embodiments, the liver condition is Hemophilia B, and the therapeutic transgene encodes FIX (415 aa). In some embodiments, the liver condition is Factor V Deficiency, and the therapeutic transgene encodes F5 (2224 aa). In some embodiments, the liver condition is Alpha-Mannosidosis, and the therapeutic transgene encodes MAN2B1 (1011 aa). In some embodiments, the liver condition is Gaucher's (glucocerebrosidase deficiency, glucocerebrosidosis), and the therapeutic transgene encodes GBA (536 aa). In some embodiments, the liver condition is Niemann Pick Disease A/B, and the therapeutic transgene encodes SMPD1 (631 aa). In some embodiments, the liver condition is Carbamoylphosphate Synthetase I Deficiency, and the therapeutic transgene encodes CPS1 (1500 aa). In some embodiments, the liver condition is Glycogen Storage Disease Type III, and the therapeutic transgene encodes GDE/AGL (1532 aa). In some embodiments, the liver condition is Cystinosis, and the therapeutic transgene encodes CTNS (367 aa). In some embodiments, the liver condition is A1AT deficiency, and the therapeutic transgene encodes SERPINA1 (418 aa). In some embodiments, the liver condition is Citrullinemia Type I & II, and the therapeutic transgene encodes one or more of ASS1 (412 aa) and/or SLC25A13 (675 aa). In some embodiments, provided herein is a method of treating a subject with a liver condition, comprising providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session at least once more than 6 hours after but within 10 days after initiation of a first treatment session.

In some embodiments, provided herein is a method of treating a subject having Hemophilia A comprising providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session at least once more than 6 hours after but within 10 days after initiation of a first treatment session. In some embodiments, the therapeutic transgene is operably linked to a liver specific promoter. In some embodiments, the therapeutic transgene comprises a nucleic acid sequence encoding Factor VIII. In some embodiments, the nucleic acid payload and/or the plurality of microbubbles are delivered systemically. In some embodiments, the nucleic acid payload and/or the plurality of microbubbles are delivered intravenously.

In some embodiments, provided herein is a method of treating a subject having Wilson's Disease comprising providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session at least once more than 6 hours after but within 10 days after initiation of a first treatment session. In some embodiments, the therapeutic transgene is operably linked to a liver specific promoter. In some embodiments, the therapeutic transgene comprises a nucleic acid sequence encoding ATP7B. In some embodiments, the nucleic acid payload and/or the plurality of microbubbles are delivered systemically. In some embodiments, the nucleic acid payload and/or the plurality of microbubbles are delivered intravenously.

In one aspect, using the sonoporation methods described herein, the present disclosure provides methods of treating a subject having a kidney condition. In some embodiments, the kidney condition treated is: acute kidney injury (AKI), Alport Syndrome, or Autosomal Dominant Polycystic Kidney Disease (ADPKD).

In one aspect, using the sonoporation methods described herein, the present disclosure provides methods of treating a subject having a kidney condition. In some embodiments, the payload comprises a therapeutic transgene encoding one or more of COL4A3 (1670 aa), COL4A4 (1690 aa), COL4A5 (1685 aa), PKD1 (4303 aa) and/or PKD2 (968 aa).

In some embodiments, the present disclosure provides methods of treating a subject having a kidney condition with a therapeutic transgene. In some embodiments, the kidney condition is Alport Syndrome, and the therapeutic transgene encodes one or more of COL4A3, COL4A4, and/or COL4A5. In some embodiments, the kidney condition is Autosomal Dominant Polycystic Kidney Disease, and the therapeutic transgene encodes one or more of PKD1 and/or PKD2. In some embodiments, provided herein is a method of treating a subject with a kidney condition, comprising providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session at least once more than 6 hours after but within 10 days after initiation of a first treatment session. In some embodiments, provided herein is a method of treating a subject having Alport Syndrome comprising providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session at least once more than 6 hours after but within 10 days after initiation of a first treatment session. In some embodiments, the therapeutic transgene is operably linked to a liver specific promoter. In some embodiments, the therapeutic transgene comprises a nucleic acid sequence encoding COL4A3. In some embodiments, the therapeutic transgene comprises a nucleic acid sequence encoding COL4A4. In some embodiments, the therapeutic transgene comprises a nucleic acid sequence encoding COL4A5. In some embodiments, the nucleic acid payload and/or the plurality of microbubbles are delivered systemically. In some embodiments, the nucleic acid payload and/or the plurality of microbubbles are delivered intravenously.

In some embodiments, provided herein is a method of treating a subject having Autosomal Dominant Polycystic Kidney Disease comprising providing a treatment session to the subject, wherein the treatment session comprises: administering the nucleic acid payload to the subject; administering a plurality of microbubbles to the subject; and administering ultrasound energy to the subject in proximity to the target cell(s); and repeating the treatment session at least once more than 6 hours after but within 10 days after initiation of a first treatment session. In some embodiments, the therapeutic transgene is operably linked to a liver specific promoter. In some embodiments, the therapeutic transgene comprises a nucleic acid sequence encoding PKD1. In some embodiments, the nucleic acid payload and/or the plurality of microbubbles are delivered systemically. In some embodiments, the nucleic acid payload and/or the plurality of microbubbles are delivered intravenously.

In some embodiments, administering the ultrasound energy proximal to the sonoactive microstructures increases microvascular perfusion of a target tissue. In some embodiments, administering the ultrasound energy at the second location in the target tissue increases microvascular perfusion of a target tissue. In some embodiments, repeating the sonoporation treatment increases microvascular perfusion of a target tissue. In some embodiments, the target cell is in a target tissue exhibiting cystic pathology. In some embodiments, the target cell is in a target tissue exhibiting cystic pathology, wherein delivery of the nucleic acid to the target cell in the tissue exhibiting cystic pathology is increased. In some embodiments, target cell is comprised by a subject having polycystic kidney disease, wherein the therapeutic transgene comprises a sequence encoding PKD1 or PDK2, wherein delivery of the therapeutic transgene to the target cell is increased, thereby treating polycystic kidney disease.

In some embodiments, the nucleic acid construct is a miniplasmid. As used herein, the term “miniplasmid (mpDNA)” refers to nucleic acid constructs that are smaller in size (i.e., contain fewer base pairs (bp)) than conventional plasmids or pDNA, with regard to the size of the nucleic acid construct excluding the therapeutic transgene, promoters, and regulatory elements. In some embodiments, mpDNA constructs comprise a backbone smaller than 1 kb. In some embodiments, mpDNA constructs are smaller than 1000 bp excluding an expression cassette. In some embodiments, mpDNA constructs comprise a backbone smaller than 500 bp. In some embodiments, mpDNA constructs are smaller than 500 bp excluding an expression cassette. In some embodiments, the miniplasmid does not comprise a bacterial origin of replication. As used herein, the term “Nanoplasmid™” (e.g., Nanoplasmid sourced from Aldevron, Fargo, South Dakota.) refers to a small mpDNA construct that comprises a plasmid backbone that is less than 500 bp and does not comprise an antibiotic resistance gene.

Miniplasmid DNA nucleic acid constructs can be utilized to deliver an expression cassette, a transgene or a nonendogenous gene to cells in target cell-types, tissues, or organs. In some embodiments, the mpDNA construct comprises less than 500 base pairs excluding an expression cassette. In some embodiments, the mpDNA comprises less than 1000 base pairs excluding an expression cassette. In some embodiments, the miniplasmid comprises less than 500 base pairs excluding an expression cassette. In some embodiments, the mpDNA does not comprise antibiotic resistant genes. In some embodiments, the mpDNA construct does not comprise nucleotide sequences that encode bacterial genes. In some embodiments, the mpDNA construct does not comprise a bacterial genome. In some embodiments, the mpDNA construct comprises a therapeutic transgene and/or a regulatory element. In some embodiments, the mpDNA construct is a nanoplasmid (e.g., a Nanoplasmid™ sourced from Aldevron, Fargo, South Dakota). In some embodiments, the miniplasmid construct enhances the expression of a nonendogenous gene or a therapeutic transgene when used in conjunction with the claimed methods and ultrasound acoustic profiles. In some embodiments, the nanoplasmid construct enhances the expression of a nonendogenous gene or a therapeutic transgene. In some embodiments, durability of expression of a protein encoded by the nucleic acid payload may be increased relative to expression of the same protein in a larger plasmid (e.g., a plasmid of greater than 2 kb in length, excluding the transgene). In some embodiments, durability of expression of a protein encoded by the nucleic acid payload may be increased relative to expression of the same protein in another nucleic acid construct.

In some embodiments, the nucleic acid construct is a miniplasmid, (e.g., a construct comprising a backbone of less than 1000 bp or less than 500 bp) coupled to a nucleic acid payload.

In some embodiments, the nucleic acid payload comprises a HALO DNA construct. In some embodiments, the nucleic acid payload comprises a double stranded, covalently closed, circular DNA construct. In some embodiments, the nucleic acid payload comprises a DNA construct comprising structural elements facilitating nuclear entry. In some embodiments, the nucleic acid payload comprises a DNA construct comprising structural elements facilitating durable gene expression following gene transfection.

In some embodiments, the nucleic acid payload is a small linear DNA construct. In some embodiments, the small linear DNA construct lacks a bacterially derived backbone. In some embodiments, the small linear DNA construct is a non-viral capsid free vector. In some embodiments, the small linear DNA construct is an open linear DNA molecule. In some embodiments, the small linear DNA construct is a partially closed linear DNA molecule (e.g., the linear DNA construct is covalently closed on a first end and not covalently closed on the second end). In some embodiments, the small linear DNA construct is a closed linear DNA molecule (e.g., covalently closed). In some embodiments, the small linear DNA construct comprises a double stranded region comprising a DNA sequence of interest (e.g., a transgene, nonendogenous gene, or other heterologous sequence), flanked on one or both ends by a hairpin, stem loop, or single stranded loop.

In some embodiments, the nucleic acid payload is a closed linear DNA (“clDNA”) molecule comprising a stem region comprising a double stranded DNA sequence of interest covalently closed at both ends by hairpin loops. In some embodiments, the clDNA molecule is a single stranded covalently closed DNA molecule that forms a “dumbbell” or “doggy-bone” shaped structure under conditions allowing nucleotide hybridization. Therefore, although the clDNA is formed by a single stranded DNA molecule, the formation of the “dumbbell” structure by the hybridization of two complementary sequences within the same molecule generates a structure consisting on a double-stranded middle segment flanked by two single-stranded loops.

In some embodiments, the clDNA molecule is a capsid-free, linear duplex DNA molecules formed from a continuous strand of DNA with covalently-closed ends. In some embodiments, the clDNA molecule comprises a DNA sequence of interest (e.g., a transgene, nonendogenous gene, or other heterologous sequence) flanked by a 5′ inverted terminal repeat (ITR) sequence and a 3′ ITR sequence. In some embodiments, 5′ TR and 3′ ITR can have the same symmetrical three-dimensional organization with respect to each other, (e.g., symmetrical or substantially symmetrical). In some embodiments, 5′ ITR and 3′ ITR can have different three-dimensional organization with respect to each other (e.g., asymmetrical ITRs). In some embodiments, the clDNA molecule further comprises a nucleotide sequence encoding an inhibitor of an immune response. In some embodiments, the inhibitor inhibits an innate immune response.

In some embodiments, the clDNA can be apportioned into at least three sections. The first section and third sections include some complementarity, such that under appropriate or physiological conditions, these sections are capable of base-pairing and forming a duplex or stem structure. The duplex is a result of self-complementary sequences within the polynucleotide. The second section intervenes between the first and third sections, and includes the sequence for a linear single stranded nucleic acid for delivery. The second section, since it is positioned between the two complementary sequences, will generally form a “loop” of nucleic acid, which starts and ends at the first and third sections which are duplexed. It will generally be represented as a single strand, since no regions of complementarity with other sequences within the polynucleotide will intentionally be included in order for the delivery vector to work as intended. However, single stranded nucleic acids are well known for assuming some secondary structure, and therefore the second section may be in any possible conformation, including one or more of hairpins, loops, and pseudoknots, including sections of linear nucleic acid.

In some embodiments, the nucleic acid payload is a partially closed linear DNA molecule. In some embodiments, the partially closed linear DNA molecule comprises a linear portion of a double stranded DNA molecule, wherein a first end of the molecule is closed (or covalently closed, e.g., by a hairpin, stem loop, or single stranded loop) and a second end of the molecule is open (e.g., having exposed DNA ends). In some embodiments, the double stranded region comprises a DNA sequence of interest (e.g., a transgene, nonendogenous gene, or other heterologous sequence). In some embodiments, the partially closed linear DNA product comprises one or more nuclease-resistant nucleotides in the open end. In some embodiments, the open end refers to at least 5, at least 10, at least 15, or at least 20 base pairs located closest to the open end of the DNA product. In some embodiments, the partially closed linear DNA product comprises at least 5 nuclease-resistant nucleotides in the open-end region. A nuclease-resistant nucleotide refers to any suitable nucleotide that provides or enhances resistance to nuclease digestion (e.g., an exonuclease or endonuclease). In some embodiments, the nuclease-resistant nucleotide is a modified nucleotide. In some embodiments, the modified nucleotide is a phosphorothioated nucleotide (e.g., 2′-deoxynucleotides-5′-(a-thio)-triphosphate). A “phosphorothioated nucleotide” refers to a nucleotide that has an altered phosphate backbone, wherein, the sugar moieties are linked by a phosphorothioate bond. In the phosphate backbone of an oligonucleotide sequence, the phosphorothioate bond contains a sulphur atom as a substitute for a non-bridging oxygen atom. This modification renders the internucleotide linkage resistant to nuclease degradation.

In some embodiments, the nucleic acid payload is a circular RNA (circRNA) molecule. A circRNA is 3-5′ covalently closed RNA ring. In some embodiments, the circRNA has improved resistance to exonuclease-mediated degradation compared to a comparable linear RNA. In some embodiments, the circRNA has improved stability as compared to a comparable linear RNA. In some embodiments, the circRNA comprises an internal ribosomal entry site (IRES)

In some embodiments, any one of the nucleic acid payloads described herein comprises one or more modified nucleotides. In some embodiments, any one of the nucleic acid payloads described herein comprises at least two modified nucleotides. In some embodiments, the one or more modified nucleotides improve transfection efficiency, expression efficiency, stability, bioavailability, functional persistence, resistance to degradation, overall functional performance, or combinations thereof. A “modified nucleotide” is any nucleotide (e.g., adenosine, guanosine, cytidine, and thymidine) that has been chemically modified—by modification of the base, the sugar or the phosphate group- or that incorporates a non-natural moiety in its structure. Thus, the modified nucleotide may be naturally or non-naturally occurring depending on the modification. In some embodiments, the modified nucleotide is resistant to nuclease activity (e.g., an endo or exonuclease).

In some embodiments, a total amount of DNA administered to a subject for purposes of sonoporation can range from about 1 microgram (ug) to about 200 mg. In some embodiments, a total amount of DNA administered to a subject is 20 mg to 100 mg. In some embodiments, a total amount of DNA administered to a subject is about 20 mg. In some embodiments, a total amount of DNA administered to a subject is about 50 mg.

In some embodiments, the nucleic acid construct is administered at a dosage of at least 0.4 mg/kg. In some embodiments, the nucleic acid construct is administered at a dosage of up to 500 mg/kg. In some embodiments, the nucleic acid construct is administered at a dosage from about 0.5 mg/kg body mass to about 500 mg/kg body mass. In some embodiments, the nucleic acid construct is administered at a dosage of 2 mg/kg-5.5 mg/kg. In some embodiments, the nucleic acid construct is administered at a dosage of about 3.5 μg/μl. In some embodiments, about 2×10\hat{ }13 to about 3×10\hat{ }13 copies of the nucleic acid construct are administered to the subject.

Sonoactive agents (also referred to as sonoactive microstructures, acoustic microspheres, or microbubbles) contemplated herein include, but are not limited to, those used as ultrasonic imaging contrast agents. In some embodiments, the sonoactive microstructures comprise a phospholipid stabilized microstructure. In some embodiments, the phospholipid stabilized microstructure comprises a high molecular wight gas core, or a perflutran core. Examples of sonoactive microstructures include, but are not limited to, OPTISON (GE Healthcare), Sonazoid (GE Healthcare), or DEFINITY and Definity RT (Lantheus Medical Imaging, Inc). In some embodiments, the sonoactive microstructures are LUMASON (Bracco) (sulfur hexafluoride lipid-type A microspheres). In some embodiments, the sonoactive microstructures are Sono Vue (sulfur hexafluoride microbubbles). In some embodiments, the sonoactive microstructures comprise a protein stabilized microstructure. In some embodiments, the sonoactive microstructures are Optison microbubbles.

In some embodiments, the sonoactive agents (e.g., sonoactive microstructures or microbubbles) comprise a phospholipid stabilized microstructure. In some embodiments, the sonoactive agents (e.g., sonoactive microstructures or microbubbles) comprise a phospholipid stabilized shell. In some embodiments, the sonoactive agents (e.g., sonoactive microstructures or microbubbles) comprise a lipid stabilized shell. In some embodiments, the sonoactive agents (e.g., sonoactive microstructures or microbubbles) comprise a protein stabilized shell. In some embodiments, the sonoactive agents (e.g., sonoactive microstructures or microbubbles) comprise an albumin stabilized shell. In some embodiments, the phospholipid stabilized microstructure comprises a high molecular weight gas core, e.g., a perflutren core. Examples of sonoactive agents (e.g., sonoactive microstructures or microbubbles) include, but are not limited to, OPTISON (GE Healthcare), Sonazoid (GE Healthcare), or Definity and Definity RT (Lantheus Medical Imaging, Inc). In some embodiments, the sonoactive agents (e.g., sonoactive microstructures or microbubbles) are Sonazoid microbubbles, Definity microbubbles, or Definity RT microbubbles. In some embodiments, the sonoactive agents (e.g., sonoactive microstructures or microbubbles) are Optison microbubbles. In some embodiments, the sonoactive agents (e.g., sonoactive microstructures or microbubbles) are Sono Vue (sulfur hexafluoride microbubbles). In some embodiments, the sonoactive agents (e.g., sonoactive microstructures or microbubbles) comprise a protein stabilized microstructure. In some embodiments, the sonoactive agents (e.g., sonoactive microstructures or microbubbles) are Optison microbubbles.

The sonoactive agents (e.g., sonoactive microstructures or microbubbles) can be administered prior to, after, or simultaneous (e.g., co-administered) with the administration of the nucleic acid construct (or nucleic acid payload). In some embodiments, the nucleic acid construct and the sonoactive agents (e.g., sonoactive microstructures or microbubbles) are coadministered. In some embodiments, the administering of the nucleic acid construct and the sonoactive agents (e.g., sonoactive microstructures or microbubbles) occurs serially, concurrently, sequentially, or continuously. In some embodiments, the administering of the nucleic acid construct and the sonoactive agents (e.g., sonoactive microstructures or microbubbles) occurs serially. In some embodiments, the administering of the nucleic acid construct and the sonoactive agents (e.g., sonoactive microstructures or microbubbles) occurs concurrently. In some embodiments, the administering of the nucleic acid construct and the sonoactive agents (e.g., sonoactive microstructures or microbubbles) occurs sequentially. In some embodiments, the administering of the nucleic acid construct and the sonoactive agents (e.g., sonoactive microstructures or microbubbles) occurs during the course of the ultrasound treatment continuously (e.g., is administered intravenously throughout).

As used herein, concentrations of microstructures/mL refers to the concentration of the sonoactive agents (e.g., sonoactive microstructures or microbubbles) in a pharmaceutical composition immediately prior to administration to the subject. In some embodiments, the sonoactive microstructures are administered at a concentration of about 5×10\hat{ }8 to about 1.2×10\hat{ }10 microstructures/ml, for example, 1×10\hat{ }9 microstructure/ml of Definity RT. In some embodiments, the sonoactive microstructures are administered at a dosage of about 1-50 ml, for example, 1 ml of a protein stabilized sonoactive microstructure (e.g., Optison). In some embodiments, the protein stabilized sonoactive microstructure (e.g., Optison) comprise a diameter of 3-4.5 micrometers. The sonoactive microstructures may be administered at a concentration of about 500M (million) to about 800M microstructures per ml. In some embodiments, the 1×10\hat{ }9 of phospholipid stabilized sonoactive microstructures (e.g., Sonazoid) are administered. In some embodiments, the phospholipid stabilized sonoactive microstructures (e.g., Sonazoid) comprise a diameter of 1-5 micrometers. In some embodiments, the sonoactive microstructures are administered at a dose of about 0.1 to about 0.8 mg microstructures/kg of body mass. In some embodiments, the sonoactive microstructures are administered at a dose of about 0.1 to about 1.0 ml/kg of body mass. In some embodiments, the sonoactive microstructures are administered at a concentration of about 10\hat{ }9 microstructures/ml. In some embodiments, the sonoactive microstructures are administered at a concentration of at least 5×10\hat{ }8 microstructures per mL. In some embodiments, the sonoactive microstructures are administered at a concentration of up to 1.2×10\hat{ }10 microstructures/mL. In some embodiments, the sonoactive microstructures are administered at a concentration of 5×10\hat{ }8 to 8×10\hat{ }8 microstructures/ml.

In some embodiments, the nucleic acid construct and the sonoactive agents (e.g., sonoactive microstructures or microbubbles) are mixed prior to being coadministered. In some instances, the sonoactive microstructures are mixed with the nucleic acid constructs along with additional buffers or agents such as saline or other biocompatible solutions with varying electrostatic charges, surface chemistries, and ligands before administering to the subject. For example, Optison sonoactive microstructures can be mixed with a nanoplasmid and saline and administered together.

The present disclosure provides ultrasound systems comprising computer systems that are programmed to implement methods of the disclosure. The ultrasound systems 200 may be operably connected to one or more ultrasound transducers 211 controlled by a computer system 201 one or more computer processers 204 which may comprise one or more computer readable medium/media 205 which comprise instructions configured to cause the ultrasound systems to perform the methods of the present disclosure. The ultrasound systems 200 and/or the computer processers 204 may be in communication with the cloud 207 or other remote server which enable the remote operation and control of the ultrasound systems 200 and performance of the methods disclosed herein. The computer system 201 can be an electronic device of a user or a computer system that is remotely located with respect to the electronic device. The electronic device can be a mobile electronic device. The computer system includes a central processing unit (CPU, also “processor” and “computer processor” herein), which can be a single core or multi core processor, or a plurality of processors for parallel processing. The computer system also includes memory or memory location 206 (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

Aspects disclosed herein provide a system (e.g., ultrasound systems) comprising: an ultrasound transducer configured to apply ultrasound acoustic energy to a subject at a plurality of mechanical indexes; a computer system comprising a computer processor and a computer-readable medium, wherein the computer system is configured to implement a method of applying ultrasonic acoustic energy to a target cell of the subject, the method comprising: applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0), wherein the subject has been administered a nucleic acid construct comprising the nucleic acid payload, wherein the nucleic acid construct is a miniplasmid, and a plurality of sonoactive microstructures. In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In some embodiments, the nucleic acid construct is a plasmid that is less than or equal to 500 base pairs in length excluding an expression cassette, or wherein the wherein the nucleic acid construct is a miniplasmid. In some embodiments, applying the ultrasonic acoustic energy of at the first MI and applying the ultrasonic acoustic energy of at the second MI are repeated at least twice. In some embodiments, applying the ultrasonic acoustic energy of at the first MI and applying the ultrasonic acoustic energy at the second MI are repeated from 4 to 18 times. In some embodiments, applying the ultrasonic acoustic energy of at the first MI and applying the ultrasonic acoustic energy at the second MI are repeated from 6 to 12 times. In some embodiments, applying the ultrasonic acoustic energy of at the first MI and applying the ultrasonic acoustic energy at the second MI are repeated from 8 to 10 times. In some embodiments, the second MI ranges from about 1.4 to about 2.0. In some embodiments, applying the ultrasound acoustic energy of at the second mechanical index induces formation of a pore in a membrane of the cell. In some embodiments, applying the ultrasound acoustic energy of at the first mechanical index induces formation of an intercellular gap or an interendothelial gap. In some embodiments, an ultrasound transducer sends ultrasound acoustic energy or receives reflected ultrasound acoustic energy at least 95% of a period of time in which an ultrasound transducer continuously is contacting the subject. In some embodiments, applying the ultrasonic acoustic energy of d. comprises applying the ultrasonic acoustic energy at the second MI using a pulse. In some embodiments, applying the ultrasonic acoustic energy at the second MI comprises applying the ultrasonic acoustic energy at the second MI using a pulse with a duration of about 1 us to about 500 us. In some embodiments, applying the ultrasonic acoustic energy at the second MI comprises applying the ultrasonic acoustic energy at the second MI using a pulse with a duration of up to 200 us. In some embodiments, applying the ultrasonic acoustic energy at the second MI comprises applying the ultrasonic acoustic energy at the second MI using a pulse with a duration of up to 500 us. In some embodiments, applying the ultrasonic acoustic energy at the second MI comprises applying the ultrasonic acoustic energy at the second MI using a pulse with a duration of about 1 us to about 200 us. In some embodiments, applying the ultrasonic acoustic energy at the second MI comprises applying the ultrasonic acoustic energy at the second MI using a pulse with a duration of about 2.3 us. In some embodiments, the method includes repeating the applying the ultrasonic acoustic energy at the first MI, and the applying the ultrasonic acoustic energy at the second MI. In some embodiments, the repeating comprises applying the ultrasonic acoustic energy at the first MI for an amount of time sufficient to permit reperfusion of the sonoactive microstructures in a tissue comprising the target cell. In some embodiments, the repeating comprises applying the ultrasonic acoustic energy at the first MI for 1-30 seconds before repeating the applying the ultrasound acoustic energy at the second MI. In some embodiments, the repeating comprises applying the ultrasonic acoustic energy at the first MI for 5-15 seconds before applying the ultrasound acoustic energy at the second MI. In some embodiments, the repeating comprises applying the ultrasonic acoustic energy at the first MI for 10 seconds before applying the ultrasound acoustic energy at the second MI.

The systems (e.g., ultrasound systems) disclosed herein may be controlled or operated by a computer comprising a computer-readable medium configured to implement a method of applying ultrasonic acoustic energy to a target cell of the subject, the method comprising: applying an ultrasonic acoustic energy to the target cell at a first mechanical index (MI) that is up to 0.4; and applying an ultrasonic acoustic energy to the target cell at a second MI that is greater than 0.4 and up to 2.0, wherein the subject has been administered (1) a nucleic acid construct comprising a nucleic acid payload, wherein the nucleic acid construct is a miniplasmid, and (2) a plurality of sonoactive microstructures. In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In some embodiments, the miniplasmid is less than or equal to 500 base pairs in length excluding an expression cassette. In some embodiments, applying the ultrasonic acoustic energy at the first MI and applying the ultrasonic acoustic energy at the second MI are repeated at least twice. In some embodiments, applying the ultrasonic acoustic energy at the first MI and applying the ultrasonic acoustic energy at the second MI are repeated from 4 to 18 times. In some embodiments, applying the ultrasonic acoustic energy at the first MI and applying the ultrasonic acoustic energy at the second MI are repeated from 6 to 12 times. In some embodiments, applying the ultrasonic acoustic energy at the first MI and applying the ultrasonic acoustic energy at the second MI are repeated from 8 to 10 times. In some embodiments, the second MI ranges from about 1.4 to about 2.0. In some embodiments, applying the ultrasound acoustic energy at the second mechanical index induces formation of a pore in a membrane of the cell. In some embodiments, applying the ultrasound acoustic energy at the first mechanical index induces formation of an intercellular gap or an interendothelial gap. In some embodiments, an ultrasound transducer sends ultrasound acoustic energy or receives reflected ultrasound acoustic energy at least 95% of a period of time in which an ultrasound transducer continuously is contacting the subject. In some embodiments, applying the ultrasonic acoustic energy at the second MI comprises applying the ultrasonic acoustic energy at the second MI using a pulse. In some embodiments, applying the ultrasonic acoustic energy at the second MI comprises applying the ultrasonic acoustic energy at the second MI using a pulse with a duration of about 1 us to about 500 us. In some embodiments, applying the ultrasonic acoustic energy at the second MI comprises applying the ultrasonic acoustic energy at the second MI using a pulse with a duration of up to 200 us. In some embodiments, applying the ultrasonic acoustic energy at the second MI comprises applying the ultrasonic acoustic energy at the second MI using a pulse with a duration of up to 500 us. In some embodiments, applying the ultrasonic acoustic energy at the second MI comprises applying the ultrasonic acoustic energy at the second MI using a pulse with a duration of about 1 us to about 200 us. In some embodiments, applying the ultrasonic acoustic energy at the second MI comprises applying the ultrasonic acoustic energy at the second MI using a pulse with a duration of about 2.3 us. In some embodiments, the instructions comprise repeating the applying the ultrasonic acoustic energy at the first MI, and the applying the ultrasonic acoustic energy at the second MI. In some embodiments, the repeating comprises applying the ultrasonic acoustic energy at the first MI for an amount of time sufficient to permit reperfusion of the sonoactive microstructures in a tissue comprising the target cell. In some embodiments, wherein the repeating comprises applying the ultrasonic acoustic energy at the first MI for 1-30 seconds before repeating the applying the ultrasound acoustic energy at the second MI. In some embodiments, the repeating comprises applying the ultrasonic acoustic energy at the first MI for 5-15 seconds before applying the ultrasound acoustic energy at the second MI. In some embodiments, the repeating comprises applying the ultrasonic acoustic energy at the first MI for 10 seconds before applying the ultrasound acoustic energy at the second MI.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., hand-held device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, concentration of the analyte of interest. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit.

In some aspects, the disclosed provides quality control methods or methods to assess a risk associated with a food, with a hospital, with a clinic, or any other location where the presence of a bacterium poses a certain risk to one or more subjects. In many instances, systems, platforms, software, networks, and methods described herein include a digital processing device, or use of the same. In further embodiments, the digital processing device includes one or more hardware central processing units (CPUs), i.e., processors that carry out the device's functions, such as the automated sequencing apparatus disclosed herein or a computer system used in the analyses of a plurality of nucleic acid sequencing reads from samples derived from a food processing facility or from any other facility, such as a hospital a clinical or another. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In other embodiments, the digital processing device could be deployed on premise or remotely deployed in the cloud.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art. In many aspects, the disclosure contemplates any suitable digital processing device that can either be deployed to a food processing facility, or is used within said food processing facility to process and analyze a variety of nucleic acids from a variety of samples.

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, a digital processing device includes a digital camera. In some embodiments, a digital camera captures digital images. In some embodiments, the digital camera is an autofocus camera. In some embodiments, a digital camera is a charge-coupled device (CCD) camera. In further embodiments, a digital camera is a CCD video camera. In other embodiments, a digital camera is a complementary metal-oxide semiconductor (CMOS) camera. In some embodiments, a digital camera captures still images. In other embodiments, a digital camera captures video images. In various embodiments, suitable digital cameras include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and higher megapixel cameras, including increments therein. In some embodiments, a digital camera is a standard definition camera. In other embodiments, a digital camera is an HD video camera. In further embodiments, an HD video camera captures images with at least about 1280×about 720 pixels or at least about 1920×about 1080 pixels. In some embodiments, a digital camera captures color digital images. In other embodiments, a digital camera captures grayscale digital images. In various embodiments, digital images are stored in any suitable digital image format. Suitable digital image formats include, by way of non-limiting examples, Joint Photographic Experts Group (JPEG), JPEG 2000, Exchangeable image file format (Exif), Tagged Image File Format (TIFF), RAW, Portable Network Graphics (PNG), Graphics Interchange Format (GIF), Windows® bitmap (BMP), portable pixmap (PPM), portable graymap (PGM), portable bitmap file format (PBM), and WebP. In various embodiments, digital images are stored in any suitable digital video format. Suitable digital video formats include, by way of non-limiting examples, AVI, MPEG, Apple® QuickTime®, MP4, AVCHD®, Windows Media®, DivX™, Flash Video, Ogg Theora, WebM, and RealMedia.

In many aspects, the systems, platforms, software, networks, and methods disclosed herein include one or more non-transitory computer readable storage media encoded with a program including instructions executable by the operating system of an optionally networked digital processing device. For instance, in some aspects, the methods comprise creating data files associated with a plurality of sequencing reads from a plurality of samples associated with a food processing facility. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the systems, platforms, software, networks, and methods disclosed herein include at least one computer program. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

In some embodiments, after a first body of the 3D object is produced, the movable stage is removed from the actuator system. The 3D object may then continue to further processing steps, such as a perfusion sequence as described herein. The systems, platforms, software, networks, and methods disclosed herein include, in various embodiments, software, server, and database modules. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Definitions

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Sonoporation in a Murine Liver Model Using a Repeated Sonoporation Protocol Comparing Varying Repeat Intervals

### Experimental Animals and Protocol

There were seven experimental groups, each of which included 4 BALB/c mice, with the exception of the Baseline group which contained 3 BALB/c mice. Prior to the experiment, each mouse was implanted with a jugular vein catheter (JVC), through which the sonoactive microstructure and nucleotide constructs were administered.

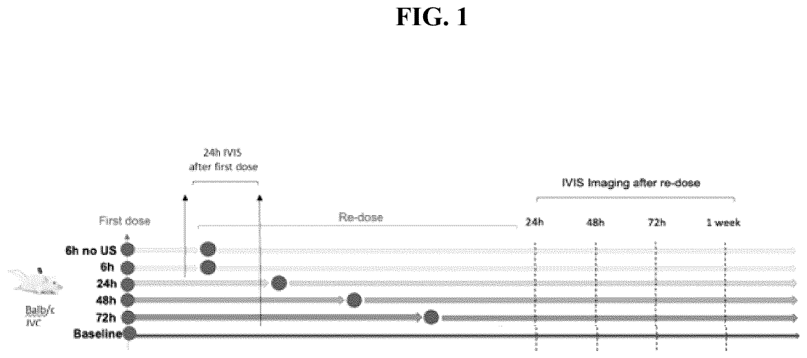

Mice in this experiment received at least one dose of a miniplasmid DNA (mpDNA) construct and sonoactive microstructure mixture. The mpDNA construct used in this experiment was a Nanoplasmid™ construct. Except in a control group, the Baseline group, all groups also received a second dose of ultrasound after the first dose was delivered. Ultrasound (US) energy was delivered transcutaneously with each dose to transfect the target organ, the liver, except in the “6 h no US” control group. In the five experimental groups, the second dose was delivered with US at either 6 h, 24 h, 48 h, or 72 h from the first dose, respectively. An illustration of these experimental protocols is shown in FIG. 1.

FIG. 1 provides illustrative experimental protocols for experiments in which two doses of a DNA construct and a sonoactive microstructure are delivered to mice in conjunction with ultrasound (two-dose sonoporation experiments). In these protocols, mice receive at least one dose of a miniplasmid DNA (mpDNA) construct and sonoactive microstructure mixture. Ultrasound, focused on a target area or target cells, is delivered to the mice with the miniplasmid DNA construct and sonoactive microstructure mixture. The miniplasmid DNA construct, for example, a nanoplasmid DNA construct, can comprise a nucleic acid payload, for example, a therapeutic transgene coupled to a promoter sequence or other regulatory element sequence.

Illustrated in FIG. 1 are multiple two-dose sonoporation protocols. Large dots along the horizontal lines represent approximate delivery times of mpDNA and sonoactive microstructure mixture doses and US. The top (“6h no US”, lightest gray) and bottom (“Baseline”, darkest gray) arrows represent control experiments in which either no ultrasound (US) is delivered to the mouse or no second dose is delivered, respectively. In other experiments, second doses are delivered at 6 h, 24 h, 48 h, or 72 h from the first dose (second from top arrow to second from bottom arrow, lighter gray to darker gray, respectively.) A dose of the nanoplasmid and sonoactive microstructure mixture comprised 250 μg of the nanoplasmid construct. The nanoplasmid construct comprised nucleic acids encoding a luciferase gene coupled to an ApoE promoter.

The period during which expression of a nucleic acid payload delivered by mpDNA, e.g., luciferase, is first measured by IVIS fluorescence imaging of mice is indicated by solid vertical arrows (24 h after dose 1). This measurement can comprise a baseline fluorescence measurement. Subsequent IVIS fluorescence imaging sessions at 24 h, 48 h, 72 h, and 1 week after the second dose (dose 2) are indicated by dashed vertical lines. IVIS fluorescence imaging sessions are performed for all mice in each group at each time point.

A dose of sonoactive microstructure and DNA solution was readied by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 225 μL of Optison into a syringe (dead space of the needle included in the calculations). With the same needle and syringe, 75 μL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space (about 50 microliters uL). Then the 18G needle is exchanged for a 25G blunt needle for injection into the JVC.

Each dose included three 100 μl doses of mpDNA and sonoactive microstructure mixture in PBS, each delivered in through the jugular vein catheter. The miniplasmid DNA construct comprised nucleic acids encoding a luciferase gene coupled to an ApoE promoter. Sonoactive microstructures, Optison™ microbubbles, were delivered to the mice at a ratio of 1:4 (mpDNA solution:sonoactive microstructure mixture, including needle dead space dispersed into mixture), by volume.

Following administration of the microbubbles and nucleic acid payload, ultrasound acoustic energy was delivered to the liver area of mice in these experiments using a L6-24 probe positioned perpendicular to the mouse to locate the lateral view of liver using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 0.8, without ceasing application of the ultrasound energy at any point during the treatment session. Nine flashes of high MI ultrasound at 0.8 were delivered with an interval of 4 seconds between each flash, and the administration of the 9 pulses was repeated three times. The high MI pulse duration was about 0.82 microseconds. The administration of the ultrasound was less than 30 seconds.

IVIS fluorescence radiance imaging of all groups was performed 24 h after the delivery of the first dose, the fluorescence measured at this time indicating the expression level of the luciferase payload, (FIG. 1, solid vertical arrows, “24 h after dose 1”). This measurement constituted a baseline fluorescence measurement. Subsequent IVIS fluorescence imaging session were performed at 24 h, 48 h, 72 h, and 1 week after the second dose (dose 2) was delivered for each group (FIG. 1, dashed vertical lines). IVIS fluorescence imaging sessions were performed for all mice in each group at each time point. Illustrated in FIG. 1 are the seven two-dose sonoporation protocols used in this experiment. Large dots along the horizontal lines represent approximate delivery times of mpDNA and sonoactive microstructure doses.

### Results

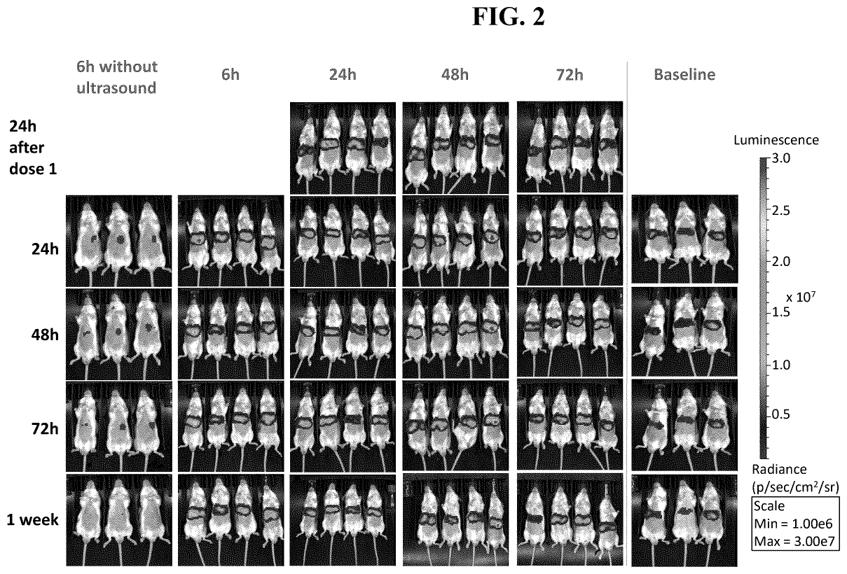

Using IVIS, mice were imaged at 24 h after the first dose (dose 1) of mpDNA and sonoactive microstructures was delivered in a first sonoporation treatment session, and then re-imaged at 24 h, 48 h, and 72 h time points after the second dose was delivered in a second sonoporation treatment session (FIG. 2) Mice in the “6 h no ultrasound” control group to which no ultrasound was delivered, had a weaker fluorescence signal at a baseline level at each measurement than did mice in groups to which ultrasound was delivered (FIG. 2, compare the left-most column to the other columns), indicating the lack of gene transfection and expression as a result of the sonoporation treatments. Mice to which no ultrasound was delivered showed very little fluorescence radiance measured at the 1 week after dose 2 time point (FIG. 2, bottom left image) indicating the lack of gene transfection and expression as a result of the lack of a sonoporation treatment. In all other groups, fluorescence radiance persisted at the 1 week after dose 2 time point (FIG. 2, bottom row), indicating that two-dose sonoporation protocols can induce durable expression of nucleic acid payloads.

FIG. 2 shows fluorescence images collected by IVIS fluorescence imaging in two-dose experiments such as those described in FIG. 1. Using IVIS, mice were imaged at 24 h after the first dose (dose 1) of mpDNA and sonoactive microstructures was delivered with US, and then re-imaged at 24 h, 48 h, 72 h, and 1 week time points after the second dose was delivered with US (shown in images in the first row to fifth row, respectively.) Five groups of mice comprised received the second dose of mpDNA and sonoactive microstructures either (1) 6 h without ultrasound, (2) 6 h with ultrasound, (3) 24 h with US, (4) 48 h with US, or (5) 72 h with US, (shown in images in the left-most to second to right-most columns, respectively). A control group comprising mice to which no second dose was delivered is shown in the right-most column (Baseline). For mice to which no ultrasound is delivered, the fluorescence signal is weaker at each time point than for mice in groups that did receive US (compare the left-most column to the other columns), indicating the efficacy of sonoporation gene transfection process. In mice that did not receive US, very little fluorescence radiance is measured at the 1 week after dose 2 time point (bottom left image). In all other mice groups, fluorescence radiance persists at the 1 week after dose 2 time point (bottom row), indicating that two-dose sonoporation protocols can induce durable expression of nucleic acid payloads.

IVIS fluorescence radiance imaging for mice in the baseline, 6 h with ultrasound, 6 h without ultrasound, 24 h, 48 h, and 72 h groups provided maximum average fluorescence radiance values for mice at the 24 h after dose 1, at 24 h, 48 h, 72 h, and 1 week time points. The mean of fluorescence radiance values for mice in each group were considered. Bar plots in FIG. 3A show the mean values for the Baseline, 6 h with US, 6 h without US, 24 h, 48 h, and 72 h groups at the 24 h, 48 h, 72 h, and 1 week after dose 2 time points. FIG. 3A represents bar plots of average fluorescence radiance measurement values for each group of n=4 mice collected by IVIS fluorescence imaging for mice in the baseline, 6 h with ultrasound, 6 h without ultrasound, 24 h, 48 h, and 72 h groups. These data show the ability of two-dose sonoporation protocols to induce durable expression of nucleic acid payloads. The values in this plot correspond to the fluorescence images shown in FIG. 2. Dots in the bar plots represent fluorescence values measured from individual mice, while the height of the bar represents the mean fluorescence value of the group at the time of measurement. Error bars represent standard deviation. Mean values 24 hours after dose 1 are also shown for the 24 h, 48 h, and 72 h groups. Individual measurements for each mouse are represented by dots. These values correspond to the fluorescence images shown in FIG. 2. Lower fluorescence values are seen in the baseline (only 1 dose delivered) and 6 h without ultrasound (2 doses delivered without ultrasound) groups than in other groups in which two doses were delivered with ultrasound. In groups that received two doses with ultrasound, high average fluorescence radiance values were measured a week after the second dose was delivered, indicating the ability of two-dose sonoporation protocols to induce durable expression of nucleic acid payloads.

FIG. 3B illustrates the results of fluorescence radiance measurements in which it is observed that subjects which underwent repeated sonoporation treatment sessions exhibited expression of the nucleic acid payload which was over an order of magnitude greater than those subjects which only underwent a single treatment session. FIG. 3B shows a line plot of average fluorescence radiance measurement values for mice in the baseline, 6 h with ultrasound, 6 h without ultrasound, 24 h, 48 h, and 72 h groups. These values correspond to the fluorescence images shown in FIG. 2 and the bar graphs shown in FIG. 3A. In accordance with the data shown in FIG. 3A, lower fluorescence values are seen in the baseline (only 1 dose delivered, indicated by an arrow labeled “single dose only/Baseline”) and 6 h without ultrasound (2 doses delivered without ultrasound, data with lowest measurements, indicated by an arrow labeled “6h without ultrasound”) groups than in other groups in which two doses were delivered with application of ultrasound. In groups that received two doses with ultrasound, high average fluorescence radiance values were measured a week after the second dose was delivered, indicating the ability of two-dose sonoporation protocols to induce durable expression of nucleic acid payloads. These data indicate the ability of two-dose sonoporation protocols to induce durable expression of nucleic acid payloads. The values in this plot correspond to the fluorescence images shown in FIG. 2 and the bar graphs shown in FIG. 3A. Markers represents the mean fluorescence value of the group at the time of measurement. Error bars represent standard deviation. The line indicating data from the Baseline group is indicated by an arrow labeled “single dose only/Baseline” and data from the 6 h without ultrasound group is indicated by an arrow labeled “6 h without ultrasound”

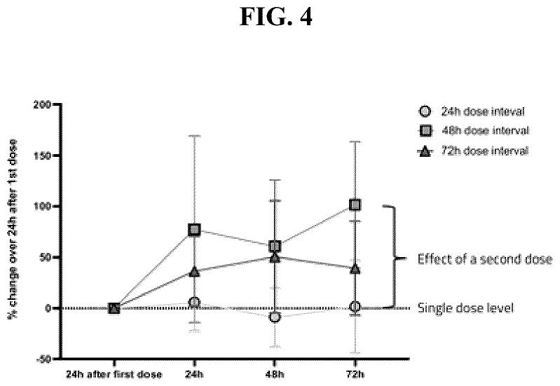

FIG. 4 shows the percent change in measured average fluorescence radiance from the 24 h after first dose measurement in three mice groups that received a second dose. These groups are: the group that received a second dose 24 hours after the first dose (circle marker), the group that received a second dose 48 hours after the first dose (square marker), and the group that received a second dose 72 hours after the first dose (triangle marker). These data suggest that two-dose sonoporation protocols in which the second dose is given more than 24 hours after the first dose may induce higher expressions levels of the nucleic acid payload. Error bars represent standard deviation. Surprisingly, it is observed that in the group that received a second dose 48 hours after the first dose (square marker) exhibited the highest level of gene expression, which persisted in the days and weeks following the sonoporation treatments, at levels one half of an order of magnitude larger (e.g., about 5-fold) than the group that received a second dose 72 hours after the first dose (diamond marker).

### Example 2: Sonoporation in a Murine Liver Model Using a Triple Repeated Sonoporation Protocol Compared to Single and Double Repeated Protocol

### Experimental Animals and Protocol

There were four experimental groups, each of which consisted of 4 BALB/c mice. Prior to the experiment, each mouse was implanted with a jugular vein catheter (JVC), through which the sonoactive microstructures and nucleic acid constructs were administered.

A dose of sonoactive microstructure and DNA solution was readied by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 225 μL of Optison into a syringe (dead space of the needle (about 50 microliters (uL)) included in the calculations). With the same needle and syringe, 75 μL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into a patent JVC.

Each dose included three 100 μl doses of mpDNA and sonoactive microstructure mixture in PBS, each delivered in through the jugular vein catheter. The miniplasmid DNA construct comprised nucleic acids encoding a luciferase gene coupled to an ApoE promoter. Sonoactive microstructures, Optison™ microbubbles or Sonazoid™ microbubbles, were delivered to the mice at a ratio of 1:4 (mpDNA solution: sonoactive microstructure mixture, including needle dead space dispersed into mixture), by volume.

Following administration of the microbubbles and nucleic acid payload, ultrasound acoustic energy was delivered to the liver area of mice in these experiments using a L6-24 probe positioned perpendicular to the mouse to locate the lateral view of liver using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 0.8, without ceasing application of the ultrasound energy at any point during the treatment session. Nine flashes of high MI ultrasound at 0.8 were delivered with an interval of 4 seconds between each flash, and the administration of the 9 pulses was repeated three times. The high MI pulse duration was about 0.82 microseconds. The administration of the ultrasound was less than 30 seconds.

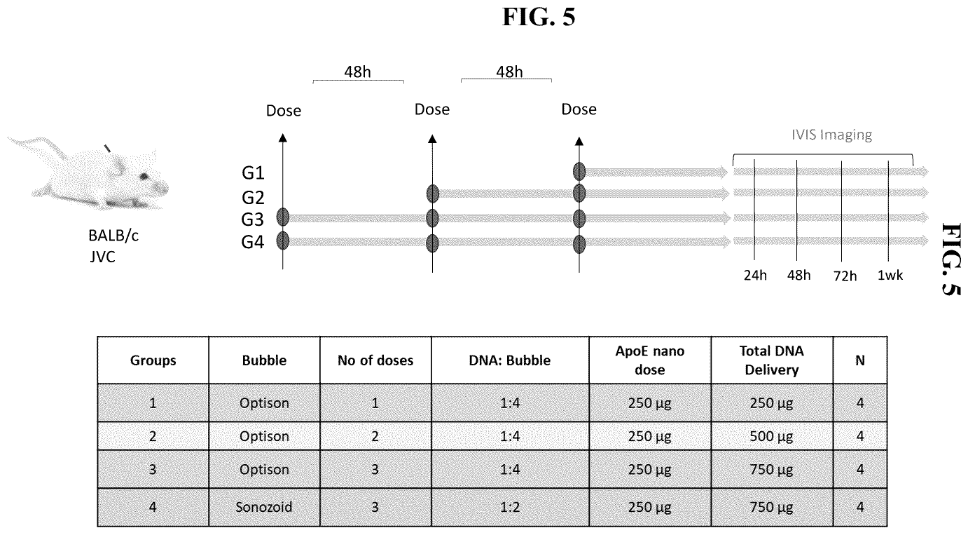

FIG. 5 provides illustrative experimental protocols for experiments in which multiple doses of a DNA construct and a sonoactive microstructure are delivered to mice in conjunction with ultrasound (multiple-dose sonoporation experiments). In these protocols, mice receive between one and three doses of a miniplasmid DNA (mpDNA) construct and sonoactive microstructure mixture. Ultrasound, focused on a target area or target cells, is delivered to the mice with the miniplasmid DNA construct and sonoactive microstructure mixture. The miniplasmid DNA construct, for example, a nanoplasmid DNA construct, can comprise a genetic payload, for example, a therapeutic transgene coupled to a promoter sequence or other regulatory element sequence.

Illustrated in FIG. 5 are four multiple-dose sonoporation protocols. Large dots along the horizontal lines represent approximate delivery times of mpDNA and sonoactive microstructure doses. The top arrow (“G1”) depicts a group in which a single 250 μg dose of mpDNA and Optison microbubble mixture at a 1:4 ratio by volume is delivered to a mouse. The second from the top arrow (“G2”) depicts a group in which two 250 μg doses of an mpDNA and Optison microbubble mixture at a 1:4 ratio by volume are delivered to a mouse with 48 h between each dose delivery. The third from the top arrow (“G3”) depicts a group in which three 250 μg doses of an mpDNA and Optison microbubble mixture at a 1:4 ratio by volume are delivered to a mouse with 48 h between each dose delivery. The fourth from the top arrow (“G4”) depicts a group in which three 250 μg doses of an mpDNA and Sonazoid microbubble mixture at a 1:2 ratio by volume are delivered to a mouse with 48 h between each dose delivery. Approximate timing of dose delivery for all groups is indicated by black vertical arrows. Expression of a genetic payload delivered by mpDNA, e.g., luciferase, is measured by IVIS fluorescence imaging of mice at 24 h, 48 h, 72 h, and 1 week after the final dose is delivered. Measurement times are indicated by black vertical lines. IVIS fluorescence imaging sessions are performed for all mice in each group at each time point.

Mice in this experiment received at least one dose of a miniplasmid DNA and sonoactive microstructure mixture. Mice in Group 1 (G1) received a single dose of a nanoplasmid and Optison microbubble mixture at a 1:4 ratio by volume. Mice in Group 2 (G2) received two doses of a nanoplasmid and Optison microbubble mixture at a 1:4 ratio by volume delivered with 48 h between each dose. Mice in Group 3 (G3) received three doses of a nanoplasmid and Optison microbubble mixture at a 1:4 ratio by volume with 48 h between each dose. Mice in Group 4 (“G4”) received three 250 μg doses of a nanoplasmid and Sonazoid microbubble mixture at a 1:2 ratio by volume delivered to a mouse with 48 h between each dose. Ultrasound (US) was applied transcutaneously with each dose to the liver. An illustration of these experimental protocols is shown in FIG. 5.

A dose of the nanoplasmid and sonoactive microstructure mixture comprised 250 μg of the nanoplasmid construct. Thus, during the experiment, a total of 250 μg of DNA was delivered to G1, 500 μg of DNA was delivered to G2, 750 μg of DNA was delivered to G3, and 750 μg of DNA was delivered to G4. The nanoplasmid construct comprised nucleic acids encoding a luciferase gene coupled to an ApoE promoter. IVIS fluorescence radiance imaging of all groups was performed 24 h, 48 h, 72 h, and 1 week, after the delivery of the last dose. IVIS fluorescence imaging sessions were performed for all mice in each group at each time point. Illustrated in FIG. 5 are the four multiple-dose sonoporation protocols used in this experiment. Large dots along the horizontal lines represent approximate delivery times of mpDNA and sonoactive microstructure doses (FIG. 5, solid vertical arrows).

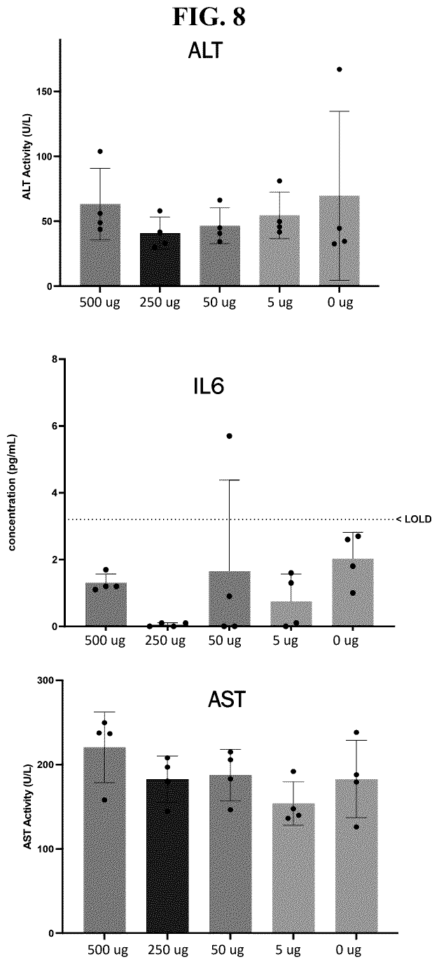

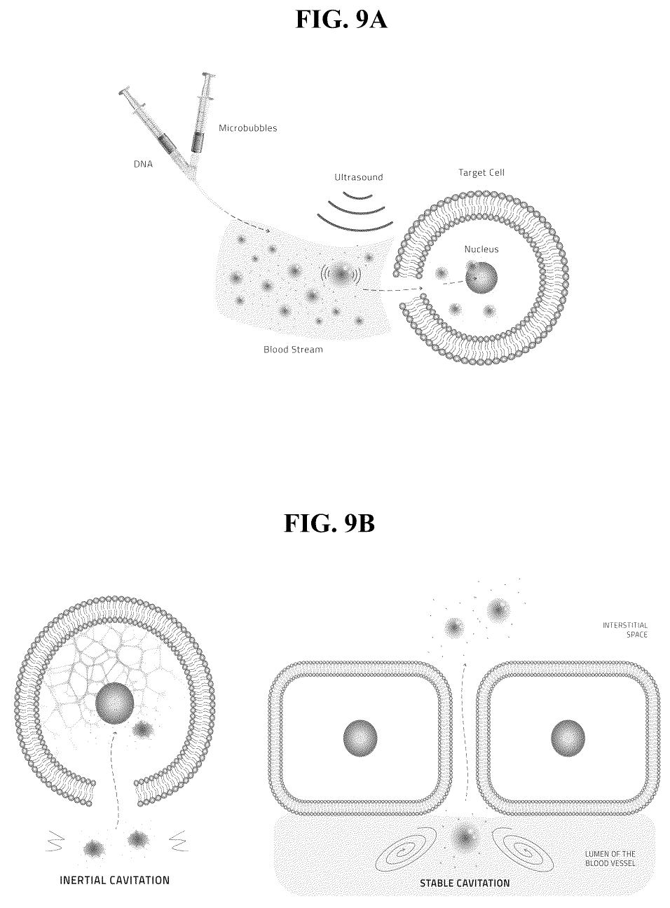

24 hours following administration of the first sonoporation treatment, serum ALT, IL6 and AST levels were measured to assess systemic inflammation, cellular inflammation, and cellular damage to the liver resulting from the sonoporation treatment. As is shown in FIG. 8, no change in liver enzymes 24 hours after delivery was observed (compare far right 0 μg bar to other bars), indicating a favorable response to the sonoporation treatment which did not result in detection of significant cellular damage to the liver or other cellular inflammatory responses.

### Results

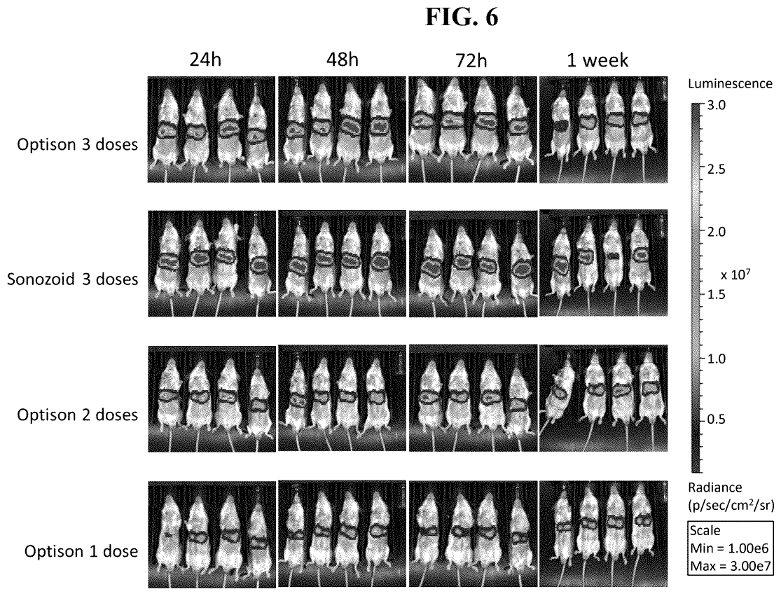

FIG. 6 shows fluorescence images collected by IVIS fluorescence imaging in multiple-dose experiments such as those described in FIG. 5. Using IVIS, mice were imaged at 24 h, 48 h, 72 h, and 1 week time points after the final dose was delivered (shown in images in the first column to seventh column, respectively.) The experimental protocol for mice in the top row (“Optison 3 doses”) corresponds to the Group 3 protocol shown in FIG. 5. In this protocol, three doses of an mpDNA and Optison microbubble mixture were administered to mice with 48 hours between each administration. The experimental protocol for mice in the second from the top row (“Sonazoid 3 doses”) corresponds to the Group 4 protocol shown in FIG. 5. In this protocol, three doses of an mpDNA and Sonazoid microbubble mixture were administered to mice with 48 hours between each administration. The experimental protocol for mice in the third from the top row (“Optison 2 doses”) corresponds to the Group 2 protocol shown in FIG. 5. In this protocol, two doses of an mpDNA and Optison microbubble mixture were administered to mice with 48 hours between each administration. The experimental protocol for mice in the bottom row (“Optison 1 dose”) corresponds to the Group 1 protocol shown in FIG. 5. In this protocol, one dose of an mpDNA and Optison microbubble mixture was administered to mice.

Using IVIS fluorescence imaging, mice were imaged at 24 h, 48 h, 72 h, and 1 week after the final dose was delivered (FIG. 6, first column to last column, respectively.) While IVIS fluorescence radiance measurements of all groups indicated expression of the luciferase genetic payload at the initial 24 h after final dose measurement to the 1 week measurement. However, mice in the groups that received either one or two doses (bottom two rows) showed longer duration of more intense luciferase expression, based on fluorescence measured by IVIS one week after the final dose was delivered. These data suggest that multiple dosing sonoporation protocols may be used to achieve long-duration expression of genetic payloads.

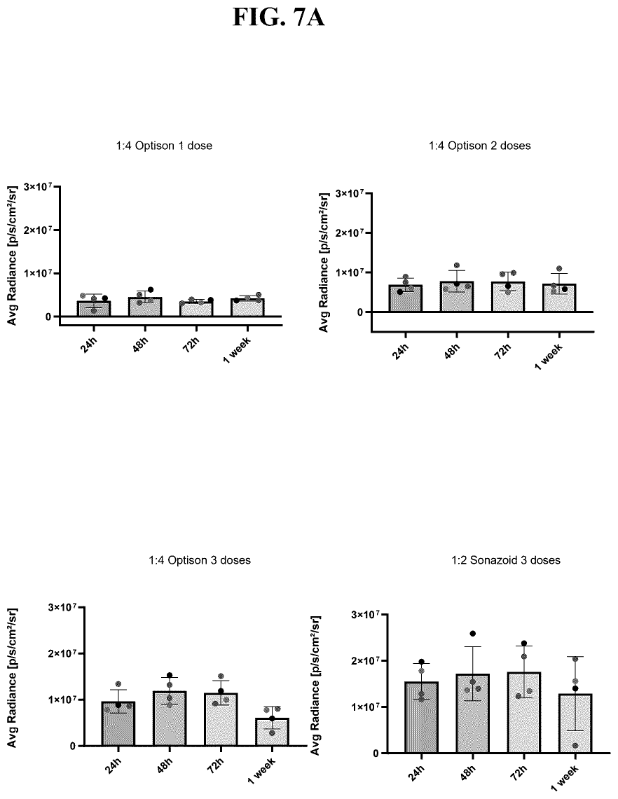

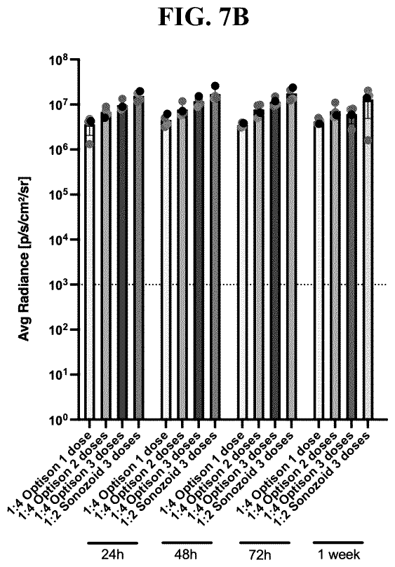

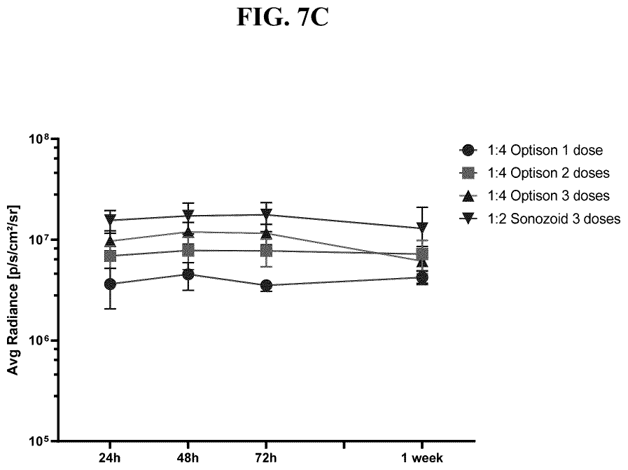

FIG. 7A represents bar plots of average fluorescence radiance measurement values collected by IVIS fluorescence imaging for mice that receive multiple doses of mpDNA and sonoactive microstructures. These values correspond to the fluorescence images shown in FIG. 6. Dots in the bar plots represent fluorescence values measured from individual mice, while the height of the bar represents the mean fluorescence value of the group at the time of measurement. Groups comprise mice that received either 1 dose (top left), 2 doses (top right), or 3 doses (bottom row). Error bars represent standard deviation. Higher initial average fluorescence radiance values are seen in the groups that received three doses (bottom row) than in the groups that received either one dose (top left) or two doses (top right). In groups that received one dose or two doses, the duration of a fluorescence signal was longer than in the groups that received three doses.

FIG. 7B represents grouped bar plots of average fluorescence radiance measurement values collected by IVIS fluorescence imaging for mice that receive multiple doses of mpDNA and sonoactive microstructures. These values correspond to the fluorescence images shown in FIG. 6 and the bar graphs in FIG. 7A. Dots in the bar plots represent fluorescence values measured from individual mice, while the height of the bar represents the mean fluorescence value of the group at the time of measurement. Groups comprise mice in which received either 1 dose (left-most bar in group, lightest gray), 2 doses (second to left-most bar in group, second to lightest gray), 3 doses with Optison microbubbles (third to left-most bar in group, third to lightest gray), or 3 doses with Sonazoid microbubbles (right-most bar in group, dark gray). Error bars represent standard deviation. Higher initial average fluorescence radiance values are seen in the groups that received three doses (right two bars in each group) than in the groups that received either one dose (left most bar in each group) or two doses (second from left bar in each group). This increase in fluorescence signal is seemingly proportional to the number of doses. In groups that received one dose or two doses, the duration of a fluorescent signal was longer than in the groups that received three doses. The duration of the fluorescence signal is also proportional to the number of doses. In early measurements, Sonazoid microbubbles showed a significantly stronger signal than did Optison microbubbles. Lower initial fluorescence values are seen at the 24 h, 48 h, and 72 h time points in the groups that received one or two doses (triangle markers) than in the groups that received three doses (circle and square markers). However, the duration of protein expression (as indicated by the presence of a fluorescence signal) was more intense for a longer in groups that received two or three doses than in groups that received one dose. These data show a positive correlation between the number of doses delivered and the initial amount of protein expression. FIG. 7C illustrates the average fluorescence radiance measurement values for each group of n=4 subjects illustrated in FIG. 6, in which sonoporation gene therapy treatments repeated in subjects resulted in an order of magnitude increase in measured fluorescence radiance.

### Example 3: Sonoporation in a NHP Model Using a Triple Repeated Sonoporation Protocol

### Experimental Animals and Protocol

There were three experimental animals, each of which was a male cynomolgus macaque. Two of the macaques were administered a nanoplasmid genetic payload and sonoactive microstructure mixture in conjunction with ultrasound (US) energy. The third macaque was naïve and did not receive any intravenous injections of microbubbles or plasmids and did not receive externally ultrasound at any time. The nucleic acid payloads utilized are summarized below.

| DNA Format | Promoter | Coding Sequence | Target organ | Dose |
|---|---|---|---|---|
| Plasmid | ApoE- | Human Factor IX | Liver | 20 mg |
| | AAT | | | |
| Nanoplasmid ™ | Ef-1a | GFP | Liver | 20 mg |
| Nanoplasmid ™ | CAG | GFP | Kidney | 20 mg |
| Nanoplasmid ™ | CAG | GFP | Heart | 20 mg |
| Nanoplasmid ™ | Ef-1a | GFP | Skeletal | 20 mg |
| | | | muscle | |

Prior to the start of an experimental session, an IV catheter was inserted into the saphenous vein. A dose of sonoactive microstructure and DNA solution was prepared by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 12 mL of Optison into a syringe (dead space of the needle included in the calculations). With the same needle and syringe, 8 mL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into the IV catheter.

Following administration of 0.5 mL mixture of the microbubbles and nucleic acid payload, ultrasound acoustic energy was delivered to the heart, kidney (e.g., unilaterally or bilaterally), liver, muscle, or a combination thereof of the subject area using an M5Sc probe positioned perpendicular to the subject. The focal depth setting was set to 3.75-4 cm, and the zoom to 0. Ultrasound was delivered continuously and alternated between a low mechanical index (MI) value of 0.09 and a high MI value of 2.3, without ceasing application of the ultrasound energy at any point during the treatment session. Nine flashes of high MI ultrasound at 2.3 were delivered approximately 20 seconds after administration of a bolus injection of sonoactive microstructure and DNA solution with an interval of about 5 seconds between each high MI flash, and the administration of the 9 pulses was repeated three times. The high MI pulse duration was about 2.25 microseconds. Subsequent bolus injections of sonoactive microstructure and DNA solution were administered approximately every 30 seconds, at which time the ultrasound probe was moved to a new location on the subject liver where ultrasound was delivered at a low mechanical index (MI) value of 0.09 to induce microbubble oscillation to and a high MI value of 2.3 induce microbubble disruption. Serial bolus injections of approximately 0.5-1.0 mL of sonoactive microstructure and DNA solution were administered to each subject about every 30 seconds until the 20 mL of DNA and microbubble solution was fully administered for that target organ, with the total treatment time occurring over about 20 minutes. The same treatment procedures was repeated at 48 hour intervals for a total of three treatment sessions per target organ.

The same procedure was repeated for each target organ (liver, kidney ×2, heart, skeletal muscle (quadricep)). For the liver, approximately 9 high MI flashes were administered at each liver lobe, including the right lobe, the left lobe, the quadrate lobe, and the caudate lobe. For the kidney, approximately 9 high MI flashes were administered at a cross sectional view, and at a longitudinal views, alternating between the views until the infusion was completed. For the skeletal muscle, the ultrasound probe was moved approximately 1 inch to a subsequent location following each bolus infusion of the sonoactive microstructure and DNA solution. Results

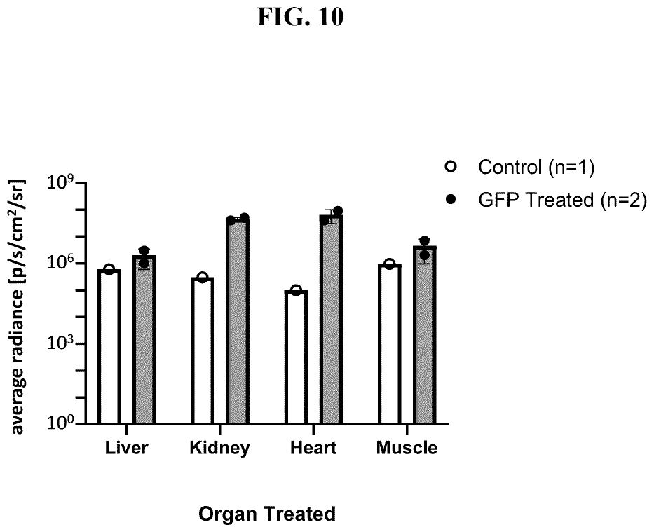

The target organs and tissues of interest were imaged. FIG. 10 shows quantitative results of fluorescence in multiple-dose experiments using non-human primate animal subjects. The kidney of NHP01 and NHP02 was observed green fluorescence color-coded yellow-orange to indicate the strength of the fluorescence signal across the entire surface of the organ, both the lateral and longitudinal cross sections. Also observed is green fluorescence color-coded yellow-orange to indicate the strength of the fluorescence signal across the peripheral boundary of the liver, across the leftmost section of the heart, and along the peripheries of the muscle.

The quantitative results of FIG. 10 show an average radiance of about 5*10\hat{ }6 p/s/cm²/sr in the liver, about 1*10\hat{ }8 p/s/cm²/sr in the kidney, about 1*10\hat{ }8 p/s/cm²/sr in the heart, and about 1*10\hat{ }7 p/s/cm²/sr in the skeletal muscle.

These data show that multiple dosing protocols can be used to induce expression of a genetic payload using ultrasound-mediated gene delivery in the kidney, liver, heart, and muscle of non-human primates.

### Example 4: Sonoporation in a Murine Liver Model Using a Triple Repeated Sonoporation Protocol Compared to Single and Double Repeated Protocol

### Experimental Animals and Protocol

There were 2 experimental groups, each of which included four RAG2 knock-out mice. Prior to the experiment, each mouse was implanted with a jugular vein catheter (JVC) through which the sonoactive microstructure and nucleic acid constructs were administered.

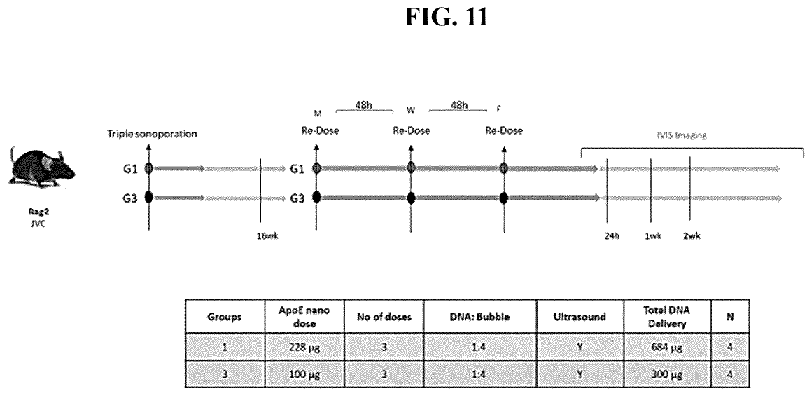

Mice in this experiment received 4 total sonoporation sessions which included three boluses of miniplasmid DNA (mpDNA) and sonoactive microstructures administered in each treatment session, with 16 weeks between the first sonoporation session and the subsequent treatment sessions, with there being three total subsequent treatment sessions starting at 16 weeks following the initial treatment session, with 48 hours between the third and the fourth treatment sessions. The mpDNA construct used in this experiment was a Nanoplasmid™ construct. Ultrasound (US) energy was delivered transcutaneously with each dose to transfect the target organ, the liver. Within a session, there were 48 hours between sonoporation treatment comprising the dose of the mpDNA construct and sonoactive microstructure mixture. An illustration of these experimental protocols is shown in FIG. 11. Group I received three doses of 228 μg of DNA payload for a total delivery of 684 μg of DNA per treatment session, and Group 3 received three doses of 100 μg of DNA payload for a total delivery of 300 μg of DNA per treatment session. The DNA payload solution to sonoactive microstructure solution volumetric ration for each group was 1:4.

A dose of sonoactive microstructure and DNA solution was prepared by first preparing the sonoactive microstructures as instructed on the label: microstructures were removed from 4C storage and rolled between the palms for 20 seconds; the protective plastic and aluminum covering from Optison vial was removed; a 25G needle was inserted through the rubber gasket of the Optison vial to provide a pressure vent; and a 1.5 inch 18G needle was used to draw up 12 mL of Optison into a syringe (dead space of the needle included in the calculations). With the same needle and syringe, either 228 μg or 100 μg of DNA payload was drawn into the syringe to combine the DNA and Optison. The Optison microbubbles and DNA payload were mixed in the syringe by rolling the syringe between the fingers until the solution was homogenous. The DNA+Optison solution was drawn out of the needle dead space. Then the 18G needle was exchanged for a 25G blunt needle for injection into the IV catheter.

Each session included either three 228 μg or three 100 μg doses of mpDNA and sonoactive microstructure mixture in PBS, each delivered through the jugular vein catheter. The miniplasmid DNA construct comprised nucleic acids encoding a luciferase gene coupled to an ApoE promoter. Sonoactive microstructures, Optison™ microbubbles, were delivered to the mice at a ratio of 1:4 (mpDNA solution: sonoactive microstructure mixture, including needle dead space dispersed into mixture), by volume.

Following administration of the microbubbles and nucleic acid payload, ultrasound acoustic energy was delivered to the liver area of mice in these experiments using a L6-24 probe positioned perpendicular to the mouse to locate the lateral view of liver using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 0.8. Nine flashes of high MI ultrasound at 0.8 were delivered with an interval of 4 seconds between each flash, and the administration of the 9 pulses was repeated three times. The high MI pulse duration was about 0.82 microseconds. The administration of the ultrasound was less than 120 seconds. Subjects were redosed in a subsequent sonoporation treatment session 16 weeks after the initial treatment session, and then at 48 hours and 96 hours following the subsequent sonoporation treatment session delivered at 16 weeks.

After the first session, IVIS fluorescence radiance imaging of all groups was performed at 24, 48, and 72 hours, and then repeated weekly for 16 weeks. After the subsequent treatment session, IVIS fluorescence radiance imaging of all groups was performed at 24, and 72 hours following the subsequent treatment sessions, and then at 1 and 2 weeks following the subsequent treatment sessions. (FIG. 11, vertical arrows). IVIS fluorescence imaging sessions were performed for all mice in each group at each time point. Large dots along the horizontal lines in FIG. 11 represent approximate delivery times of mpDNA and sonoactive microstructure doses.

### Results

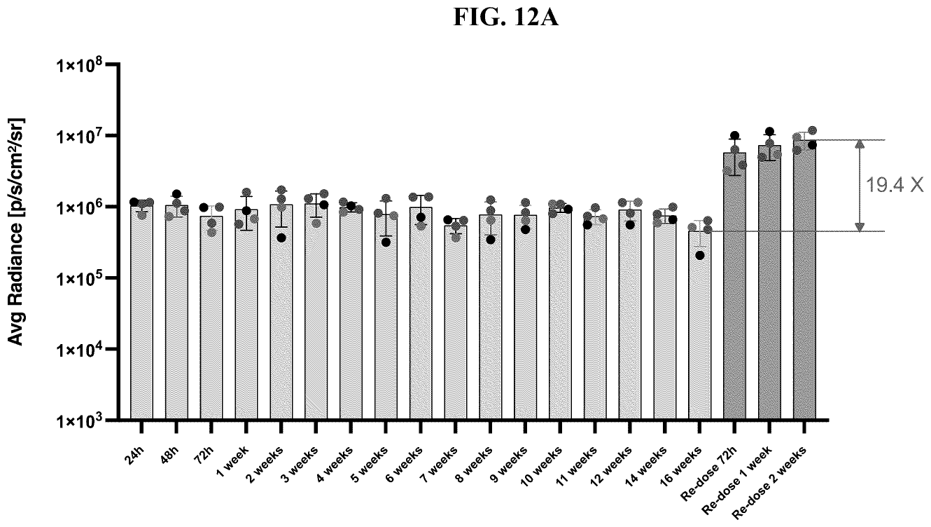

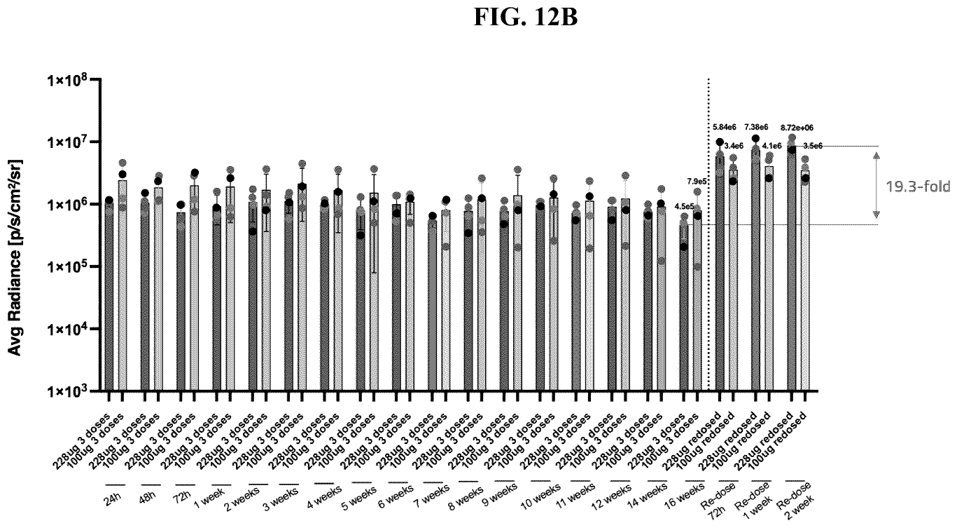

Using IVIS, after the first session, mice were imaged at 24 h, 48 h, 72 h, and then weekly after the subsequent treatment sessions were delivered. Average fluorescence radiance measured by IVIS was indicative of expression of luciferase in the target organ, the liver. Durability of expression of luciferase was observed up to 16 weeks after the first treatment session concluded (FIG. 12A and FIG. 12 B).

The average fluorescence radiance measured for mice in both groups (mice receiving either 228 μg or 100 μg of mpDNA per session) after the second session was greater than the average fluorescence radiance measured after the first session (FIG. 12A and FIG. 12B). Following the subsequent treatment sessions at 16 weeks, there was a nearly 20× increase in average fluorescence radiance measured in the group of mice that received 228 ug DNA between what was measured 16 weeks after the first session and 2 weeks after the second session (FIG. 12A and FIG. 12B). Error bars in FIG. 12A and FIG. 12B represent standard deviation. It is further observed that mice treated with either 228 ug or 100 ug mpDNA groups as described herein exhibited stable expression of luciferase for the duration of the experiment following administration of the sonoporation treatments, and a stable increased expression at the elevated expression levels following the subsequent sonoporation treatment, for at least 2 weeks.

### Example 5: Delivery of FVIII in a Murine Liver Model Using a Triple Repeated Sonoporation Protocol

Four experimental groups of 5 RAG2 mice were evaluated in this experiment, the four groups including: 1) a group administered sonoactive microstructure and 300 μg of nucleic acid constructs encoding FVIII coupled to an APOE-AAT promoter, 2) a group administered sonoactive microstructure and 750 μg of nucleic acid constructs encoding FVIII coupled to an APOE-AAT promoter, 3) a group administered sonoactive microstructure and 750 μg of nucleic acid constructs encoding FVIII coupled to an APOE-AAT promoter which did not receive ultrasound, and 4) group administered sonoactive microstructures but no nucleic acid payload and which did receive ultrasound. Prior to the experiment, each mouse was implanted with a jugular vein catheter (JVC) through which the sonoactive microstructure and nucleic acid constructs were administered.

Mice in this experiment received three total sonoporation sessions which included three boluses of miniplasmid DNA (mpDNA) encoding FVIII coupled to an APOE-AAT promoter and sonoactive microstructures administered in each treatment session, with 48 hours between the first sonoporation session and the second sonoporation sessions, and 72 hours between the second sonoporation session and the third sonoporation session. The mpDNA construct used in this experiment was a Nanoplasmid™ construct. Ultrasound (US) energy was delivered transcutaneously with each dose to transfect the target organ, the liver. Group 1 received three doses of 100 μg of DNA payload for a total delivery of 300 μg of DNA per treatment session, and Group 2 received three doses of 250 μg of DNA payload for a total delivery of 750 μg of DNA per treatment session. The DNA payload solution to sonoactive microstructure solution volumetric ration for each group was 1:4. The sonoactive microstructure utilized in this experiment was a phospholipid stabilized microbubble, Sonazoid®.

A dose of sonoactive microstructure and DNA solution was prepared by first preparing the sonoactive microstructures as instructed on the label: powder for injection is removed from the manufacturers packaging by twisting the top of the ampule, a syringe is placed directly in the ampule without using a cannula, 2 milliliters of phosphate buffered saline is added from the syringe into the vial and hand shook for one minute ensure a homogeneous product, the product is withdrawn into a syringe and reinjected back into the vial, and the vial is shaken to reconstitute the product immediately before injection and withdrawn into a syringe for injection With the same needle and syringe, either 250 μg or 100 μg of DNA payload was drawn into the syringe to combine the DNA and Sonozoid® sonoactive agent, and Sonozoid® sonoactive agent and DNA payload were mixed in the syringe by rolling the syringe between the fingers until the solution was homogenous. The DNA+Sonozoid® sonoactive agent solution was drawn out of the needle dead space. Then the 18G needle was exchanged for a 25G blunt needle for injection into the IV catheter.

Each session included either three 250 μg or three 100 μg doses of mpDNA and sonoactive microstructure mixture in PBS, each delivered through the jugular vein catheter. The miniplasmid DNA construct comprised nucleic acids encoding a FVIII gene coupled to an ApoE-AAT promoter. Sonoactive microstructures, Sonozoid® sonoactive agent, were delivered to the mice at a ratio of 1:4 (mpDNA solution: sonoactive microstructure mixture, including needle dead space dispersed into mixture), by volume.

Following administration of each bolus injection of the DNA payload and sonoactive agents, ultrasound acoustic energy was delivered to the liver area of mice in these experiments using a L6-24 probe positioned perpendicular to the mouse to locate the lateral view of liver using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 2.3. 18 flashes of high MI ultrasound at 2.3 were delivered with an interval of 4 seconds between each flash, and the administration of the 18 pulses was repeated three times. The high MI pulse duration was about 0.82 microseconds. The administration of the ultrasound was less than 120 seconds per bolus administration. Following the each bolus injection of the DNA payload and sonoactive agent, the ultrasound probe was moved to a different location on the subject liver. Subjects were redosed in a subsequent sonoporation treatment session 48 and 72 hours after the initial treatment session using the same treatment protocols.

### Results

Transgenic FVIII level in mouse plasma was measured by MSD assay. Briefly capture antibody (GMA-8024) was loaded to the 96-well plate overnight at 4 C. Next the plate was washed three times with wash buffer and incubated with blocking buffer for 30 min at room temperature. 8 point serial dilution standard were prepared using Xinta® ranging from 0.92 IU/ml to 0.01 IU/ml. 2-fold diluted samples and standards were added to the wells in 96-well plate. Incubated 2 hours at room temperature and washed 3 times. The detection was performed by incubating samples with GMA-8023 antibody during 2 hours following triple wash. Signal was developed by Sulfo-TAG and detected by MSD machine.

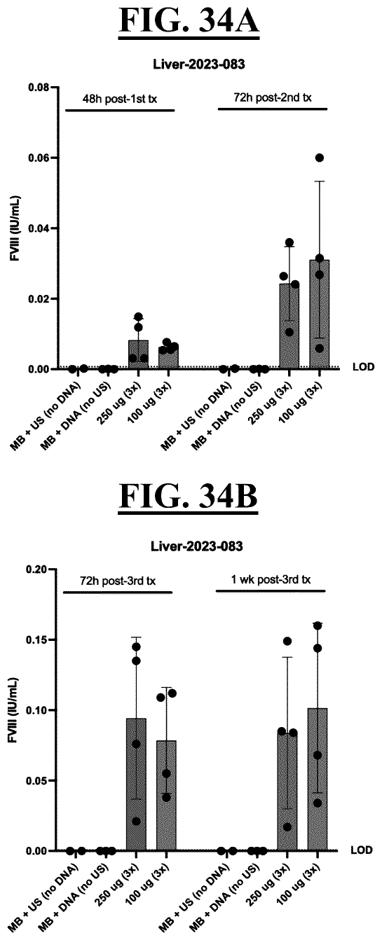

The average FVIII level for mice in the first and second (mice receiving either 300 μg or 750 μg of mpDNA per session respectively) after the first and second treatment sessions are shown in FIG. 34A, and increase (approximately triple) from about 0.01 IU/mL to about 0.03 IU/mL of FVIII upon repeating the sonoporation treatment protocol a second time. The average FVIII level measured for mice in the first and second (mice receiving either 300 μg or 750 μg of mpDNA per session respectively) 72 hours and 1 week after the third treatment sessions are shown in FIG. 34A average about 0.1 IU/ML, an increase of about 10-fold from the expression measured following the first treatment session, and about three-fold following the second treatment session. Surprisingly and unexpectedly, the results shown herein illustrate that the multiple organ location and repeated treatment protocol described herein provides significantly more than an additive effect in increasing delivery and expression of FVIII to the murine liver, with each subsequent sonoporation treatment session resulting in approximately a threefold increase in gene delivery and expression.

### Example 6: Sonoporation in a Murine Liver Model Using Measuring Dose Response

### Experimental Animals and Protocol

There were four experimental groups, each of which consisted of 4 BALB/c mice. Prior to the experiment, each mouse was implanted with a jugular vein catheter (JVC), through which the sonoactive microstructures and nucleic acid constructs were administered.

A dose of sonoactive microstructure and DNA solution was readied by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 225 μL of Optison into a syringe (dead space of the needle (about 50 microliters (uL)) included in the calculations). With the same needle and syringe, 75 μL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into a patent JVC.

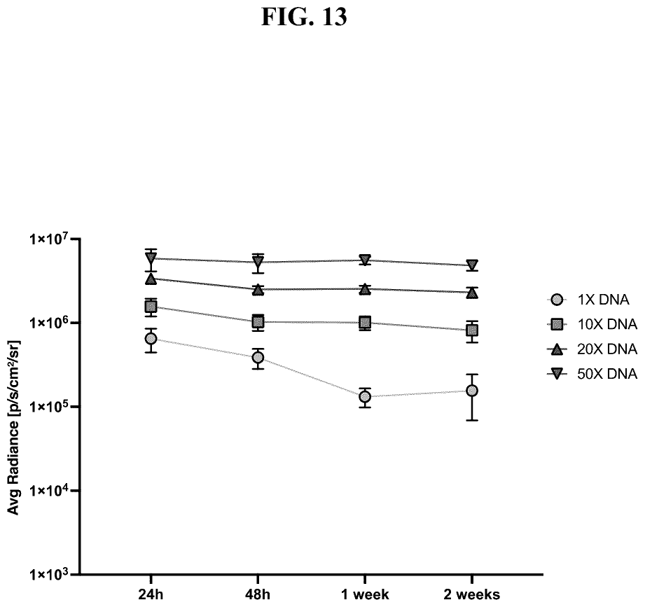

Each treatment session included three boluses of mpDNA and sonoactive microstructure mixture in PBS, totaling 200 uL, each delivered in through the jugular vein catheter. The miniplasmid DNA construct comprised nucleic acids encoding a luciferase gene coupled to an ApoE promoter. Sonoactive microstructures, Optison™ microbubbles, were delivered to the mice at a ratio of 1:4 (mpDNA solution: sonoactive microstructure mixture, including needle dead space dispersed into mixture), by volume. Mice in each of the groups were administered a dose comprising either 5 ug, 50 ug, 100 ug, or 250 μg of DNA (see 1×, 10×, 20×, and 50× DNA groups, respectively, in FIG. 13).

Following administration of the microbubbles and nucleic acid payload, ultrasound acoustic energy was delivered to the liver area of mice in these experiments using a L6-24 probe positioned perpendicular to the mouse to locate the lateral view of liver using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 0.8. Nine flashes of high MI ultrasound at 0.8 were delivered with an interval of 4 seconds between each flash, and the administration of the 9 pulses was repeated three times. The high MI pulse duration was about 0.82 microseconds. The administration of the ultrasound was less than 120 seconds.

After the administration of the sonoporation treatment and mpDNA, IVIS fluorescence radiance imaging of all groups was first performed 24 h after the delivery of the dose. Subsequent IVIS fluorescence imaging session were performed at 48 h, 1 week and 2 weeks after the dose was delivered for each group (FIG. 13). IVIS fluorescence imaging sessions were performed for all mice in each group at each time point.

### Results

Using IVIS fluorescence imaging, mice were imaged at 24 h, 48 h, 1 week and 2 weeks after the dose was delivered (FIG. 13). IVIS fluorescence radiance measurements of all groups indicated expression of the luciferase genetic payload already at 24 h after the was delivered, with the strongest fluorescence in the group that received the highest dose of DNA, and the lowest fluorescence in the group that received the lowest dose of DNA. These data suggest that there is an early positive correlation between the level of expression of the genetic payload and the dose of mpDNA delivered.

FIG. 13 represents average fluorescence radiance measurement values collected by IVIS fluorescence imaging for mice that were administered different doses of mpDNA. Error bars represent standard deviation. Higher initial average fluorescence radiance values were seen in the groups that received more mpDNA in the dose (50×DNA group and 20×DNA group) than in the groups that received less mpDNA in the dose (1×DNA group and 10×DNA group). This higher initial average fluorescence radiance measured in groups that received more DNA persisted at least until 2 weeks after dose administration. In groups that received more mpDNA, the duration and stability of a fluorescence signal was greater than in the groups that received less mpDNA. These data indicate that expression of genetic payload is titratable by modulation of the dosage of genetic material delivered.

### Example 7: Sonoporation in a Murine Kidney Model Measuring Dose Response

### Experimental Animals and Protocol

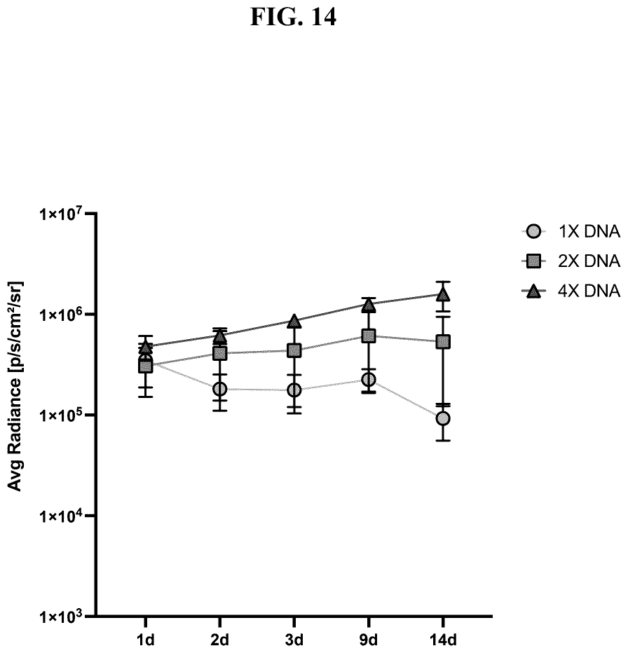

There were three experimental groups, each of which consisted of 4 BALB/c mice. Prior to the experiment, each mouse was implanted with a jugular vein catheter (JVC), through which the sonoactive microstructures and nucleic acid constructs were administered. Mice in each of the groups were administered a dose comprising either 25 ug, 50 ug, 100 μg of mpDNA (see 1×, 2×, and 4× DNA groups, respectively, in FIG. 14).

A dose of sonoactive microstructure and DNA solution was readied by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 225 μL of Optison into a syringe (dead space of the needle (about 50 microliters (uL)) included in the calculations). With the same needle and syringe, 75 μL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into a patent JVC.

Each treatment session included three boluses of mpDNA and sonoactive microstructure mixture in PBS, each delivered in through the jugular vein catheter. The miniplasmid DNA construct comprised nucleic acids encoding a luciferase gene coupled to a CAG promoter. Sonoactive microstructures, Optison™ microbubbles, were delivered to the mice at a ratio of 1:4 (mpDNA solution: sonoactive microstructure mixture, including needle dead space dispersed into mixture), by volume. Mice in each of the groups were administered a dose comprising either 25 ug, 50 ug, 100 μg of mpDNA (see 1×, 2×, and 4× DNA groups, respectively, in FIG. 14).

Following administration of the microbubbles and nucleic acid payload, ultrasound acoustic energy was delivered to the kidney area of mice in these experiments using a L6-24 probe positioned perpendicular to the mouse to locate the lateral view of kidney using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 0.8. Nine flashes of high MI ultrasound at 0.8 were delivered with an interval of 4 seconds between each flash, and the administration of the 9 pulses was repeated three times. The high MI pulse duration was about 0.82 microseconds. The administration of the ultrasound was less than 120 seconds.

After the administration of the sonoporation treatment and mpDNA, IVIS fluorescence radiance imaging of all groups was first performed 24 h after the delivery of the dose. Subsequent IVIS fluorescence imaging session were performed at 48 h, 72 h, 9 days, and 14 days after the dose was delivered for each group (FIG. 14). IVIS fluorescence imaging sessions were performed for all mice in each group at each time point.

### Results

Using IVIS fluorescence imaging, mice were imaged at 24 h, 48 h, 72 h, 9 days, and 14 days after the dose was delivered (FIG. 14). IVIS fluorescence radiance measurements of all groups indicated expression of the luciferase genetic payload already at 24 h after the was delivered, with the fluorescence measured being similar in all groups.

FIG. 14 represents average fluorescence radiance measurement values collected by IVIS fluorescence imaging for mice that were administered different doses of mpDNA. Error bars represent standard deviation. Although, the level of expression of the genetic payload was initially similar in all groups, higher average fluorescence radiance values were seen in the groups that received more mpDNA in the dose (2×DNA group and 4×DNA group) than in the group that received less mpDNA in the dose (1×DNA group) starting 48 hours after the dose was delivered. This higher average fluorescence radiance seen in groups that received more DNA persisted at least until 2 weeks after dose administration. In groups that received more mpDNA, the duration and stability of a fluorescence signal was greater than in the groups that received less mpDNA. Fluorescence signal was stronger in both the 2× and the 4× DNA groups 2 weeks after the dose was administered than it was 24 hours after the dose was administered.

### Example 8: Sonoporation in a Murine Kidney Model Utilizing a Multiple Dose and Multiple Ultrasound Location Protocol

### Experimental Animals and Protocol

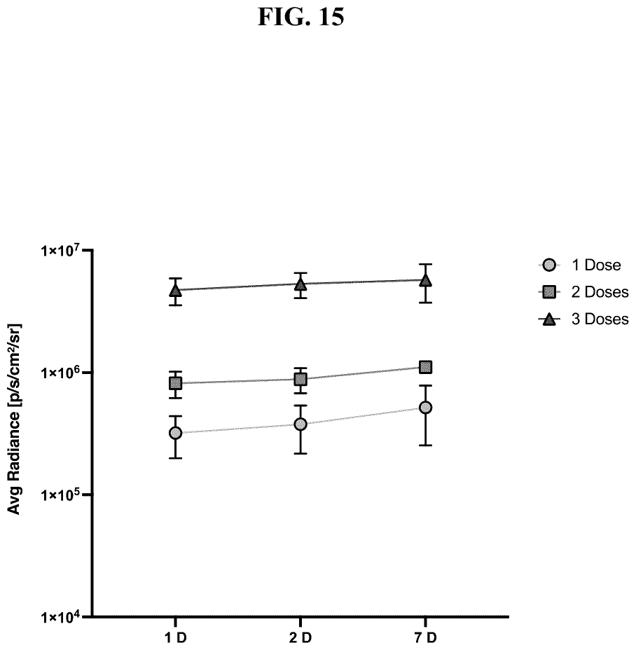

There were three experimental groups, each of which consisted of 4 BALB/c mice. Prior to the experiment, each mouse was implanted with a jugular vein catheter (JVC), through which the sonoactive microstructures and nucleic acid constructs were administered. The groups consisted of mice that were administered either 1 treatment session, 2 treatment sessions, or 3 treatment sessions of sonoactive microstructure and DNA solution (FIG. 15).

A dose of sonoactive microstructure and DNA solution was readied by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 225 μL of Optison into a syringe (dead space of the needle (about 50 microliters (uL) included in the calculations). With the same needle and syringe, 75 μL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into a patent JVC.

Each treatment session included one dose of mpDNA and sonoactive microstructure mixture in PBS, totaling 150 uL, each delivered in through the jugular vein catheter. The miniplasmid DNA construct comprised nucleic acids encoding a luciferase gene coupled to a CAG promoter. Sonoactive microstructures, Optison™ microbubbles, were delivered to the mice at a ratio of 1:4 (mpDNA solution: sonoactive microstructure mixture, including needle dead space dispersed into mixture), by volume. Mice in each of the groups were administered a dose comprising 100 μg of DNA per dose.

The groups consisted of mice that were administered either 1 treatment session, 2 treatment sessions, or 3 treatment sessions of sonoactive microstructure and DNA solution (FIG. 15). When multiple treatment sessions were administered, there was a 6 hour interval between the treatment sessions. For each treatment session, a dose of sonoactive microstructures and nucleic acid constructs were administered. Following administration of the microbubbles and nucleic acid payload, ultrasound acoustic energy was delivered to the kidney area of mice in these experiments using a L6-24 probe positioned perpendicular to the mouse to locate the lateral view of kidney using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 0.8, without ceasing application of the ultrasound energy at any point during the treatment session. Nine flashes of high MI ultrasound at 0.8 were delivered with an interval of 4 seconds between each flash, and the administration of the 9 pulses was repeated three times. The high MI pulse duration was about 0.82 microseconds. The administration of the ultrasound was less than 30 seconds.

After the administration of the sonoporation treatment and mpDNA, IVIS fluorescence radiance imaging of all groups was first performed 24 h after the delivery of the final dose. Subsequent IVIS fluorescence imaging session were performed at 48 h, and 1 week after the final dose was delivered for each group (FIG. 15). IVIS fluorescence imaging sessions were performed for all mice in each group at each time point.

### Results

Using IVIS fluorescence imaging, mice were imaged at 24 h, 48 h, and 1 week after the final dose was delivered (FIG. 15). IVIS fluorescence radiance measurements of all groups indicated expression of the luciferase genetic payload already at 24 h after the was delivered, with the strongest fluorescence in the group that received the most doses of DNA (3 dose group), and the lowest fluorescence in the group that received the fewest doses of DNA (1 dose group). These data suggest that there is a positive correlation between the level of expression of the genetic payload and the number of doses of mpDNA delivered.

FIG. 15 represents average fluorescence radiance measurement values collected by IVIS fluorescence imaging for mice that were administered different numbers of doses of mpDNA and sonoporation treatments. Error bars represent standard deviation. Higher initial average fluorescence radiance values were seen in the groups that received more doses of mpDNA in (3 doses group and 2 doses group) than in the group that received fewer doses mpDNA (1 dose group). This higher initial average fluorescence radiance measured in groups that received more doses of mpDNA persisted at least until 2 weeks after dose administration. These data indicate that expression of genetic payload is titratable by modulation of the number of doses of genetic material delivered.

### Example 9: Sonoporation in a Murine Kidney Model Utilizing a Multiple Dose and Ultrasound Treatment Location Protocol

### Experimental Animals and Protocol

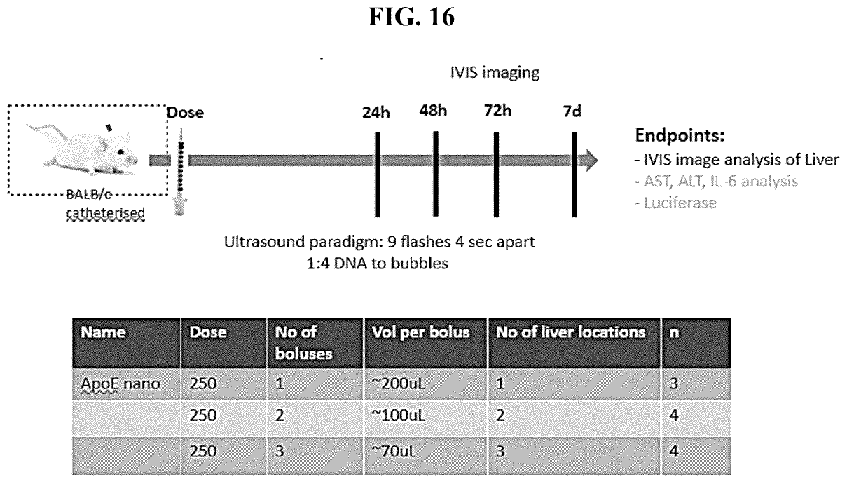

Three groups of BALB/c mice were administered sonoactive microstructures and nanoplasmid encoding a luciferase, delivered intravenously through a peripheral vein catheter. Mice were administered one, two, or three doses (“boluses”) of a DNA payload (nanoplasmid) and sonoactive microstructures (“microbubbles) in a treatment session, with groups receiving multiple doses of the DNA payload receiving application of ultrasound to a subsequent location on the kidney following administration of the subsequent dose. Microbubbles were administered at a concentration of 5*10\hat{ }8 and about 8*10\hat{ }8 microbubbles/mL. Each group was administered a total of 250 μg of nanoplasmid in solution mixed with a solution of sonoactive microstructures spread across either one, two, or three doses, over a period of 15 seconds. Group 1 was administered 250 μg of nanoplasmid and microbubble solution in a single bolus injection and received a total injectate volume of about 200 uL. Group 2 was administered 250 μg of nanoplasmid and microbubble solution in two bolus injections and received two intravenous injections of about 100 uL. Group 3 was administered 250 μg of nanoplasmid and microbubble solution in two bolus injections and received three intravenous injections of about 100 uL. After each administration, continuous ultrasound energy was administered transcutaneously to the liver at either one location for the one bolus group, two locations for the two bolus group, or three locations for the three bolus group. Each ultrasound administration for each bolus administration comprised 9 ultrasound pulses at 4 second intervals. Luciferase expression was measured by IVIS 1, 2, 5, 7, or 14 days after administration. FIG. 16 provides an illustrative protocol described in this Example.

### Results

Using IVIS, mice were imaged 1, 2, 5, 7, or 14 days after administration of the nanoplasmid, microbubbles, and ultrasound energy. FIG. 17 shows the raw IVIS images from mice receiving one bolus and ultrasound energy at one location (left column), two boluses and ultrasound energy two locations (center column), and three boluses and ultrasound energy at three location (right column) at the indicated time points.

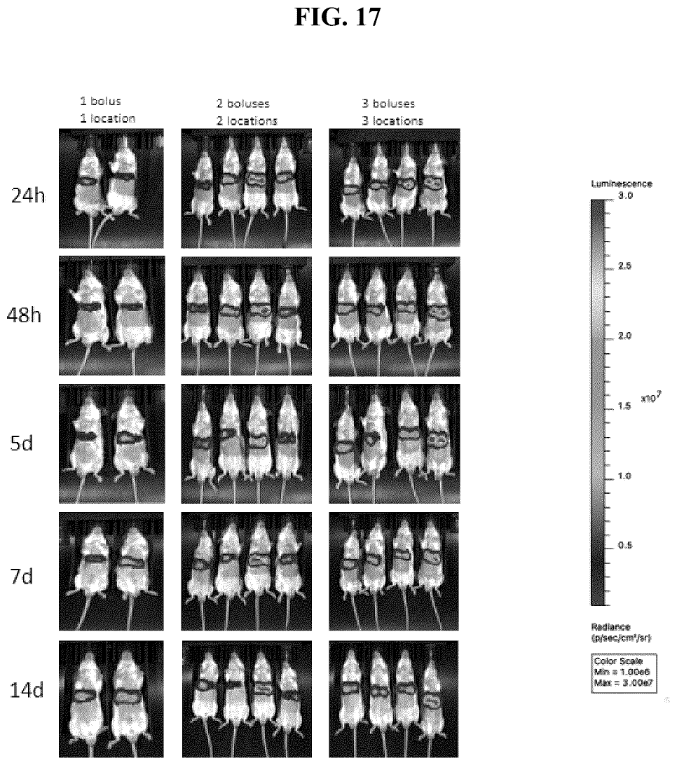

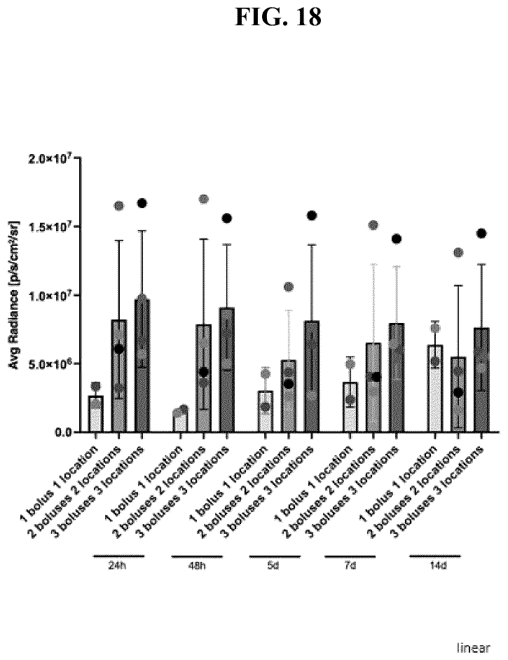

As shown in FIGS. 17 and 18, expression of luciferase is observed in all three experimental groups 24 hours after administration and persists for at least 14 days. For the first week post-administration, expression levels appear to correlate with increased number of bolus injections of what is otherwise a same dosage of DNA payload, with highest average expression levels observed in the experimental group receiving three bolus injections and ultrasound energy at three locations, and lowest expression levels in mice receiving one dose and ultrasound energy at one location (FIG. 18). Expression is still detected in all experimental groups 14 days post-administration.

### Example 12: Sonoporation in a NHP Kidney Model Utilizing a Multiple Bolus and Multiple Ultrasound Location Protocol

### Animals and Protocol

There were three experimental animals, each of which was a male cynomolgus macaque. Two of the macaques were administered a nanoplasmid genetic payload and sonoactive microstructure mixture in conjunction with ultrasound (US) energy. The third macaque was naïve and did not receive any intravenous injections of microbubbles or plasmids and did not receive externally ultrasound at any time. The nucleic acid payloads utilized are summarized below.

| DNA Format | Promoter | Coding Sequence | Target organ | Dose |
|---|---|---|---|---|
| Nanoplasmid ™ | CAG | GFP | Kidney | 20 mg |

Prior to the start of an experimental session, an IV catheter was inserted into the saphenous vein. A dose of sonoactive microstructure and DNA solution was prepared by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 12 mL of Optison into a syringe (dead space of the needle included in the calculations). With the same needle and syringe, 8 mL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into the IV catheter.

Following administration of 0.5 mL mixture of the microbubbles and nucleic acid payload, ultrasound acoustic energy was delivered to the kidney (e.g., unilaterally or bilaterally), of the subject area using an M5Sc probe positioned perpendicular to the subject. The focal depth setting was set to 3.75-4 cm, and the zoom to 0. Ultrasound was delivered continuously and alternated between a low mechanical index (MI) value of 0.09 and a high MI value of 2.3, without ceasing application of the ultrasound energy at any point during the treatment session. Nine flashes of high MI ultrasound at 2.3 were delivered approximately 20 seconds after administration of a bolus injection of sonoactive microstructure and DNA solution with an interval of about 5 seconds between each high MI flash, and the administration of the 9 pulses was repeated three times. The high MI pulse duration was about 2.25 microseconds. Subsequent bolus injections of sonoactive microstructure and DNA solution were administered approximately every 30 seconds, approximately 9 high MI flashes were administered at a cross sectional view, and at a longitudinal views, alternating between the views until the infusion was completed. The same treatment procedures were repeated at 48 hour intervals for a total of three treatment sessions per kidney.

At the end of the experiment, the NHP kidneys were sectioned. Two sections from the kidney of each experimental animal and one section from the kidney of the naïve animal were analyzed using RNAscope to assess transfection of kidney cells using ultrasound-mediated gene delivery (UMGD).

Kidney samples were analyzed using an RNAscope™ assay (RNAscope™ 2.5 LSx Red Reagent Kit; Cat. No. 322750). Based on assessment of tissue samples with reference positive and negative control probes, optimal pretreatment conditions for samples were established to maximize signal-to-noise ratio. Standard RNAscope™ 2.5 LSx Red Assay pretreatment conditions were used as follows: (i) epitope retrieval 2:15 minutes at 95° C. Protease III: 15 minutes at 40° C.

Control probes used were: (i) ACD positive control probe (Macaca fascicularis, peptidylprlul Isomerase B (Cyclophilin B); Mfa-PPIB; Cat. No. 424148); ACD negative control probe (Bacillus subtilis dihydrodipicolinate reductase; dapB; Cat. No. 312038). The target probe was (Enhanced green fluorescent protein (EGFP), mRNA; EGFP-04; Cat. No. 538858; GenBack Accession Number MN623123.1). These probes are summarized below.

| Control Probe Info | Probe Name | Probe Symbol | Catalog Number |
|---|---|---|---|
| ACD Positive Control Probe | Macaca fascicularis Peptidylprolyl | Mfa-PPIB | 424148 |
| | Isomerase B (Cyclophilin B) | | |
| ACD Negative Control Probe | Bacillus subtilis dihydrodipicolinate | dapB | 312038 |
| | reductase | | |

| Catalog | | Probe | Probe GenBank | # of ZZ | Gene Region Probes |
|---|---|---|---|---|---|
| Number | Probe Name | Symbol | Accession Number | Probe Pairs | Designed Against |
| 538858 | Mycobacterium tuberculosis | EGFP-O4 | MN623123.1 | 18 | 1166-1885 nt |
| | H37Rv plasmid pTYGi9 | | | | |

Visual scoring was performed to assign a single score to a sample based on the predominant staining pattern throughout the entire sample. Intensity of the stain did not have an impact on scoring. Dots in histological images correlate to the number of individual RNA molecule. Percentage of cells positive was scored visual based on the number of cells with more than 1 dot per cell and binned into categories (i.e., 0%, 1-25%, 26-25%, 51-75%, 76-99%, 100%).

### Results

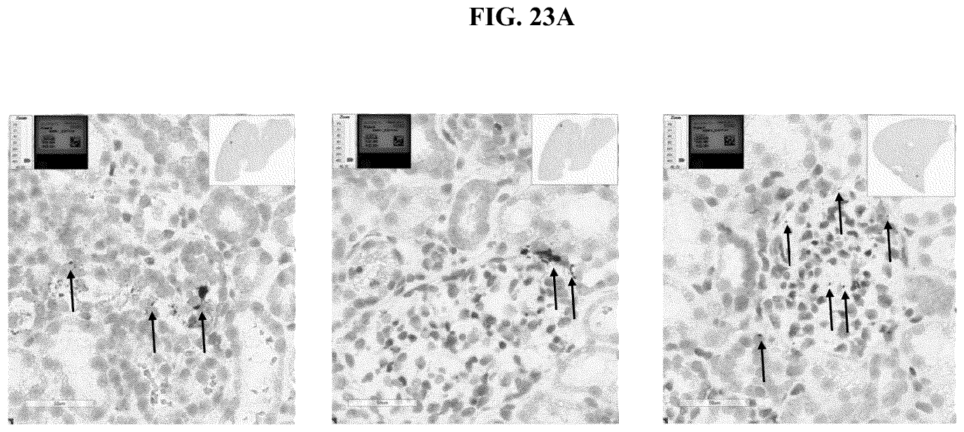

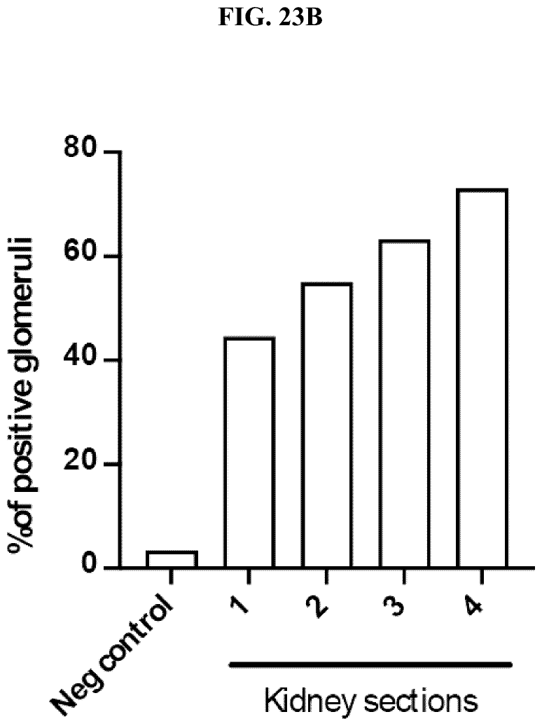

Five kidney samples were evaluated by RNAscope™ 2.5 LSx Red. All samples passed a quality control check with moderate PPIB positive control staining and little to no dapB background staining. RNAscope™ 2.5 LSx Red Assay was performed in all samples to evaluate EGFP expression in NHP kidney samples. EGFP was detected in all four kidney samples from the experimental animals and no EGFP was detected in the kidney sample of the naïve animal. Examples of histological images of the samples are provided in FIG. 23A (arrows indicate EGFP positive cells). FIG. 23B provides the percent of glomeruli positive for EGFP in kidney sections of NHP. Minimal EGFP expression was detected in the sample from the naive NHP (negative control), while expression of EGFP was detected in over 70% of glomeruli in one experimental sample. These results indicate that UMGD is effective in delivering a genetic payload to the NHP kidney, and that the repeated sonoporation with multiple organ location protocol effectively delivers and drives expression of a genetic payload in the NHP kidney.

### Example 13: Sonoporation in a NHP Kidney Model Utilizing a Multiple Bolus and Multiple Ultrasound Location Protocol Showing Delivery to Glomerular and Non-Glomerular Areas

### Animals and Protocol

There were three experimental animals, each of which was a male cynomolgus macaque. Two of the macaques were administered a nanoplasmid genetic payload and sonoactive microstructure mixture in conjunction with ultrasound (US) energy. The third macaque was naïve and did not receive any intravenous injections of microbubbles or plasmids and did not receive externally ultrasound at any time. The nucleic acid payloads utilized are summarized below.

| DNA Format | Promoter | Coding Sequence | Target organ | Dose |
|---|---|---|---|---|
| Nanoplasmid ™ | CAG | GFP | Kidney | 20 mg |

Prior to the start of an experimental session, an IV catheter was inserted into the saphenous vein. A dose of sonoactive microstructure and DNA solution was prepared by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 12 mL of Optison into a syringe (dead space of the needle included in the calculations). With the same needle and syringe, 8 mL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into the IV catheter.

Following administration of 0.5 mL mixture of the microbubbles and nucleic acid payload, ultrasound acoustic energy was delivered to the kidney (e.g., unilaterally or bilaterally), of the subject area using an M5Sc probe positioned perpendicular to the subject. The focal depth setting was set to 3.75-4 cm, and the zoom to 0. Ultrasound was delivered continuously and alternated between a low mechanical index (MI) value of 0.09 and a high MI value of 2.3, without ceasing application of the ultrasound energy at any point during the treatment session. Nine flashes of high MI ultrasound at 2.3 were delivered approximately 20 seconds after administration of a bolus injection of sonoactive microstructure and DNA solution with an interval of about 5 seconds between each high MI flash, and the administration of the 9 pulses was repeated three times. The high MI pulse duration was about 2.25 microseconds. Subsequent bolus injections of sonoactive microstructure and DNA solution were administered approximately every 30 seconds, approximately 9 high MI flashes were administered at a cross sectional view, and at a longitudinal views, alternating between the views until the infusion was completed. The same treatment procedures were repeated at 48 hour intervals for a total of three treatment sessions per kidney.

At the end of the experiment, the NHP kidneys were sectioned. One section from the kidney of each experimental animal and one section from the kidney of the naïve animal were analyzed using RNAscope to assess transfection of specific kidney cell types in glomeruli and non-glomeruli areas using ultrasound-mediated gene delivery (UMGD).

Kidney samples were analyzed using an RNAscope™ assay (RNAscope™ 25 LS Multiplex Fluorescent ISH Reagent Kit; Cat. No. 322800; and RNAscope™ LS 4-Plex Ancillary Kit for Multiplex Fluorescent ISH Reagent Kit; Cat. No. 322830). Based on assessment of tissue samples with reference positive and negative control probes, optimal pretreatment conditions for samples were established to maximize signal-to-noise ratio. Standard RNAscope™ Multiplex FL Assay pretreatment conditions were used as follows: (i) epitope retrieval 2:15 minutes at 95° C. and Protease III: 15 minutes at 40° C.

Target probes were: (1) Enhanced green fluorescent protein (EGFP), mRNA; EGFP-04; Cat. No. 538858; GenBack Accession Number MN623123.1; (2) Macaca fascicularis nephrosis 2 podocin (NPHS2), mRNA; Mfa-NPHS2-C2; Cat. No. 870598-C2; GenBack Accession Number NM_005540114.2; (3) Macaca fascicularis platlet/endothelial cell adhesion molecule 1 (PECAM1), mRNA; Mfa-PECAM1-C1-C3; Cat. No. 434498-C3; GenBack Accession Number NM_005584700.1; and (4) Homo sapiens channel nonvoltage-gated 1 alpha (SCNN1A), mRNA; Hs-SCNN1A-C4; Cat. No. 480978-C4; GenBack Accession Number NM_001038.5. These probes are summarized below.

| Catalog | | | Probe GenBank | # of ZZ | Gene Region Probes |
|---|---|---|---|---|---|
| Number | Probe Name | Probe Symbol | Accession Number | Probe Pairs | Designed Against |
| 538858 | Enhanced green fluorescent protein | EGFP-O4 | MN623123.1 | 18 | 1166-1885 nt |
| | (EGFP), mRNA | | | | |
| 870598-C2 | Macaca fascicularis nephrosis 2 | Mfa-NPHS2-C2 | NM_005540114.2 | 20 | 547-1561 nt |
| | podocin, NPHS2), mRNA | | | | |
| 434498-C3 | Macaca fascicularis platlet/endothelial | Mfa-PECAM1-C1-C3 | NM_005584700.1 | 20 | 734-1696 nt |
| | cell adhesion molecule 1 (PECAM1), | | | | |
| | mRNA | | | | |
| 480978-C4 | Homo sapiens channel nonvoltage- | Hs-SCNN1A-C4 | NM_001038.5 | 20 | 946-2025 nt |
| | gated 1 alpha (SCNN1A), mRNA | | | | |

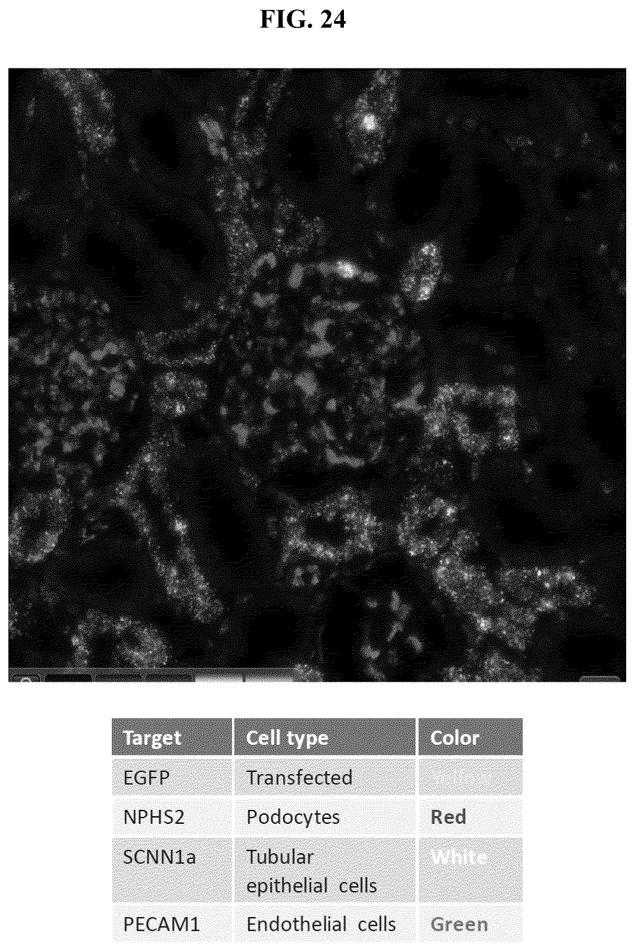

The target probes indicated: (1) EGFP: payload expression in kidney cells; (2) NPHS2: podocytes; (3) SCNN1a: tubular epithelial cells; and (4) PECAM1: endothelial cells (FIG. 24).

Visual scoring was performed to assign a single score to a sample based on the predominant staining pattern throughout the entire sample. Intensity of the stain did not have an impact on scoring. Dots in histological images correlate to the number of individual RNA molecule. Percentage of cells positive was scored visual based on the number of cells with more than 1 dot per cell and binned into categories (i.e., 0%, 1-25%, 26-25%, 51-75%, 76-99%, 100%)

### Results

Three NHP kidney samples were evaluated by RNAscope™ 25 LS Multiplex Fluorescent ISH. All samples had previously passed a quality control check (see Example 12). RNAscope™ 25 LS Multiplex Fluorescent ISH was performed to evaluate the expression of EGFP-04 mRNA along with the expression of NPHS2, SCNN1A, and PECAM1 mRNAs in the three kidney samples. EGFP-04 expression indicated the expression of the genetic payload in the kidney cells. SCNN1A-positive cells indicated a tubular epithelial cell type. NPHS2-positive cells indicated a podocyte cell type. PECAM1 expression indicated an endothelial cell type. An example histological section is provided in FIG. 24. EGFP-04 mRNA expression was observed in both treated kidney samples and no detection was observed in the naïve sample. NPHS2, SCNN1A and PECAM1 mRNA expression were also observed throughout all kidney samples. EGFP-04 mRNA expression was observed in all treated samples and absent in the naïve sample. The various cell markers were also observed in all samples.

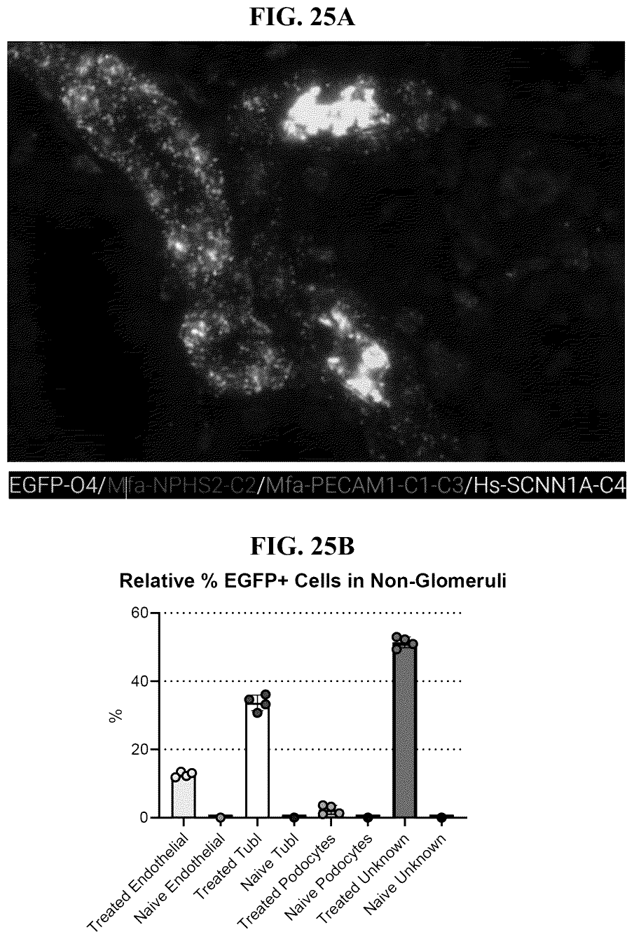

FIG. 25A provides an example histological sample with staining for EGFP, NPHS2, PECAM1, and SCNN1A in a non-glomeruli area of the NHP kidney. FIG. 25B provides a quantitation of the percent of endothelial (PECAM1-positive), Tubular epithelial (SCNN1A-positive), and podocyte (NPHS2-positive) cells that were also EGFP-04 positive in treated and naïve samples in the non-glomeruli area. EGFP-04 expression was minimal in all three cell types in the naïve animal, while all three cell types expressed EGFP-04 in the treated animals, with the most expression seen in tubular cells in the non-glomeruli area.

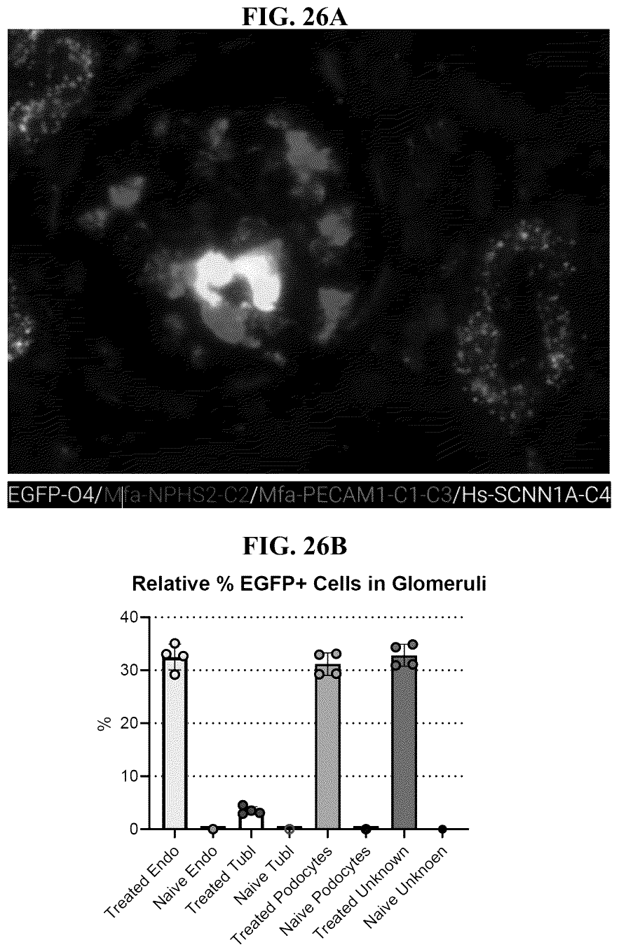

FIG. 26A provides an example histological sample with staining for EGFP, NPHS2, PECAM1, and SCNN1A in a glomeruli area of the NHP kidney. FIG. 26B provides a quantitation of the percent of endothelial (PECAM1-positive), Tubular epithelial (SCNN1A-positive), podocyte (NPHS2-positive), and unknown cells that were also EGFP-04 positive in treated and naïve samples in the glomeruli area. EGFP-04 expression was minimal in all three cell types in the naïve animal, while all three cell types expressed EGFP-04 in the treated animals, with the most expression seen in endothelial cells and podocytes in the glomeruli area.

These results indicate that UMGD is effective in delivering a genetic payload to the NHP kidney in endothelial, tubular epithelial, and podocyte cells in glomeruli and non-glomeruli areas, and that the repeated sonoporation with multiple organ location protocol effectively delivers and drives expression of a genetic payload in the NHP kidney.

### Example 14: Sonoporation in a Murine Kidney Model Utilizing a Multiple Bolus and Multiple Ultrasound Location Protocol Showing Delivery to Glomerular and Non-Glomerular Areas

### Animals and Protocol

There were two experimental groups of Balb/C mice, one with two animals administered a nanoplasmid genetic payload and sonoactive microstructure mixture in conjunction with ultrasound (US) energy. The second experimental group included naïve mouse and did not receive any intravenous injections of microbubbles or plasmids and did not receive application of ultrasound at any time. Prior to the experiment, the treated mice was implanted with a jugular vein catheter (JVC), through which the sonoactive microstructures and nucleic acid constructs were administered.

A dose of sonoactive microstructure and DNA solution was readied by first preparing the sonoactive microstructures as instructed on the label: activate DEFINITY RT by shaking the vial for 45 seconds, after activation, but no more than 15 minutes, place the activated DEFINITY RT vial in the upright position, injection of 1.4 mL of PBS into the vial, rapidly swirl the upright vial for 10 seconds to mix the contents, and using a; and using 1.5 inch 18G needle to draw up 150 μL of Definity RT microbubbles (phospholipid stabilized) into a syringe. With the same needle and syringe, 50 μL of DNA payload was drawn into the syringe to combine the DNA and DEFINITY RT. The DEFINITY RT microbubbles and DNA payload were mixed in the syringe by rolling the syringe between the fingers until the solution is homogenous. The DNA+DEFINITY RT solution was drawn out of the needle dead space. Then the 18G needle was exchanged for a 25G blunt needle for injection into a patent JVC.

Each treatment session included three doses of mpDNA (Nanoplasmid) administered as bolus injections and sonoactive microstructure mixture in PBS, each delivered in through the jugular vein catheter. Each dose of mpDNA was 275 μg (116 u mol), and in total 825 μg (348 u mol) of mpDNA was delivered in the treatment session. Following administration of each bolus injections, the ultrasound probe was moved from a cross sectional view to a longitudinal view (e.g., rotating about the mouse about 90 degrees), and alternating between the views until the infusion was completed. The miniplasmid DNA construct comprised nucleic acids encoding a luciferase gene coupled to a CAG promoter (Np-CAG-Fluc).

Mice were administered 3 doses as three bolus injections. For each dose, following administration of the microbubbles and nucleic acid payload, ultrasound acoustic energy was delivered to the kidney area of mice in these experiments using a L6-24 probe using ultrasound imaging at the low mechanical index (MI) value of 0.09. Ultrasound was delivered continuously and alternated between a low mechanical index (MI) value of 0.07 and a high MI value of 0.8, without ceasing application of the ultrasound energy at any point during the treatment session. Nine flashes of high MI ultrasound at 0.8 were delivered with an interval of 2 seconds between each flash, and the administration of the 9 pulses was repeated three times. The high MI pulse duration was about 0.82 microseconds. The administration of the ultrasound was less than 30 seconds per bolus injection.

At the end of the experiment, the right and left kidneys of the treated and untreated mice were sectioned. Two section from the kidney of the treated animal and one section from the kidney of the naïve animal were analyzed using RNAscope to assess transfection of specific kidney cell types in glomeruli and non-glomeruli areas using ultrasound-mediated gene delivery (UMGD).

Kidney samples were analyzed using an RNAscope™ assay (RNAscope™ 25 LS Multiplex Fluorescent ISH Reagent Kit; Cat. No. 322800; and RNAscope™ LS 4-Plex Ancillary Kit for Multiplex Fluorescent ISH Reagent Kit; Cat. No. 322830). Based on assessment of tissue samples with reference positive and negative control probes, optimal pretreatment conditions for samples were established to maximize signal-to-noise ratio. Standard RNAscope™ Multiplex FL Assay pretreatment conditions were used as follows: (i) epitope retrieval 2:15 minutes at 95° C. and Protease III: 15 minutes at 40° C.

Positive and negative control probes are provided below.

| Control Probe Info | Probe Name | Probe Symbol | Catalog Number |
|---|---|---|---|
| ACD Positive Control Probe | Mus musculus Peptidylprolyl Isomerase B | Mm-Ppib-C1 | 313918-C2 |
| | (Cyclophilin B) | | |
| ACD Positive Control Probe | Mus musculus Polymerase (RNA) II | Mm-Polr2a-C2 | 312478-C3 |
| | (DNA directed) polypeptide A (POLR2A) | | |
| ACD Positive Control Probe | Mus musculus Ubiquitin C (UBC) | Mm-Ubc-C3 | 310778-C3 |
| ACD Positive Control Probe | Mus musculus Hypoxanthine | Mm-Hprt-C4 | 312958-C4 |
| | Phosphoribosyltransferase | | |
| ACD Negative Control Probe | Bascillus subtilis gene dihydrodipicolinate | dapB-C1/dapB-C2/ | 312038 |
| | reductase | dapB-C3/dapB-C4 | |

Target probes were: (1) Enhanced green fluorescent protein (EGFP), mRNA; EGFP-04; Cat. No. 538858; GenBack Accession Number MN623123.1; (2) Mus musculus nephrosis 2 podocin (NPHS2), mRNA; Mm-NPHS2-C2; Cat. No. 507058-C2; GenBack Accession Number NM_130456.4; (3) Mus musculus platlet/endothelial cell adhesion molecule 1 (PECAM1), mRNA; Mm-PECAM1-C4; Cat. No. 316728-C4; GenBack Accession Number NM_001032378.1; and (4) Mus musculus channel nonvoltage-gated 1 alpha (SCNN1A), mRNA; Mm-SCNN1A-C3; Cat. No. 441398-C3; GenBack Accession Number NM 011324.2.

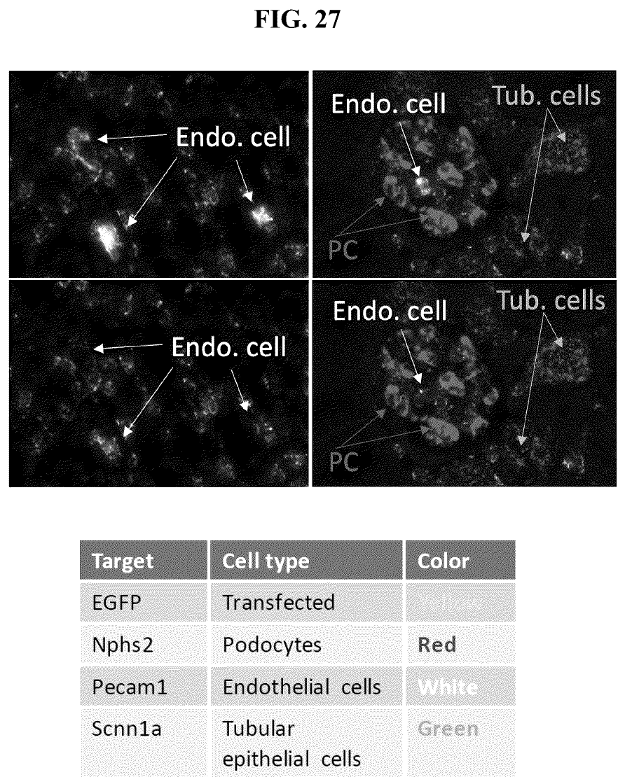

The target probes indicated: (1) EGFP: payload expression in kidney cells; (2) NPHS2: podocytes; (3) SCNN1a: tubular epithelial cells; and (4) PECAM1: endothelial cells (FIG. 27). These probes are summarized below.

| Catalog | | | Probe GenBank | # of ZZ | Gene Region Probes |
|---|---|---|---|---|---|
| Number | Probe Name | Probe Symbol | Accession Number | Probe Pairs | Designed Against |
| 538858 | Enhanced green fluorescent protein | EGFP-O4 | MN623123.1 | 18 | 1166-1885 nt |
| | (EGFP), mRNA | | | | |
| 507058-C2 | Mus musculus nephrosis 2 podocin, | Mm-Nphs2-C2 | NM_130456.4 | 20 | 266-1226 nt |
| | NPHS2), mRNA | | | | |
| 441398-C3 | Mus musculus channel nonvoltage-gated 1 | Mm-Scnn1a-C3 | NM_011324.2 | 20 | 1290-2323 nt |
| | alpha (SCNN1A), mRNA | | | | |
| 316728-C4 | Mus musculus platlet/endothelial cell | Mm-Pecam1-C4 | NM_001032378.1 | 20 | 915-1827 nt |
| | adhesion molecule 1 (PECAM1), mRNA | | | | |

Visual scoring was performed to assign a single score to a sample based on the predominant staining pattern throughout the entire sample. Intensity of the stain did not have an impact on scoring. Dots in histological images correlate to the number of individual RNA molecules. Percentage of cells positive was scored visual based on the number of cells with more than 1 dot per cell and binned into categories (i.e., 0%, 1-25%, 26-25%, 51-75%, 76-99%, 100%).

Single nucleus RNA sequencing (snRNAseq) was also performed on the kidney samples to profile gene expression in the kidney cells in order to identify tubule cells, endothelial cells, and podocytes.

### Results

Six murine kidney samples were evaluated by RNAscope™ 25 LS Multiplex Fluorescent ISH (two left and two right kidney samples from the treated mouse and one left and one right kidney sample from the naïve mouse). Mm-Ppib, Mm-Polr2a, Mm-Ubc, and Mm-Hprt were used as positive control markers for sample quality control and to evaluate RNA quality in the tissue samples. Bacterial gene dapB was used as a negative control. Optimization was performed to establish the best signal-to-noise ration.

RNAscope™ 25 LS Multiplex Fluorescent ISH was performed to evaluate the expression of EGFP-04 mRNA along with the expression of NPHS2, SCNN1A, and PECAM1 mRNAs in the six kidney samples. EGFP-04 expression indicated the expression of the genetic payload in the kidney cells. SCNN1A-positive cells indicated a tubular epithelial cell type. NPHS2-positive cells indicated a podocyte cell type. PECAM1 expression indicated an endothelial cell type. An example histological section is provided in FIG. 27. EGFP-04 mRNA expression was observed in all treated kidney samples and no detection was observed in the naïve sample. NPHS2, SCNN1A and PECAM1 mRNA expression were also observed throughout the kidney samples. EGFP-04 mRNA expression was observed in all treated samples and absent in the naïve sample. The various cell markers were also observed in all samples.

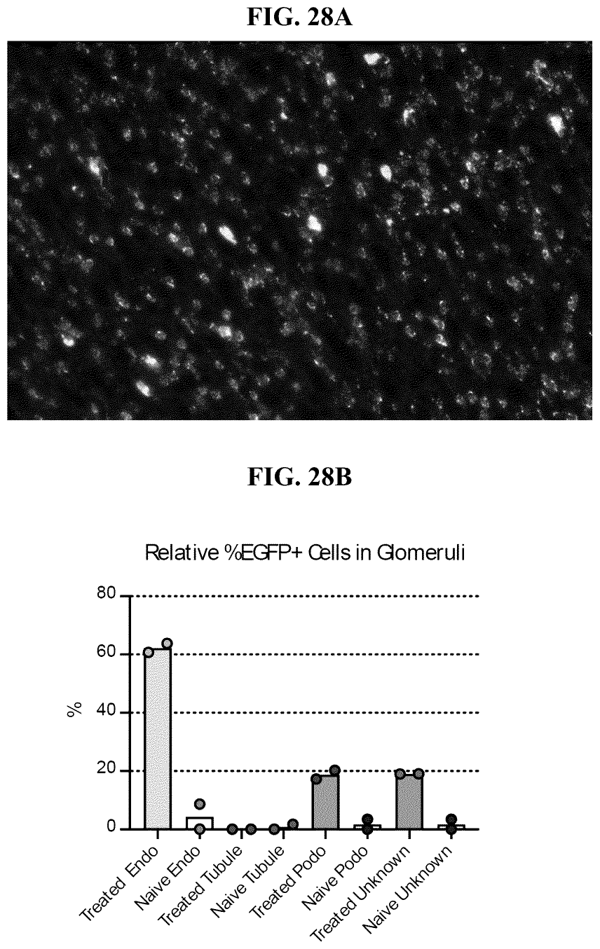

FIG. 28A provides an example histological sample with staining for EGFP, NPHS2, PECAM1, and SCNN1A in a non-glomerular area of the mouse kidney. FIG. 28B provides a quantitation of the percent of endothelial (PECAM1-positive), Tubular epithelial (SCNN1A-positive), and podocyte (NPHS2-positive) cells that were also EGFP-04 positive in treated and naïve samples in the non-glomerular areas. EGFP-04 expression was minimal in all three cell types in the naïve animal, while EGFP-04 expression was observed in endothelial and tubule cells in the treated animals, with the most expression seen in endothelial cells in the non-glomeruli area.

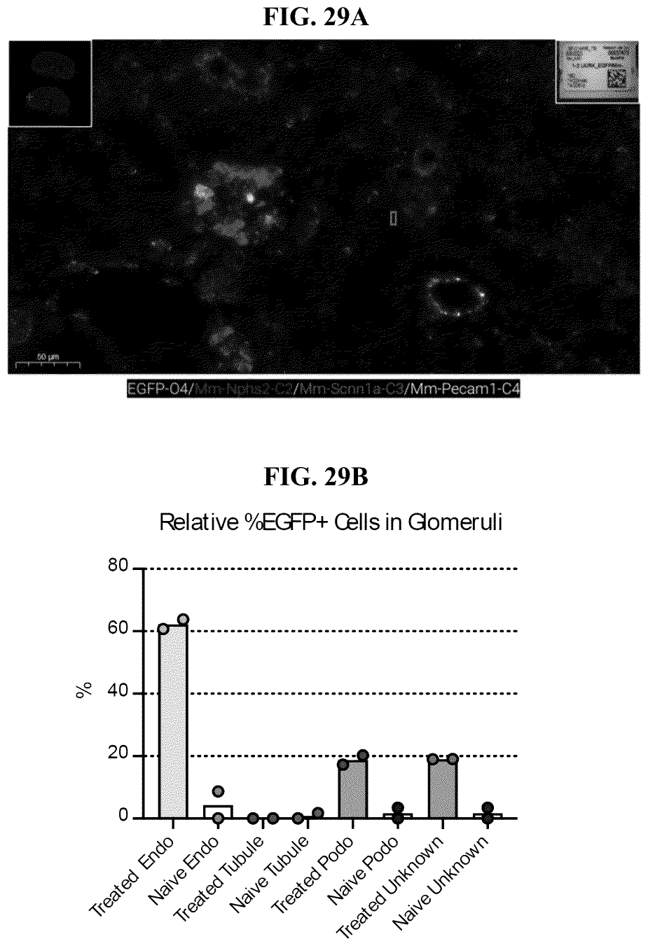

FIG. 29A provides an example histological sample with staining for EGFP, NPHS2, PECAM1, and SCNN1A in a glomerular area of the mouse kidney. FIG. 29B provides a quantitation of the percent of endothelial (PECAM1-positive), Tubular epithelial (SCNN1A-positive), podocyte (NPHS2-positive), and unknown cells that were also EGFP-04 positive in treated and naïve samples in the glomeruli area. EGFP-04 expression was minimal in all three cell types in the naïve animal, while EGFP-04 expression was seen in endothelial cells and podocytes in the treated animals, with the most expression seen in endothelial cells in the glomerular areas.

These results indicate that UMGD is effective in delivering a genetic payload to the murine kidney in endothelial, tubular epithelial, and podocyte cells in glomeruli and non-glomeruli areas.

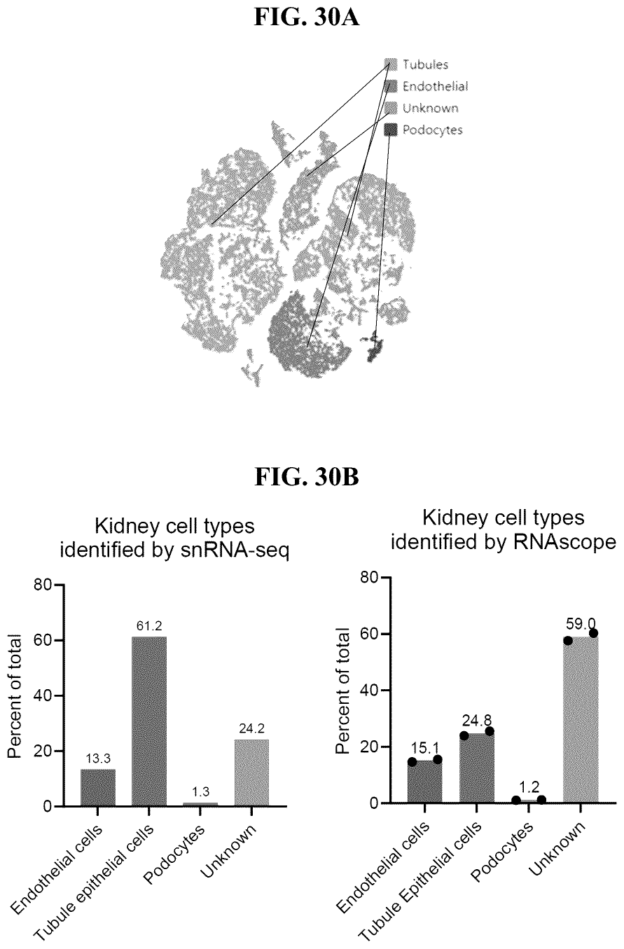

The distribution of endothelial cells, tubular epithelial cells and podocytes in the murine kidney was assayed using snRNA-seq. The spatial distribution of endothelial cells, tubular epithelial cells and podocytes in the murine kidney as detected by snRNAseq is provided in FIG. 30A. FIG. 30B provides a comparison of the percent of the total number of cells that are endothelial cells, tubular epithelial cells, podocytes, or unknown cells in the murine kidney as determined using snRNAseq (left) and RNAscope (right). While there are more unknown cells in RNAscope than in snRNAseq, both methods show the highest percentage of tubule endothelial cells, followed by endothelial cells, and then the lowest percentage of podocytes.

These results indicate that the repeated sonoporation with multiple organ location protocol effectively delivers and drives expression of a genetic payload in the murine kidney across multiple cell types.

### Example 15: Sonoporation in a Murine Liver Model Utilizing a Multiple Bolus and Multiple Ultrasound Location Protocol

### Animals and Protocol

There were two groups of two experimental animals, each of which was a C57 mouse. One experimental group was administered a nanoplasmid genetic payload and sonoactive microstructure mixture in conjunction with ultrasound (US) energy. The second experimental group was naïve and did not receive any intravenous injections of microbubbles or plasmids and did not receive externally applied ultrasound at any time.

Prior to the experiment, the treated mouse was implanted with a jugular vein catheter (JVC), through which the sonoactive microstructures and nucleic acid constructs were administered.

A dose of sonoactive microstructure and DNA solution was readied by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 225 μL of Optison into a syringe (dead space of the needle (about 50 microliters (uL) included in the calculations). With the same needle and syringe, 75 μL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into a patent JVC.

Each treatment session included three doses of mpDNA (Nanoplasmid) and sonoactive microstructure mixture in PBS administered as bolus injections, each delivered in through the jugular vein catheter. Each dose of mpDNA was about 83 μg and in total about 250 μg of mpDNA was delivered. The miniplasmid DNA construct comprised nucleic acids encoding a luciferase gene coupled to a CAG promoter (Np-CAG-Fluc).

Mice were administered 3 doses with one bolus per dose. For each dose, following administration of the microbubbles and nucleic acid payload, ultrasound acoustic energy was delivered to the liver area of mice in these experiments using a L6-24 probe using ultrasound imaging at the low mechanical index (MI) value of 0.09. Ultrasound was delivered continuously and alternated between a low mechanical index (MI) value of 0.07 and a high MI value of 1.5, without ceasing application of the ultrasound energy at any point during the treatment session. Nine flashes of high MI ultrasound at 1.5 were delivered with an interval of 4 seconds between each flash, and the administration of the 9 pulses was repeated three times. The high MI pulse duration was about 0.82 microseconds. The administration of the ultrasound per bolus injection was less than 30 seconds.

At the end of the experiment, the liver of the treated and untreated mice were sectioned. Two sections from the liver of the treated animal and two sections from the kidney of the naïve animal were analyzed using RNAscope to assess transfection of specific liver cell types using ultrasound-mediated gene delivery (UMGD).

Liver samples were analyzed using an RNAscope™ assay (RNAscope™ 25 LS Multiplex Fluorescent ISH Reagent Kit; Cat. No. 322800; and RNAscope™ LS 4-Plex Ancillary Kit for Multiplex Fluorescent Fluorescent ISH Reagent Kit; Cat. No. 322830). Based on assessment of tissue samples with reference positive and negative control probes, optimal pretreatment conditions for samples were established to maximize signal-to-noise ratio. Standard RNAscope™ Multiplex FL Assay pretreatment conditions were used as follows: (i) epitope retrieval 2:15 minutes at 95° C. and Protease III: 15 minutes at 40° C.

Positive and negative control probes are provided below.

| Control Probe Info | Probe Name | Probe Symbol | Catalog Number |
|---|---|---|---|
| ACD Positive Control Probe | Mus musculus Peptidylprolyl Isomerase B | Mm-Ppib-C1 | 313918-C2 |
| | (Cyclophilin B) | | |
| ACD Positive Control Probe | Mus musculus Polymerase (RNA) II | Mm-Polr2a-C2 | 312478-C3 |
| | (DNA directed) polypeptide A (POLR2A) | | |
| ACD Positive Control Probe | Mus musculus Ubiquitin C (UBC) | Mm-Ubc-C3 | 310778-C3 |
| ACD Positive Control Probe | Mus musculus Hypoxanthine | Mm-Hprt-C4 | 312958-C4 |
| | Phosphoribosyltransferase | | |
| ACD Negative Control Probe | Bascillus subtilis gene dihydrodipicolinate | dapB-C1/dapB-C2/ | 312038 |
| | reductase | dapB-C3/dapB-C4 | |

Target probes were: (1) Enhanced green fluorescent protein (EGFP), mRNA; EGFP-04; Cat. No. 538858; GenBack Accession Number MN623123.1; (2) Mus musculus asialoglycoprotein receptor 1 (ASGR1), mRNA; Mm-Asgr1-C2; Cat. No. 313738-C2; GenBack Accession Number NM_009714.2; (3) Mus musculus melanoma cell adhesion molecule 1 (MCAM1), mRNA; Mm-MCAM-C3; Cat. No. 406328-C3; GenBack Accession Number NM_023061.2; and (4) Mus musculus adhesion G protein-coupled receptor E1 (ADGRE1), mRNA; Mm-Adgre1-C4; Cat. No. 460658-C4; GenBack Accession Number NM_010130.4.

The target probes indicated: (1) EGFP: payload expression in liver cells; (2) NPHS2: podocytes; (3) SCNN1a: tubular epithelial cells; and (4) PECAM1: endothelial cells (FIG. 27). These probes are summarized below.

| Catalog | | | Probe GenBank | # of ZZ | Gene Region Probes |
|---|---|---|---|---|---|
| Number | Probe Name | Probe Symbol | Accession Number | Probe Pairs | Designed Against |
| 538858 | Enhanced green fluorescent protein | EGFP-O4 | MN623123.1 | 18 | 1166-1885 nt |
| | (EGFP), mRNA | | | | |
| 313738-C2 | Mus musculus asialoglycoprotein receptor | Mm-Asgr1-C2 | NM_009714.2 | 20 | 162-1174 nt |
| | 1 (ASGR1), mRNA | | | | |
| 406328-C3 | Mus musculus melanoma cell adhesion | Mm-Mcam-C3 | NM_023061.2 | 20 | 200-1119 nt |
| | molecule (MCAM), mRNA | | | | |
| 460658-C4 | Mus musculus adhesion G protein-coupled | Mm-Adgre1-C4 | NM_010130.4 | 20 | 85-1026 nt |
| | receptor E1 (ADGRE1), mRNA | | | | |

Visual scoring was performed to assign a single score to a sample based on the predominant staining pattern throughout the entire sample. Intensity of the stain did not have an impact on scoring. Dots in histological images correlate to the number of individual RNA molecule. Percentage of cells positive was scored visual based on the number of cells with more than 1 dot per cell and binned into categories (i.e., 0%, 1-25%, 26-25%, 51-75%, 76-99%, 100%).

Single nucleus RNA sequencing (snRNAseq) was also performed on the liver samples to profile gene expression in the liver cells in order to identify tubule cells, endothelial cells, and podocytes.

### Results

Four murine liver samples were evaluated by RNAscope™ 25 LS Multiplex Fluorescent ISH (two liver samples from the treated mouse and two liver samples from the naïve mouse). Mm-Ppib, Mm-Polr2a, Mm-Ubc, and Mm-Hprt were used as positive control markers for sample quality control and to evaluate RNA quality in the tissue samples. Bacterial gene dapB was used as a negative control. Optimization was performed to establish the best signal-to-noise ration.

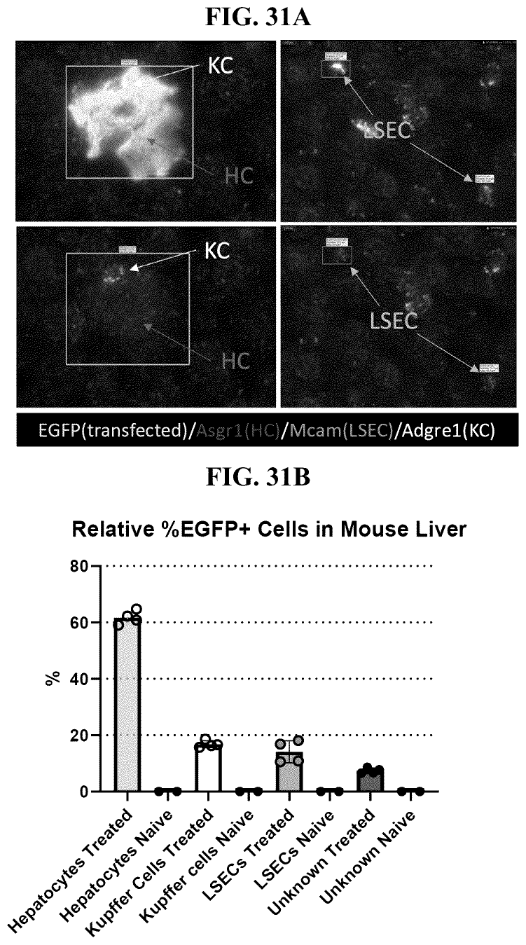

RNAscope™ 25 LS Multiplex Fluorescent ISH was performed to evaluate the expression of EGFP-04 mRNA along with the expression of MCAM, ADGRE1, and ASGR1 mRNAs in the four liver samples. EGFP-04 expression indicated the expression of the genetic payload in the liver cells. MCAM-positive cells indicated a liver sinusoidal endothelial cell (LSEC) type. Asgr1-positive cells indicated a hepatocyte cell (HC) type. Adgre1 expression indicated a Kupffer cell (KC) type. An example histological section is provided in FIG. 31A. EGFP-04 mRNA expression was observed in all treated liver samples and no detection was observed in the naïve sample. MCAM, ADGRE1, and ASGR1 mRNA expression were also observed throughout the liver samples, both treated and naïve. EGFP-04 mRNA expression was observed in all treated samples and absent in the naïve sample. The various cell markers were also observed in all samples.

FIG. 31A provides an example histological sample with staining for MCAM, ADGRE1, and ASGR1 in the mouse liver. FIG. 31B provides a quantitation of the percent of LSEC (MCAM-positive), hepatocyte (Asgr1-positive), and Kupffer (NPHS2-positive) cells that were also EGFP-04 positive in treated and naïve liver samples. EGFP-04 expression was minimal in all three cell types in the naïve animal, while EGFP-04 expression was observed in all three cell types in the treated animals, with the most expression seen in hepatocytes. Approximately 60% of all relative EGFP+ cells in the murine liver were hepatocytes.

These results indicate that UMGD is effective in delivering a genetic payload to the murine liver in LSEC, hepatocyte and Kupffer cells.

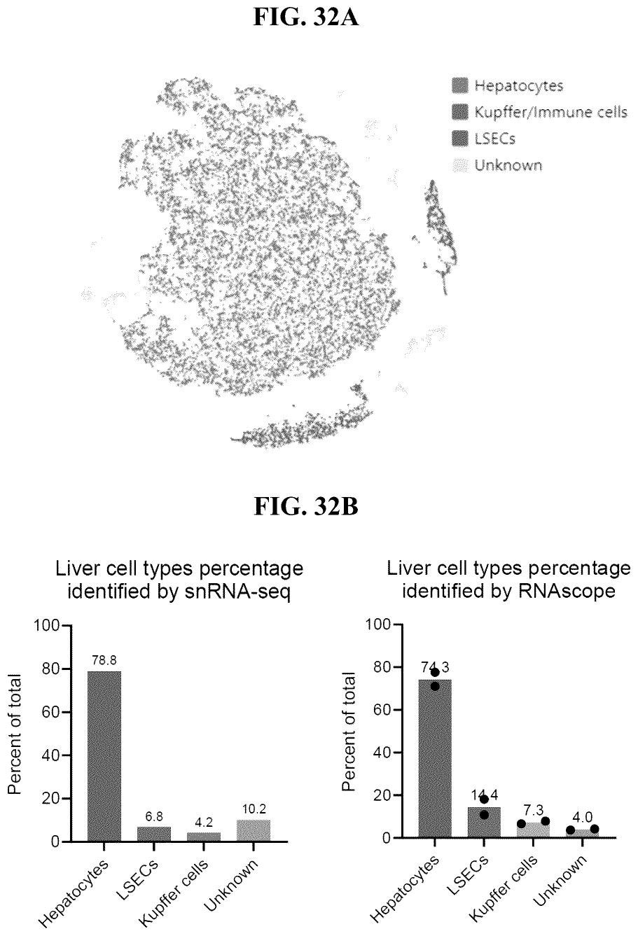

The distribution of LSEC, hepatocyte and Kupffer cells in the murine liver was assayed using snRNA-seq. The spatial distribution of LSEC, hepatocyte and Kupffer in the murine liver as detected by snRNAseq is provided in FIG. 32A. FIG. 32B provides a comparison of the percent of the total number of cells that are LSEC, hepatocyte and Kupffer, or unknown cells in the murine liver as determined using snRNAseq (left) and RNAscope (right). EGFP-04 expression was observed in all three cell types in the treated animals, with the most expression seen in hepatocytes. Approximately 78.8% of all relative EGFP+ cells in the murine liver were hepatocytes as measured by snRNA-seq. Approximately 74.3% of all relative EGFP+ cells in the murine liver were hepatocytes as measured by snRNA-seq. Both RNAscope and snRNAseq methods show the highest percentage of hepatocytes, followed by LSEC cells, and then the lowest percentage of Kupffer cells.

### Example 16: Expression of a Genetic Payload in NHP Kidney Cells

### Animals and Protocol

Following methods provided herein, nonhuman primates (NHPs) underwent ultrasound mediated gene delivery (UMGD) to deliver a genetic payload of enhanced green fluorescent protein (EGFP). Sections of the NHP kidney were analyzed using RNAscope to assess transfection of kidney cell using UMGD.

Kidney samples from (5) nonhuman primates (NHPs) were analyzed using an RNAscope™ assay (RNAscope™ 2.5 LSx Red Reagent Kit; Cat. No. 322750). Based on assessment of tissue samples with reference positive and negative control probes, optimal pretreatment conditions for samples were established to maximize signal-to-noise ratio. Standard RNAscope™ 2.5 LSx Red Assay pretreatment conditions were used as follows: (i) epitope retrieval 2:15 minutes at 95° C. Protease III: 15 minutes at 40° C.

Control probes used were: (i) ACD positive control probe (Macaca fascicularis, peptidylprlul Isomerase B (Cyclophilin B); Mfa-PPIB; Cat. No. 424148); ACD negative control probe (Bacillus subtilis dihydrodipicolinate reductase; dapB; Cat. No. 312038). The target probe was (Mycobacterium tuberculosis H37Rv plasmid pTYGi9; EGFP-04; Cat. No. 538858; GenBack Accession Number MN623123.1).

Visual scoring was performed to assign a single score to a sample based on the predominant staining pattern throughout the entire sample. Intensity of the stain did not have an impact on scoring. Dots in histological images correlate to the number of individual RNA molecule. Percentage of cells positive was scored visual based on the number of cells with more than 1 dot per cell and binned into categories (i.e., 0%, 1-25%, 26-25%, 51-75%, 76-99%, 100%)

### Results

All sampled passed a quality control check with moderate PPIB positive control staining and little to no dapB background staining. RNAscope™ 2.5 LSx Red Assay was performed in all samples to evaluate EGFP expression in NHP kidney samples. EGFP was detected in the kidney samples. Examples of histological images of the samples are provided in FIG. 23A (arrows indicate EGFP positive cells). FIG. 23B provides the percent of glomeruli positive for EGFP in kidney sections of NHP. These results indicate that UMGD is effective in delivering a genetic payload to the NHP kidney.

### Example 17: DNA Transfection Via Sonoporation Using a Multiple Organ Location and Repeated Protocol a in Murine Model of Polycystic Kidney Disease

### Animals and Protocol

In this example, 4 experimental groups evaluating transfection of a Fluc Reporter gene to a murine model of polycystic kidney disease was performed. Subject mice were C57, wild type, or mice homozygous for the Nek^{8ick} mutation which induces development of polycystic kidney disease. Mice homozygous for the Nek^{8ick} mutation exhibited advanced cystic pathology. Subjects were implanted with a jugular vein catheter prior to undergoing a sonoporation treatment for delivery of a Firefly luciferase to the kidneys. Subjects received a jugular vein catheter with infusion of nucleic acids encoding Firefly luciferase coupled to a CAG promoter, and either saline or protein stabilized sonoactive microstructures depending on the experimental group. Protein stabilized sonoactive microstructures utilized were Optison protein stabilized microspheres, and were prepared according to the instructions on the label as described in previous examples herein. Each subject received 3 doses of the nucleic acid and microbubble solution administered as a bolus injection followed by three applications of ultrasound acoustic energy to each kidney following administration of the bolus injection of the saline or sonoactive microstructures and nucleic acid. Dosing intervals between the repeat administrations of the nucleic acid and the saline or sonoactive microstructures with application of ultrasound acoustic energy was four hours. Ultrasound acoustic energy was applied to the distal cortex of the kidney, and alternated between cross sectional view and a longitudinal views. Ultrasound acoustic energy was applied at an alternating mechanical index alternating between a low MI value of 0.9 and a high MI value of 1.3, applied using 27 high MI flashes with A2 second intra flash time, using an L6-24 ultrasound probe. Approximately 150 microliters of saline or protein stabilized sonoactive microstructures, and approximately 50 microliters of saline comprising the nucleic acids were administered per dose, for a total infusion volume of 200 microliters. Subjects underwent IVIS analysis at 1, 2, 3, and 7 days post treatment, after which kidney tissue underwent ⅓ N BF fixation and drying prior to flash freezing and storage, and were subsequently evaluated using immunohistochemistry and qPCR readouts. The experimental conditions are summarized below.

| | | | | | | Intra | | Flashes | | | Optison | | |
|---|---|---|---|---|---|---|---|---|---|---|---|---|---|
| | | | | Dosing | | flash | Location | per | | Test Article | Dose | Sample | |
| Group | Doses | N | Strain | Intervals | Region | time | (bolus) | location | US | (Np-CAG- [symbol] ) | (IV) | Collection | Readouts |
| 1 | 3 | 5 | NEK8 | 4 Hour | Distal | 2 | 1 Bolus/ | | L6-24 | 3 x | N/A | D 8 Kidney | IHC |
| 2 | | 10 | NEK8 | | Corext | second | 27 F [symbol] hes | | Res | 116 u mol | 150 μL/ | (L [symbol] & R [symbol] ) | qPCR |
| 3-a | | 5 | “Wt” | | | | | | (0.9 MI, | (277 ug), | Dose | Liver, Heart, | |
| 3-b | | 3 | C57 | | | | | | 1.3 CM) | 348 u mol | | Lung | |
| | | | | | | | | | | total | | | |

[symbol]

indicates data missing or illegible when filed

Fixation and Drying

| 10% NBF | | |
|---|---|---|
| ↓ | 24 | H |
| 70% EtOH | | |
| ↓ | 30-60 | min |
| 80% EtOH | | |
| ↓ | 30-60 | min |
| 90% EtOH | | |
| ↓ | 30-60 | min |
| 95% EtOH | | |
| ↓ | 30-60 | min |
| 100% EtOH | | |
| ↓ | 30-60 | min |
| 100% EtOH | | |
| ↓ | 30-60 | min |

| | |
|---|---|
| 100% EtOH | → Storage |

### Results

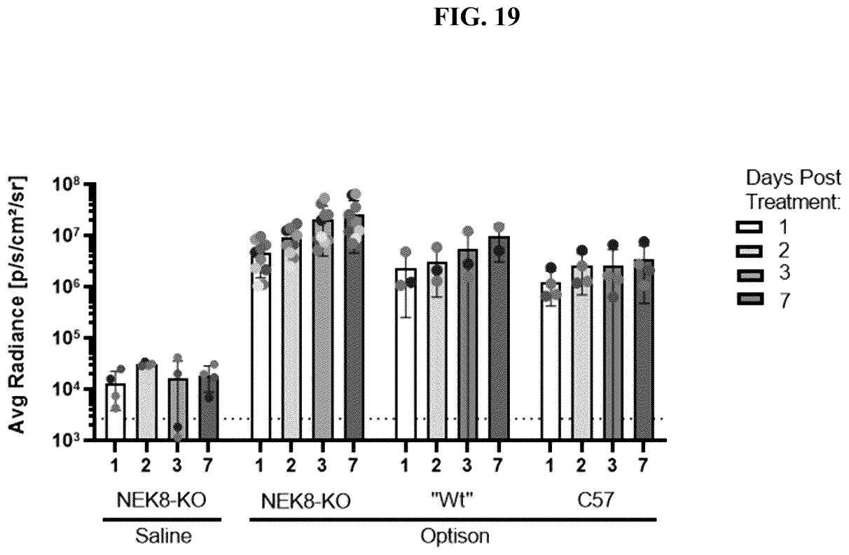

Using IVIS, mice were imaged 1, 2, 3, and 7, days after administration of the nucleic acids, microbubbles, and ultrasound energy. FIG. 19 shows the average radiance from the IVIS images from mice in the various experimental groups at the indicated time points. Average radiance measurements were in the range of 10\hat{ }6 to 10\hat{ }7 for the wild type and C57 groups, and exceeded 10\hat{ }7 for the Nek^{8ick} mutation. The negative control group administered only saline showed radiance signals of only around 10\hat{ }4 consistent with background noise.

As shown in FIG. 19, expression of luciferase is observed in all experimental groups 24 hours after administration and persists for at least 7 days. Sonoporation showed initial differences between the experimental groups. The control group administered no sonoactive microstructures exhibited low but detectable signal. Mice homozygous for the Nek^{8ick} mutation exhibiting bands to cystic pathology with increased kidney size exhibited increased levels of gene transfection and expression as compared to wild type or C57 mice.

| Group | Mean Radiance | SD | D 7 Max | N |
|---|---|---|---|---|
| NEK-8 + Saline | 1.9e4 | ±1.0e4 | 3.1e5 | 4 |
| NEK-8 + Optison | 2.7e7 | ±2.2e7 | 6.4e7 | 10 |
| Wt + Optison | 1.0e7 | ±6.9e6 | 1.5e7 | 2* |
| C57 + Optison | 3.4e6 | ±2.9e6 | 7.7e6 | 4 |

Immumnofluorescence for kidney luciferase expression from frozen tissues was conducted. The frozen kidney tissues were obtained and slides prepared. The slides were stored at −80 C before staining. After staining the slides were stored at 4 C. The protocol for staining is as below:

### Frozen Section Protocol

[symbol] Allow frozen kidney section slides to come to room temperature (˜30 min)
[symbol] Rinse in PBS for 5 min at RT
[symbol] Fix in 4% paraformaldehyde for 5 min at RT
[symbol] Wash in PBS for 5 min (2×) at RT
[symbol] Wash in PBT (0.25% TritonX-100) for 5 min at RT
[symbol] Wash in PBS for 5 mins at RT
[symbol] Block in BlockAid blocking solution for 1 h at RT
[symbol] Primary Abs in BlockAid blocking solution (humidified chamber) O/N at 4° C.
[symbol] Next morning wash in PBS (×5) for 3 min each at RT
[symbol] Secondary Abs in BlockAid blocking solution in PBS for 2 h (protect from light) at RT
[symbol] Wash in PBS (×5) for 3 min each at RT (protect from light)
[symbol] Mount with Vector H-1200) with DAPI
[symbol] Store slides at 40C

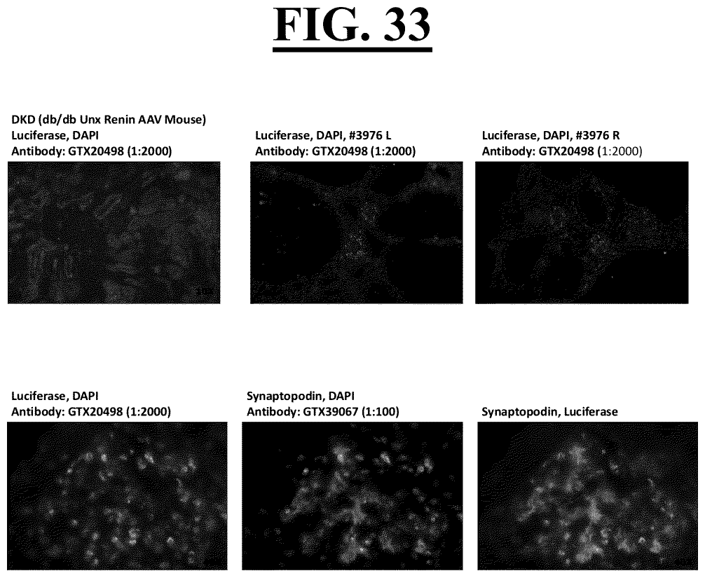

The slides were visualized with the Nikon Eclipse E600 microscope and images were captured using the VisiView (Version 4.5.0.0, license #1646) software. As expected, staining with GTX antibody GTX20498 did not produce any signals in the DKD mice without any gene delivery by sonoporation. Luciferase (Green) signals were obtained in glomerulus of the kidneys with gene delivery by sonoporation. The immunofluorescence staining method for luciferase was optimized for frozen kidney sections. Luciferase antibody that did not produce any background signal in frozen kidneys (from prior internal studies) was identified Luciferase signal was detected in glomerulus, confirmed by synaptopodin IF staining. The results of the IF staining are shown in FIG. 33.

### Example 18: Long Term Dose Response from Multiple Payload Doses and Multiple Ultrasound Administrations

### Experimental Animals and Protocol

Four groups of four BALB/c mice were administered sonoactive microstructures and nanoplasmid encoding a luciferase, delivered intravenously through a jugular vein catheter. The first group received administration of 5 micrograms of nucleic acids, the second group received administration of 50 micrograms of nucleic acids, the third experimental group received administration of 100 micrograms of nucleic acid, and the fourth group received administration and 250 micrograms of nucleic acid; each suspended in 50 microliters of phosphate buffered saline.

Mice were administered three doses as a bolus injection of nanoplasmid format nucleic acids and sonoactive microstructures (“microbubbles). Each group was administered their respective dosage of nucleic acid in 50 microliters of PBS, and 200 microliters of sonoactive microstructure suspensions, the nucleic acids and sonoactive microstructure defining a dose, over a period of 15 seconds. After administration of the nucleic acids and sonoactive microstructures, continuous ultrasound energy was administered transcutaneously to the liver at a first location alternating between a low MI and a high MI. Ultrasound was applied at a low mechanical index of 0.07, and a high mechanical index was applied in 9 flashes 4 seconds apart at a high MI of 0.8. Following application of the ultrasound energy, the sonoactive microstructure and nucleic acids were re-administrated and ultrasound was reapplied. The three doses of the nanoplasmid, microbubbles, and ultrasound energy were completed during one treatment session. Luciferase expression was measured by IVIS at 1 day 2 days, 1, 2, 3, 4, 5, 6, 7, 8, 9, 10, 11, 12, 14, 16, 18, 20, 22, 24, and 26 weeks following the treatment.

### Results

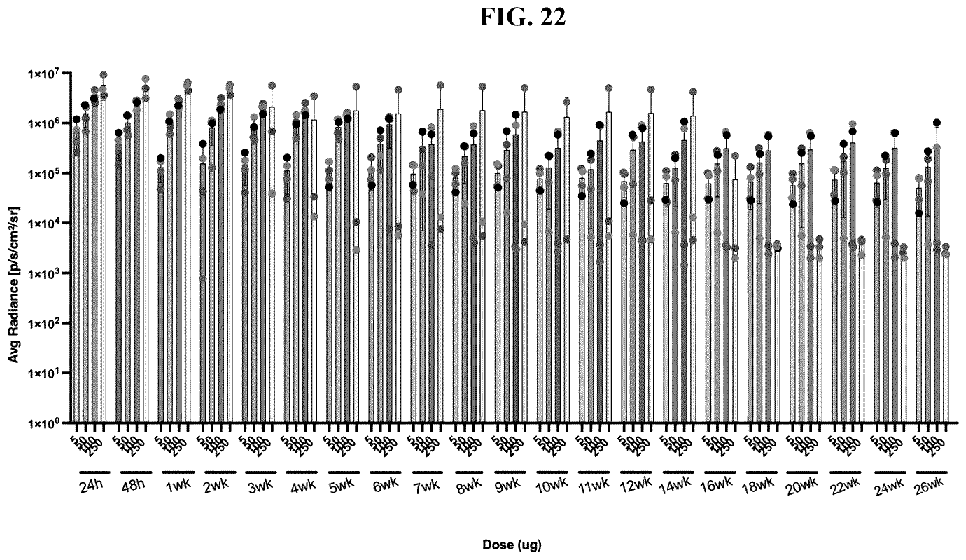

Using IVIS, mice were imaged at 1 day 2 days, 1, 2, 3, 4, 5, 6, 7, 8, 9, 10, 11, 12, 14, 16, 18, 20, 22, 24, and 26 weeks following the treatment. FIG. 22 shows the average IVIS radiance values from mice receiving the treatments, with IVIS analysis conducted at the indicated time points.

As shown in FIG. 22, expression of luciferase is observed in experimental groups at 24 hours after administration and persists for at least 16 weeks, and for the 5, 50, and 500 ug groups through 26 weeks. Throughout the duration of the study, expression levels appear to correlate with increased dosages of nucleic acids administered, with highest average expression levels observed in the experimental group receiving 250 micrograms of nucleic acids up to 14 weeks, and lowest expression levels in mice receiving 5 micrograms of nucleic acids (FIG. 22).

### Example 19: Long Term Durability in Murine Liver Model Utilizing Multiple Bolus and Multiple Ultrasound Location Administration

### Experimental Animals and Protocol

There were 2 experimental groups, each of which included four RAG2 knock-out mice. Prior to the experiment, each mouse was implanted with a jugular vein catheter (JVC) through which the sonoactive microstructure and nucleic acid constructs were administered.

Mice in this experiment received a single sonoporation session which included three boluses of miniplasmid DNA (mpDNA) and sonoactive microstructures administered in the treatment session. The mpDNA construct used in this experiment was a Nanoplasmid™ construct. Ultrasound (US) energy was delivered transcutaneously to transfect the target organ, the liver. Group 1 received three bolus injections of 33 μg of DNA for a total delivery of 100 μg of DNA payload administered in the treatment session, and Group 2 received three bolus injections of 76 μg of DNA payload for a total delivery of 228 μg of DNA administered in the treatment session. The DNA payload solution to sonoactive microstructure solution volumetric ratio utilized was 1:4.

A dose of sonoactive microstructure and DNA solution was prepared by first preparing the sonoactive microstructures as instructed on the label: microstructures were removed from 4C storage and rolled between the palms for 20 seconds; the protective plastic and aluminum covering from Optison vial was removed; a 25G needle was inserted through the rubber gasket of the Optison vial to provide a pressure vent; and a 1.5 inch 18G needle was used to draw up 12 mL of Optison into a syringe (dead space of the needle included in the calculations). With the same needle and syringe, either 228 μg or 100 μg of DNA payload was drawn into the syringe to combine the DNA and Optison. The Optison microbubbles and DNA payload were mixed in the syringe by rolling the syringe between the fingers until the solution was homogenous. The DNA+Optison solution was drawn out of the needle dead space. Then the 18G needle was exchanged for a 25G blunt needle for injection into the IV catheter.

Each session included either three bolus injections of 33 μg of DNA for a total delivery of 100 ug, or three bolus injections of 76 μg of DNA payload for a total delivery of 228 μg of DNA, and sonoactive microstructure mixture in PBS, each delivered through the jugular vein catheter. The miniplasmid DNA construct comprised nucleic acids encoding a luciferase gene coupled to an ApoE promoter. Sonoactive microstructures, Optison™ microbubbles, were delivered to the mice at a ratio of 1:4 (mpDNA solution: sonoactive microstructure mixture, including needle dead space dispersed into mixture), by volume.

Following administration of the first bolus of microbubbles and nucleic acid payload, ultrasound acoustic energy was delivered to the liver area of mice in these experiments using a L6-24 probe positioned perpendicular to the mouse to locate the lateral view of liver using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 0.8. Nine flashes of high MI ultrasound at 0.8 were delivered with an interval of 4 seconds between each flash, and the administration of the 9 pulses was repeated three times. The high MI pulse duration was about 0.82 microseconds. Upon administration of the second bolus of microbubbles and nucleic acid payload, the ultrasound probe was moved to a second location on the liver and ultrasound acoustic energy was delivered to a second liver location. Upon administration of the third bolus of microbubbles and nucleic acid payload, the ultrasound probe was moved to a third location on the liver and ultrasound acoustic energy was delivered to a third liver location.

### Results

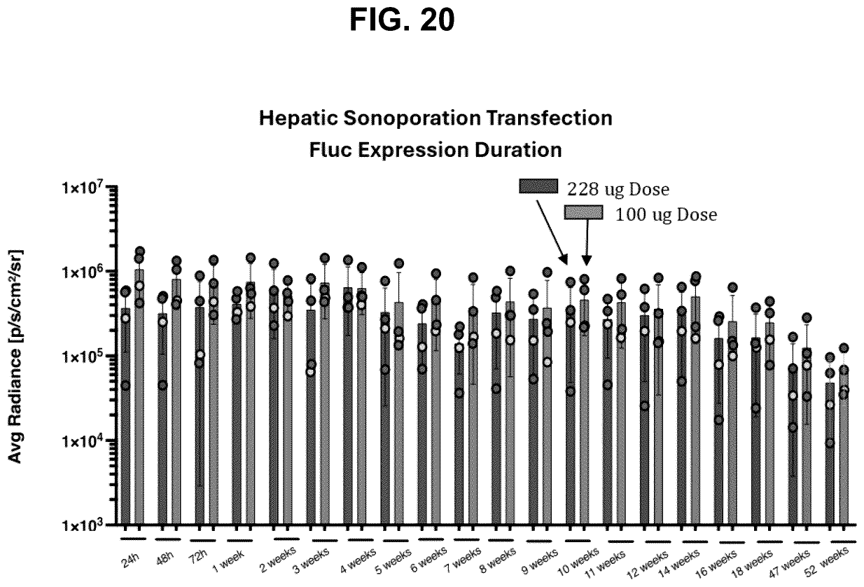

After the first session, IVIS fluorescence radiance imaging of all groups was performed at 24, 48, and 72 hours, and then repeated weekly for 52 weeks (FIG. 20). IVIS fluorescence imaging sessions were performed for all mice in each group at each time point. Large dots along the bar graphs represent individual mice readouts, and the bar graphs are the group averages. The left bars are the 228 ug group, and the right bars are the 100 ug group. It is observed that the mice exhibit stable levels of gene expression through 52 weeks when treated with a multiple bolus and multiple ultrasound location protocol.

### Example 20: Long Term Durability in Murine Liver Model Utilizing Multiple Bolus and Multiple Ultrasound Location Administration

### Experimental Animals and Protocol

One experimental group of 4 BALB/c mice was studied. The mice were 4 administered multiple boluses of DNA payload and sonoactive microstructures, and received administration of ultrasound applied to multiple organ locations, and were followed for 52 weeks to assess the durability gene expression. Prior to the experiment, each mouse was implanted with a jugular vein catheter (JVC), through which the sonoactive microstructures and nucleic acid constructs were administered.

A dose of sonoactive microstructure and DNA solution was readied by first preparing the sonoactive microstructures as instructed on the label: remove from 4C storage and roll between the palms for 20 seconds; removing protective plastic and aluminum covering from Optison vial; placing 25G needle through the rubber gasket to provide a pressure vent; and using 1.5 inch 18G needle to draw up 225 μL of Optison into a syringe (dead space of the needle (about 50 microliters (uL) included in the calculations). With the same needle and syringe, 75 μL of DNA payload was drawn into the syringe to combine the DNA and Optison. The Optison microbubbles and DNA payload were mixed in the syringe by rolling the syringe between the fingers until the solution is homogenous. The DNA+Optison solution was drawn out of the needle dead space. Then the 18G needle was exchanged for a 25G blunt needle for injection into a patent JVC.

Each treatment session included one dose of mpDNA and sonoactive microstructure mixture in PBS, totaling 150 uL, each delivered in through the jugular vein catheter. The miniplasmid DNA construct comprised nucleic acids encoding a luciferase gene coupled to a CAG promoter. Sonoactive microstructures, Optison™ microbubbles, were delivered to the mice at a ratio of 1:4 (mpDNA solution: sonoactive microstructure mixture, including needle dead space dispersed into mixture), by volume.

Mice were administered three bolus injections of about 33 μg of DNA payload and sonoactive microstructures totaling about 100 μg of DNA per treatment session. Following administration of the microbubbles and nucleic acid payload, ultrasound acoustic energy was delivered to the kidney area of mice in these experiments using a L6-24 probe positioned perpendicular to the mouse to locate the lateral view of kidney using B-mode ultrasound imaging at the low mechanical index (MI) value of 0.07. The depth setting was set to 2 cm, and the zoom to 0. Ultrasound was delivered continuously and alternated between a low mechanical index (MI) value of 0.07 and a high MI value of 0.8, without ceasing application of the ultrasound energy at any point during the treatment session. Nine flashes of high MI ultrasound at 0.8 were delivered with an interval of 4 seconds between each flash, and the administration of the 9 pulses was repeated three times. The high MI pulse duration was about 0.82 microseconds. Subsequent bolus injections of sonoactive microstructure and DNA solution were administered approximately every 30 seconds. Upon administration of the second bolus injection, the ultrasound prove was moved from a cross sectional view to a longitudinal views. Upon administration of the second bolus injection, the ultrasound prove was moved from a lateral sectional view back to a cross sectional view.

### Results

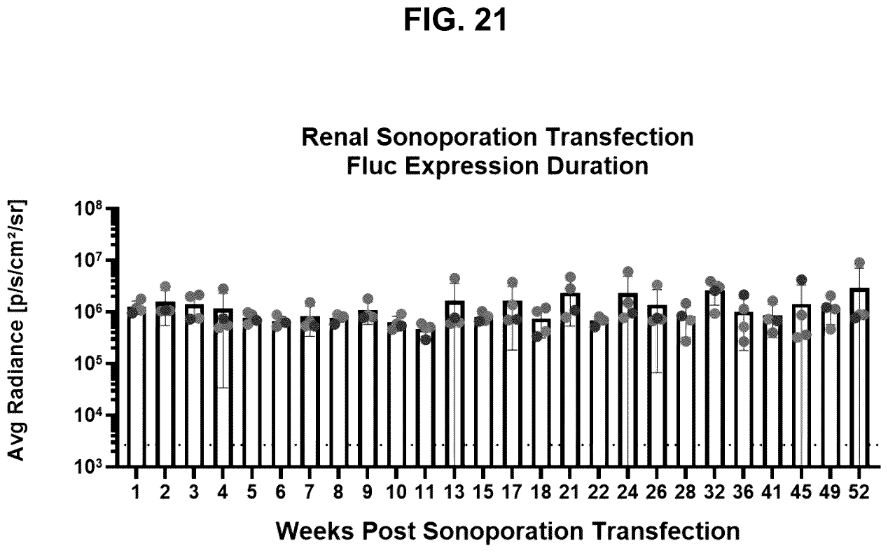

Using IVIS fluorescence imaging, mice were imaged at 1 week intervals, for 52 weeks. IVIS fluorescence radiance measurements of all groups indicated stable expression of the luciferase genetic payload in the murine kidney for 52 weeks (FIG. 21).

### Numbered Embodiments

1. A method of delivering a nucleic acid payload to a target cell of a subject comprising:
providing a treatment session to the subject, wherein the treatment session comprises:
a. administering the nucleic acid payload to the subject;
b. administering a plurality of microbubbles to the subject; and
c. administering ultrasound energy to the subject in proximity to the target cell(s); and
repeating the treatment session at least once more than 6 hours after but within 10 days after initiation of a first treatment session.

2. A method of delivering a nucleic acid payload to a target cell of a subject comprising:
providing a treatment session to the subject, wherein the treatment session comprises:
a. administering the nucleic acid payload to the subject;
b. administering a plurality of microbubbles to the subject; and
c. administering ultrasound energy to the subject in proximity to the target cell(s);
and repeating the treatment session at least once more than 21 days after initiation of a first treatment session.

3. A method of delivering a nucleic acid payload to a target cell of a subject comprising:
providing a treatment session to the subject, wherein the treatment session comprises:
a. administering the nucleic acid payload to the subject;
b. administering a plurality of microbubbles to the subject; and
c. administering ultrasound energy to the subject in proximity to the target cell(s); and
repeating the treatment session,
wherein the repeating the treatment session increases expression of a protein encoded by the nucleic acid payload by at least 5%, 10%, 20%, 30%, 40%, 50%, 100%, 250%, 500%, 750%, 1000%, or 2000% as compared to single dose administration.

4. A method of delivering a nucleic acid payload to a target cell of a subject comprising:
providing a treatment session to the subject, wherein the treatment session comprises:
administering two or more doses the nucleic acid payload to the subject;
administering two or more doses of a plurality of microbubbles to the subject; and
administering ultrasound energy to the subject in proximity to the target cell(s); and
repeating the treatment session.

5. The method of any one of the preceding embodiments, wherein the two or more doses the nucleic acid payload comprises three or more doses.

6. The method of any one of the preceding embodiments, wherein the two or more doses of the nucleic acid payload comprise three or more doses of a plurality of microbubbles.

7. The method of any one of the preceding embodiments, wherein the repeating the treatment session increases expression of a protein encoded by the nucleic acid payload by at least 5%, 10%, 20%, 30%, 40%, 50%, 100%, 250%, 500%, 750%, 1000%, or 2000% as compared to single dose administration.

8. The method of any one of the preceding embodiments, wherein the repeating the treatment session comprises reapplying the ultrasound energy to a second location on a target organ in proximity to the target cell(s).

9. The method of any one of the preceding embodiments, wherein the repeating the treatment session comprises reapplying the ultrasound energy to a second or subsequent location on a target organ in proximity to the target cell(s).

10. The method of any one of the preceding embodiments, wherein the repeating the treatment session comprises reapplying the ultrasound energy to a same location on a target organ in proximity to the target cell(s).

11. The method of any one of the preceding embodiments, wherein the administering the nucleic acid payload to the subject comprises administering two or more doses of the nucleic acid payload to the subject during the treatment session.

12. The method of any one of the preceding embodiments, wherein the administering the nucleic acid payload to the subject comprises administering three or more doses of the nucleic acid payload to the subject during the treatment session.

13. The method of any one of the preceding embodiments, wherein the administering the plurality of microbubbles to the subject to the subject comprises administering two or more doses of the plurality of microbubbles to the subject during the treatment session.

14. The method of any one of the preceding embodiments, wherein the administering the plurality of microbubbles to the subject to the subject comprises administering three or more doses of the plurality of microbubbles to the subject during the treatment session.

15. The method of any one of the preceding embodiments, wherein the repeating the treatment session comprises re-administering two or more doses of the nucleic acid payload to the subject during the treatment session.

16. The method of any one of the preceding embodiments, wherein the repeating the treatment session comprises re-administering three or more doses of the nucleic acid payload to the subject during the treatment session.

17. The method of any one of the preceding embodiments, wherein the repeating the treatment session comprises re-administering two or more doses of the plurality of microbubbles to the subject during the treatment session.

18. The method of any one of the preceding embodiments, wherein the repeating the treatment session comprises re-administering three or more doses of the plurality of microbubbles to the subject during the treatment session.

19. The method of any one of the preceding embodiments, wherein the nucleic acid payload is a nanoplasmid.

20. The method of any one of the preceding embodiments, wherein the nucleic acid payload comprises a therapeutic transgene coupled to a promoter other than CMV promoter or ubiquitin C promoter.

21. The method of any one of the preceding embodiments, wherein the ultrasound energy is sufficient to disrupt the microbubbles.

22 The method of any one of the preceding embodiments, wherein the ultrasound energy is sufficient to allow the nucleic acid payload to enter the target cell.

23. The method of any one of the preceding embodiments, wherein the ultrasound energy is sufficient to allow the nucleic acid payload to enter the target cell, as demonstrated by expression of the nucleic acid or amino acid sequence.

24. The method of any one of the preceding embodiments, wherein the ultrasound energy comprises applying ultrasound energy at a mechanical index ranging from 0.05 to 2.3.

25. The method of any one of the preceding embodiments, wherein the ultrasound energy comprises applying ultrasound energy at a mechanical index ranging from 0.05 to 2.1.

26. The method of any one of the preceding embodiments, wherein the ultrasound energy comprises applying ultrasound energy at a mechanical index ranging from 0.05 to 1.6.

27. The method of any one of the preceding embodiments, wherein ultrasound energy comprises applying ultrasound energy for up to 30, 60, 90, 120, 150, 180, 240, 300, or 360 seconds.

28. The method of any one of the preceding embodiments, wherein ultrasound energy comprises applying ultrasound energy for up to 10, 15, 20, 25, or 30 minutes.

29. The method of any one of the preceding embodiments, wherein the administering the ultrasound energy comprises continuously applying ultrasound energy for the duration of the treatment session.

30. The method of any one of the preceding embodiments, wherein continuously applying ultrasound energy comprises applying ultrasound energy at a first mechanical index for a first duration, applying ultrasound energy at a second mechanical index for a second duration, and re-applying ultrasound energy at the first mechanical index without ceasing application of ultrasound energy for the duration of the continuous application of ultrasound energy.

31. The method of any one of the preceding embodiments, second mechanical index is higher than the first mechanical index.

32. The method of any one of the preceding embodiments, wherein continuously applying ultrasound energy comprises applying ultrasound energy at a first mechanical index for a first duration, applying ultrasound energy at a second mechanical index for a second duration, and re-applying ultrasound energy at the first mechanical index without ceasing application of ultrasound energy for the duration of the treatment session.

33. The method of any one of the preceding embodiments, wherein the treatment session as determined by the length of continuous application of ultrasound energy is up to 30, 60, 90, 120, 150, 180, 240, 300, or 360 seconds.

34. The method of any one of the preceding embodiments, wherein the administering of the ultrasound energy of c. comprises administering a plurality of ultrasound flashes in which the mechanical index is increased, the plurality of ultrasound flashes administered at least 2 seconds apart from one another.

35. The method of any one of the preceding embodiments, wherein the administering of the ultrasound energy of c. comprises administering a plurality of ultrasound flashes in which the mechanical index is increased, with each of the plurality of ultrasound flashes is administered within 1 s to about 10 s apart from one another.

36. The method of any one of the preceding embodiments, wherein the administering of the ultrasound energy of c. comprises administering a plurality of ultrasound flashes in which the mechanical index is increased, with each of the plurality of ultrasound flashes administered within 5 s to about 10 s apart from one another over about 0.5 s.

37. The method of any one of the preceding embodiments, wherein the administering of the ultrasound energy of c. comprises administering a plurality of 10 ultrasound flashes in which the mechanical index is increased, with each of the plurality of 10 ultrasound flashes administered within 1 s to about 2 s apart from one over about 0.5 s.

38. The method of any one of the preceding embodiments, wherein the ultrasound flash is a change in the application of the ultrasound energy from the first mechanical index to the second mechanical index, and reapplication of the ultrasound energy at the first mechanical index from the second mechanical index within a time period.

39. The method of any one of the preceding embodiments, wherein the time period is less than 1 s.

40. The method of any one of the preceding embodiments, wherein the time period is from about 0.7 us to about 3 us (microseconds).

41. The method of any one of the preceding embodiments, wherein the time period is about 0.72, 0.82, 0.72, 0.98, or 2.28 us.

42. The method of any one of the preceding embodiments, wherein the administering of the ultrasound energy of c. comprises administering at least 5 ultrasound flashes at a second mechanical index which is increased relative to a first mechanical index less than 10 seconds apart from one another.

43. The method of any one of the preceding embodiments, wherein the administering the nucleic acid payload or administering the plurality of microbubbles comprises administering intravenously through a peripheral vein.

44. The method of any one of the preceding embodiments, wherein the expression of the nucleic acid or amino acid sequence encoded by the nucleic acid payload is sustained at a level of at least 50% of a peak expression level of the protein or the mRNA for at least 1 week.

45. The method of any one of the preceding embodiments, wherein the expression of the nucleic acid or amino acid sequence encoded by the nucleic acid payload is sustained at a level of at least 50% of a peak expression level of the protein or the mRNA for at least 1 week.

46. The method of any one of the preceding embodiments, wherein the treatment session is repeated within 72 hours after an initial performance of a first treatment session.

47. The method of any one of the preceding embodiments, wherein the treatment session is repeated within 48 hours after an initial performance of a first treatment session.

48. The method of any one of the preceding embodiments, wherein the treatment session is repeated twice within 24 hours after performance of a first treatment session.

49. The method of any one of the preceding embodiments, wherein the treatment session is repeated 30 days after performance of a first treatment session.

50. The method of any one of the preceding embodiments, wherein the treatment session is repeated 60 days after performance of a first treatment session.

51. The method of any one of the preceding embodiments, wherein the treatment session is repeated 90 days after performance of a first treatment session.

52. The method of any one of the preceding embodiments, wherein the treatment session is repeated at least 90 days after performance of a first treatment session.

53. The method of any one of the preceding embodiments, wherein the treatment session is repeated at least 100 days after performance of a first treatment session.

54. The method of any one of the preceding embodiments, wherein the treatment session is repeated at least 110 days after performance of a first treatment session.

55. The method of any one of the preceding embodiments, wherein the treatment session is repeated 120 days after performance of a first treatment session.

56. The method of any one of the preceding embodiments, wherein the treatment session is repeated at least 120 days after performance of a first treatment session.

57. The method of any one of the preceding embodiments, wherein repeating the treatment session does not result in a substantial elevation of inflammatory biomarker(s).

58. The method of any one of the preceding embodiments, wherein repeating the treatment results in elevation of inflammatory biomarker(s) within 20% of a baseline level of the inflammatory biomarker(s).

59. The method of the preceding embodiment, wherein the biomarker(s) markers are: ALT, AST, IL6, or combinations thereof.

60. The method of the preceding embodiment, wherein the nanoplasmid construct is administered at a concentration of 0.4 mg/kg body mass to 1.5 mg/kg body mass.

61. The method of any one of the preceding embodiments, wherein the administering the nucleic acid payload to the subject, the administering a plurality of microbubbles to the subject, and the administering ultrasound energy to the subject induces expression of the payload in the target cell within 24 hours.

62. The method of any one of the preceding embodiments, wherein the expression of the nucleic acid payload in the target cell is maintained for at least 1, 2, 3, 4, 5, 6, 7, 8, 9, 10, 11, 12, 13, 14, 15, or 16 weeks following performance of steps a.-c.

63. The method of any one of the preceding embodiments, wherein the expression of the nucleic acid payload in the target cell is maintained at an elevated level as compared to a baseline expression level for at least 1, 2, 3, 4, 5, 6, 7, 8, 9, 10, 11, 12, 13, 14, 15, or 16 weeks following performance of steps a.-c.

64. The method of any one of the preceding embodiments, wherein the elevated level of the nucleic acid payload as compared to a baseline expression level of the is at least 10% greater than the baseline expression level.

65. The method of any one of the preceding embodiments, wherein the administering of the nucleic acid payload of a. is administered at dosage of about 3.5 μg/μl.

66. The method of any one of the preceding embodiments, wherein the microbubbles comprise a protein stabilized shell.

67. The method of any one of the preceding embodiments, wherein the microbubbles comprise an albumin stabilized shell.

68. The method of any one of the preceding embodiments, wherein the microbubbles comprise perflutren gas.

69. The method of any one of the preceding embodiments, wherein the microbubble is an Optison microbubble.

70. The method of any one of the preceding embodiments, wherein the microbubbles comprise a lipid stabilized shell.

71. The method of any one of the preceding embodiments, wherein a concentration of the microbubbles administered is up to about 1.2*10\hat{ }10 microbubbles/ml.

72. The method of any one of the preceding embodiments, wherein the microbubbles are administered at a concentration of at least 5*10\hat{ }8 microbubbles/ml.

73. The method of any one of the preceding embodiments, wherein a concentration of the microbubbles administered is between 5*10\hat{ }8 and 8*10\hat{ }8 microbubbles/ml.

74. The method of the preceding embodiment, wherein the administering ultrasound energy occurs transcutaneously.

75. The method of any one of the preceding embodiments, wherein the target cell(s) comprise hepatic cell(s).

76. The method of any one of the preceding embodiments, wherein the target cell(s) comprise renal cell(s).

77. The method of any one of the preceding embodiments, wherein the target cell(s) comprise pancreatic cell(s).

78. The method of any one of the preceding embodiments, wherein the target cell(s) comprise cardiac cell(s), or endothelial cell(s).

79 The method of any one of the preceding embodiments, wherein the target cell(s) comprise myocyte cell(s), skeletal muscle cell(s), or smooth muscle cell(s).

80. The method of any one of the preceding embodiments, wherein the nucleic acid payload comprises a therapeutic transgene.

81. The method of any one of the preceding embodiments, wherein the therapeutic transgene comprises: GLP-1, INS, Reg3g, MafA, PDX-1, NUEROG3, NGN3, DRYK, DYRKIA, DYRK1B, Factor VIII, Factor IX, PKD1, PKD2, COL4A3, COL4A4, COL4A5, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS, or combinations thereof.

82. The method of any one of the preceding embodiments, wherein the nucleic acid payload comprises a therapeutic transgene coupled to a promoter sequence comprising: ApoE promoter, CAG promoter, AAT promoter, or combinations thereof.

83. The method of any one of the preceding embodiments, wherein the nucleic acid payload and the plurality of microbubbles are administered in a volumetric ratio of at least 1 part nucleic acid payload solution to 2 parts microbubble solution, wherein there are at least 2 parts microbubble solution to nucleic acid payload solution.

84. The method of any one of the preceding embodiments, wherein the nucleic acid payload and the plurality of microbubbles are administered in a volumetric ratio of at least 1 part nucleic acid payload solution to 4 parts microbubble solution, wherein there are at least 4 parts microbubble solution to nucleic acid payload solution.

85. The method of any one of the preceding embodiments, wherein the plurality of microbubbles comprises a protein stabilized shell.

86. The method of any one of the preceding embodiments, wherein the plurality of microbubbles comprises a lipid stabilized shell.

87. The method of any one of the preceding embodiments, wherein delivering the nucleic acid to the target cell of the subject increases or decreases expression of a gene in the target cell.

88. A method of delivering a nucleic acid to a target cell of a subject, the method comprising:
providing a treatment session to the subject, wherein the treatment session comprises:
administering a payload comprising a therapeutic transgene greater than about 4.7 kb in length to the subject;
administering a plurality of microbubbles to the subject; and
administering ultrasound energy to the subject in proximity to the target cell(s); and
repeating the treatment session at least once more than 6 hours after but within 10 days after initiation of a first treatment session.

89. The method of embodiment 88, wherein the therapeutic transgene comprises a sequence encoding Factor VIII (FVIII).

90. The method of embodiment 88, wherein the therapeutic transgene comprises a sequence encoding Factor IX (FIX).

91. The method of embodiment 88, wherein the therapeutic transgene comprises a sequence encoding COL4A3.

92. The method of embodiment 88, wherein the therapeutic transgene comprises a sequence encoding COL4A4.

93. The method of embodiment 88, wherein the therapeutic transgene comprises a sequence encoding COL4A5.

94 The method of embodiment 88, wherein the therapeutic transgene comprises a sequence encoding PKD1.

95 The method of embodiment 88, wherein the therapeutic transgene comprises a sequence encoding PKD2.

96. The method of embodiment 88, wherein delivering the nucleic acid to the target cell of the subject increases or decreases expression of a gene in the target cell.

97. A method of delivering a nucleic acid payload to a target cell of a subject, comprising:
administering intravenously through a peripheral vein a first dose of i) the nucleic acid payload, and ii) a plurality of sonoactive microstructures to the subject;
administering ultrasound energy transcutaneously to the subject in proximity to target cell(s) at a first location in the target tissue; and
administering ultrasound energy transcutaneously to the subject in proximity to target cell(s) at a second location in the target tissue.

98. The method of embodiment 97, wherein the method further comprises administering a second dose of i) the payload, and ii) the plurality of sonoactive microstructures to the subject.

99 The method of any one of the preceding embodiments, wherein the method further comprises administering ultrasound energy transcutaneously to the subject in proximity to target cell(s) at a third location in the target tissue.

100. The method of any one of the preceding embodiments, wherein the method further comprises administering intravenously through a peripheral vein a third dose of i) the nucleic acid payload, and ii) the plurality of sonoactive microstructures to the subject.

101. The method of any one of the preceding embodiments, wherein any of the first, the second, or optionally the third dose are administered over a first time period.

102. The method of embodiment 101, wherein the first time period is up to 15, 30, 45, or 60 seconds.

103. The method of any one of the preceding embodiments, wherein the first, the second, and optionally the third dose are administered within 6 hours of one another.

104. The method of any one of the preceding embodiments, wherein the first, the second, and optionally the third dose are administered within 1 hour of one another.

105. The method of embodiment 97, wherein the method increases expression of a nucleic acid or amino acid sequence encoded by the nucleic acid payload by at least 5%, 10%, 20%, 30%, 40%, 50%, or more, after 24 hours as compared to administration of a single dose.

106. The method of any one of the preceding embodiments, wherein the method comprises administering an effective amount of ultrasound energy.

107. The method of any one of the preceding embodiments, wherein the effective amount of ultrasound energy is sufficient to disrupt the sonoactive microstructures.

108. The method of any one of the preceding embodiments, wherein the effective amount of ultrasound energy is sufficient to allow the nucleic acid payload to enter the target cell.

109. The method of any one of the preceding embodiments, wherein the effective amount of ultrasound energy is sufficient to allow the nucleic acid payload to enter the target cell, as demonstrated by expression of the nucleic acid or amino acid sequence encoded by the nucleic acid payload.

110. The method of any one of the preceding embodiments, wherein the effective amount of ultrasound energy comprises applying ultrasound energy at a mechanical index ranging from 0.05 to 1.8.

111. The method of any one of the preceding embodiments, wherein the effective amount of ultrasound energy comprises applying ultrasound energy for up to 30, 60, 90, 120, 150, 180, 240, 300, or 360 seconds.

112. The method of any one of the preceding embodiments, wherein the administering ultrasound energy comprises continuously applying ultrasound energy for the duration of an administration period.

113. The method of any one of the preceding embodiments, wherein continuously applying ultrasound energy comprises applying ultrasound energy at a first mechanical index for a first duration, applying ultrasound energy at a second mechanical index for a second duration, and re-applying ultrasound energy at the first mechanical index without ceasing application of ultrasound energy for the duration of the continuous application of ultrasound energy.

114. The method of any one of the preceding embodiments, wherein administering the effective amount of ultrasound energy comprises administering a plurality of ultrasound flashes several seconds apart from one another.

115. The method any one of the preceding embodiments, wherein the plurality of ultrasound flashes are administered one or more milliseconds apart from one another.

116. The method of any one of the preceding embodiments, wherein the plurality of ultrasound flashes are administered one or more seconds apart from one another.

117. The method of any one of the preceding embodiments, wherein the administering ultrasound energy comprises administering a plurality of ultrasound flashes in which the mechanical index is increased, with each of the plurality of ultrasound flashes administered within 0.9 us to about 2.5 us apart from one another.

118. The method of any one of the preceding embodiments, wherein the administering ultrasound energy comprises administering a plurality of ultrasound flashes in which the mechanical index is increased, with each of the plurality of ultrasound flashes administered within 0.9 us to about 2.5 us apart from one another over about 0.5 s.

119. The method of any one of the preceding embodiments, wherein the administering ultrasound energy of comprises administering a plurality of 10 ultrasound flashes in which the mechanical index is increased, with each of the plurality of 10 ultrasound flashes administered within 2.2 us apart from one another over about 0.5 s.

120. The method of any one of the preceding embodiments, wherein the ultrasound flash is a change in the application of the ultrasound energy from the first mechanical index to the second mechanical index, and reapplication of the ultrasound energy at the first mechanical index from the second mechanical index within a time period.

121. The method of any one of the preceding embodiments, wherein administering the effective amount of ultrasound energy comprises administering at least 5 ultrasound flashes at a second mechanical index which is increased relative to a first mechanical index less than 10 seconds apart from one another.

122. The method of any one of the preceding embodiments, wherein the expression of the nucleic acid payload in the target tissue is maintained for at least 5 days as compared to a baseline expression level.

123. The method of any one of the preceding embodiments, wherein the expression of the nucleic acid payload in the target tissue is maintained for at least 7 days as compared to a baseline expression level.

124. The method of any one of the preceding embodiments, wherein the expression of the nucleic acid payload in the target tissue is maintained for at least 2 weeks.

125. The method of any one of the preceding embodiments, wherein the target tissue is an organ.

126. The method of any one of the preceding embodiments, wherein the organ is a liver.

127. The method of any one of the preceding embodiments, wherein the first location is a first lobe of the liver and the second location is a second lobe of the liver.

128. The method of any one of the preceding embodiments, wherein the first and the second location are different locations of the same lobe of the liver.

129. The method of any one of the preceding embodiments wherein the organ is a kidney.

130. The method of any one of the preceding embodiments, wherein the first location is in a first region of the kidney and the second location is in a second region of the kidney.

131. The method of any one of the preceding embodiments, wherein the first and the second location are different locations of the same region of the kidney.

132. The method of any one of the preceding embodiments, wherein the organ is a pancreas.

133. The method of any one of the preceding embodiments, wherein the first location is in a first region of the pancreas and the second location is in a second region of the pancreas.

134. The method of any one of the preceding embodiments, wherein the first and the second location are different locations of the same region of the pancreas.

135. The method of any one of the preceding embodiments, wherein the organ is a heart.

136. The method of any one of the preceding embodiments, wherein the target tissue is skeletal muscle.

137. The method of any one of the preceding embodiments, wherein the nucleic acid payload is a nanoplasmid.

138. The method of any one of the preceding embodiments, wherein the nucleic acid payload comprises a therapeutic transgene.

139. The method of any one of the preceding embodiments, wherein the therapeutic transgene comprises GLP-1, INS, Reg3g, MafA, PDX-1, NUEROG3, NGN3, DRYK, DYRKIA, DYRK1B, Factor VIII, Factor IX, PKD1, PKD2, COL4A3, COL4A4, COL4A5, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS, or combinations thereof.

140. The method of any one of the preceding embodiments, wherein the nucleic acid payload comprises a therapeutic transgene operably linked to a promoter sequence.

141. The method of any one of the preceding embodiments, wherein the promoter sequence a promoter sequence other than a CMV promoter or a ubiquitin C promoter.

142. The method of any one of the preceding embodiments, wherein the promoter sequence comprises an ApoE promoter, a CAG promoter, an AAT promotor, or combinations thereof.

143. The method of any one of the preceding embodiments, wherein the microbubble comprises a protein stabilized shell.

144. The method of any one of the preceding embodiments, wherein the protein stabilized shell comprises albumin.

145. The method of any one of the preceding embodiments, wherein the microbubble comprises perflutren gas.

146. The method of any one of the preceding embodiments, wherein the sonoactive microstructures are administered at a concentration of at least 5*10\hat{ }8 sonoactive microstructures/mL.

147. The method of any one of the preceding embodiments, wherein the sonoactive microstructures are administered at a concentration between about 5*10\hat{ }8 and about 8*10\hat{ }8 sonoactive microstructures/mL.

148. The method of any one of the preceding embodiments, wherein the plurality of sonoactive microstructures comprise Optison sonoactive microstructures.

149. The method of any one of the preceding embodiments, wherein the sonoactive microstructures comprise a lipid stabilized shell.

150. The method of any one of the preceding embodiments, wherein a concentration of the sonoactive microstructures administered is up to about 1.2*10\hat{ }10 sonoactive microstructures/mL.

151. The method of any one of the preceding embodiments, wherein the method does not result in a substantial elevation of inflammatory biomarker(s).

152. The method of any one of the preceding embodiments, wherein a level of an inflammatory biomarker(s) remains within 20% of a baseline level of the inflammatory biomarker(s) after performing the method.

153. The method of any one of the preceding embodiments, wherein the biomarker(s) is one or more of ALT, AST, and/or IL6.

154. The method of any one of the preceding embodiments, wherein the nucleic acid payload and the plurality of sonoactive microstructures are administered in a volumetric ratio of at least 1 part nucleic acid payload solution to 4 parts microbubble solution.

155. The method of any one of the preceding embodiments, wherein delivering the nucleic acid to the target cell of the subject increases or decreases expression of an endogenous gene in the target cell.

156. A method of delivering a payload into one or more target cells, the method comprising:
administering intravenously through a peripheral vein a first dose of i) a payload comprising a therapeutic transgene, and ii) a plurality of sonoactive microstructures to the subject;
administering ultrasound energy transcutaneously to the subject in proximity to target cell(s) at a first location in the target tissue; and
administering ultrasound energy transcutaneously to the subject in proximity to target cell(s) at a second location in the target tissue.

157. The method of embodiment 156, wherein the therapeutic transgene comprises a sequence encoding FVIII.

158. The method of embodiment 156, wherein the therapeutic transgene comprises a sequence encoding COL4A3.

159. The method of embodiment 156, wherein the therapeutic transgene comprises a sequence encoding COL4A4.

160. The method of embodiment 156, wherein the therapeutic transgene comprises a sequence encoding COL4A5.

161. The method of embodiment 156, wherein the therapeutic transgene comprises a sequence encoding PKD1.

162. The method of embodiment 156, wherein the therapeutic transgene comprises a sequence encoding PKD2.

163. The method of any one of the preceding embodiments, wherein administering the ultrasound energy proximal to the sonoactive microstructures increases microvascular perfusion of a target tissue.

164. The method of any one of the preceding embodiments, wherein administering the ultrasound energy at the second location in the target tissue increases microvascular perfusion of a target tissue.

165. The method of any one of the preceding embodiments, wherein repeating the sonoporation treatment increases microvascular perfusion of a target tissue.

166. The method of any one of the preceding embodiments, wherein the target cell is in a target tissue exhibiting cystic pathology.

167. The method of embodiments 163-165, wherein the target cell is in a target tissue exhibiting cystic pathology, wherein delivery of the nucleic acid to the target cell in the tissue exhibiting cystic pathology is increased.

168. The method of any one of the preceding embodiments, wherein target cell is comprised by a subject having polycystic kidney disease, wherein the therapeutic transgene comprises a sequence encoding PKD1 or PDK2, wherein delivery of the therapeutic transgene to the target cell is increased, thereby treating polycystic kidney disease.

169. A method of delivering a nucleic acid payload to a target cell(s) of a subject, the method comprising:
applying a first treatment session to the subject, the first treatment session comprising:
   administering i) the nucleic acid payload, and ii) a plurality of sonoactive agents to the subject;
   administering ultrasound energy to target cell(s) of the subject at a first location in a target tissue; and
   administering ultrasound energy to target cell(s) of the subject at a second location, in the target tissue, wherein the first location and the second location are different; and
applying a second treatment session to the subject after the first treatment session, the second treatment session comprising:
   administering i) the nucleic acid payload, and ii) a plurality of sonoactive agents to the subject;
   administering ultrasound energy to target cell(s) of the subject at a third location in the target tissue; and
   administering ultrasound energy to target cell(s) of the subject at a fourth location, in the target tissue wherein the third location and the fourth location are different.

170. The method of embodiment 169, further comprising:
applying a subsequent treatment session after the second treatment session to the subject, the subsequent treatment session comprising:
   administering i) the nucleic acid payload, and ii) the plurality of sonoactive agents to the subject;
   administering ultrasound energy to target cell(s) of the subject at a fifth location in the target tissue; and
   administering ultrasound energy to target cell(s) of the subject at a sixth location in the target tissue, wherein the fifth location and the sixth location are different.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method of delivering a nucleic acid payload to a target cell(s) of a subject, the method comprising:
applying a first treatment session to the subject, the first treatment session comprising:
administering an amount of a first therapeutic composition comprising: i) the nucleic acid payload, and ii) a plurality of sonoactive agents to the subject;
administering ultrasound energy to target cell(s) of the subject at a first location in a target tissue; and
administering ultrasound energy to target cell(s) of the subject at a second location, in the target tissue, wherein the first location and the second location are different; and
applying a second treatment session to the subject after the first treatment session, the second treatment session comprising:
administering an amount of a second therapeutic composition comprising: i) the nucleic acid payload, and ii) a plurality of sonoactive agents to the subject;
administering ultrasound energy to target cell(s) of the subject at a third location in the target tissue; and
administering ultrasound energy to target cell(s) of the subject at a fourth location, in the target tissue wherein the third location and the fourth location are different.

2. The method of claim 1, further comprising:
applying a subsequent treatment session after the second treatment session to the subject, the subsequent treatment session comprising:
administering an amount of the first or second therapeutic composition comprising: i) the nucleic acid payload, and ii) the plurality of sonoactive agents to the subject;
administering ultrasound energy to target cell(s) of the subject at a fifth location in the target tissue; and
administering ultrasound energy to target cell(s) of the subject at a sixth location in the target tissue, wherein the fifth location and the sixth location are different.

3. The method of any one of the preceding claims, further comprising, in the first treatment session, administering ultrasound energy to target cell(s) of the subject at a subsequent location in the target tissue after administering ultrasound energy to the second location.

4. The method of any one of the preceding claims, further comprising, in the second treatment session, administering ultrasound energy to target cell(s) of the subject at a subsequent location in the target tissue after administering ultrasound energy to the fourth location.

5. The method of claim 2, further comprising, in the subsequent treatment session, administering ultrasound energy to target cell(s) of the subject at a subsequent location in the target tissue after administering ultrasound energy to the sixth location.

6. The method of any one of the preceding claims, wherein the second treatment session is more than 6 hours after but within 10 days of the first treatment session.

7. The method of any one of the preceding claims, wherein the second treatment session is more than 21 days after the first treatment session.

8. The method of any one of the preceding claims, wherein administering of the first therapeutic composition and/or the second therapeutic composition occurs intravenously through a peripheral vein.

9. The method of any one of the preceding claims, wherein the nucleic acid payload comprises a therapeutic transgene greater than 4.7 kbp in length, and/or an expression cassette greater than 4.7 kbp in length.

10. The method of claim 9, wherein the therapeutic transgene comprises FVIII, COL4A5, or PKD1, GLP-1, INS, Reg3g, MafA, PDX-1, NUEROG3, NGN3, DRYK, DYRKIA, DYRKIB, Factor VIII, Factor IX, PKD2, COL4A3, COL4A4, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS, or combinations thereof.

11. The method of any one of the preceding claims, wherein administering the amount of the first therapeutic composition comprises administering to the subject, in the first treatment session, a first dose of the first therapeutic composition and a second dose of the first therapeutic composition.

12. The method of claim 11, wherein, in the first treatment session, administering ultrasound energy to the subject in the second location in the target tissue occurs during or after the administering the second dose of the first therapeutic composition.

13. The method of claim 11, wherein administering the amount of the first therapeutic composition in the first treatment session comprises administering at least a third dose of the first therapeutic composition.

14. The method of claim 13, further comprising, in the first treatment session, administering ultrasound energy to the subject at the subsequent location during or following the administering of the third dose.

15. The method of any one of the preceding claims, wherein administering the amount of the second therapeutic composition in the second treatment session comprises administering to the subject a first dose of the second therapeutic composition and a second dose of the second therapeutic composition.

16. The method of claim 15, wherein administering ultrasound energy, in the second treatment session, to the subject at the third location in the target tissue occurs during or after the administering the second dose of the second therapeutic composition.

17. The method of claim 15, wherein administering the amount of the second therapeutic composition in the second treatment session comprises administering at least a third dose of the second therapeutic composition.

18. The method of claim 17, comprising administering, in the second treatment session, ultrasound energy to the subject at the subsequent location during or following the administering of the third dose of the second therapeutic composition.

19. The method of any one of claims 11-18, wherein each dose of the first therapeutic composition or the second therapeutic composition is administered as an intravenous injection.

20. The method of claim 19, wherein the intravenous injection is administered over a discrete time period.

21. The method of claim 20, wherein the discrete time period is no more than 60, 120, or 180 seconds.

22. The method of any one of the preceding claims, wherein the first location in the target tissue and the third location in the target tissue are a same location.

23. The method of any one claim 1-21, wherein the first location in the target tissue and the third location in the target tissue are different.

24. The method of claim 3, wherein, the first location in the target tissue and the subsequent location in the target tissue are a same location, in the first treatment session.

25. The method of claim 3, wherein the first location in the target tissue and the subsequent location in the target tissue are the different, in the first treatment session.

26. The method of any one of the preceding claims, wherein the second location in the target tissue and the fourth location in the target tissue are a same location.

27. The method any one of claim 1-25, wherein the second location in the target tissue and the fourth location in the target tissue are different.

28. The method of claim 4, wherein, in the second treatment session, in the target tissue and the subsequent location in the target tissue are a same location, in the second treatment session.

29. The method of claim 4, wherein, in the second treatment session, the second location in the target tissue and the subsequent location in the target tissue are different.

30. The method of claim 2, wherein the fifth location is a same location as any one of the first to fourth locations.

31. The method of claim 2, wherein the sixth is a same location as any one of the first to fourth locations.

32. The method of any one of the preceding claims, wherein the ultrasound energy is administered at an MI of at least 1.6, 2.1, or 2.3.

33. The method of any one of the preceding claims, wherein the sonoactive agents comprise a protein stabilized shell, a lipid stabilized shell, a perflutran gas core, an SF6 gas core, or combinations thereof.

34. The method of any one of the preceding claims, wherein the ultrasound energy is administered transcutaneously.

35. The method of any one of the preceding claims, wherein the target cell(s) and/or the target tissue are in a liver.

36. The method of claim 35, wherein the first location is a first lobe of the liver and the second location or subsequent location is a second lobe or a subsequent lobe of the liver.

37. The method of claim 36, wherein the first lobe of the liver is a right lobe, wherein the second lobe is a left lobe, and wherein the subsequent lobe is one or both of a caudate lobe or a quadrate lobe.

38. The method of claim 35, wherein the nucleic acid payload comprises a therapeutic transgene, wherein target cell is a hepatocyte, and wherein at least 50% of cells expressing the therapeutic transgene in the liver are hepatocytes.

39. The method of any one of claims 1-34, wherein the target cell(s) and/or the target tissue are in a kidney.

40. The method of claim 39, wherein the first location is in a first region of the kidney and the second location is in a second region of the kidney.

41. The method of claim 39, wherein expression of the nucleic acid payload is induced in multiple cell types in the kidney.

42. The method of claim 39, wherein a transgene encoded by the nucleic acid payload is expressed in cells in all regions of the kidney.

43. The method of claim 39, wherein a transgene encoded by the nucleic acid payload is expressed in cells across four non-overlapping spatial regions of the kidney, the four non-overlapping spatial regions of the kidney defining an entire kidney.

44. The method of any one of the preceding claims, wherein a distance between the first location in the target tissue and the second location or subsequent location in the target tissue is at least 25% of a maximum distance of a major axis of an organ comprising the target tissue.

45. The method of any one of the preceding claims, wherein a distance between the third location in the target tissue and the fourth location or subsequent location in the target tissue is at least 25% of a maximum distance of major axis of an organ comprising the target tissue.

46. The method of any one of the preceding claims, wherein a distance between the first location in the target tissue and the second location or subsequent location in the target tissue is at least 1, 2, or 3 cm.

47. The method of any one of the preceding claims, wherein a distance between the third location in the target tissue and the fourth location or subsequent location in the target tissue is at least 1, 2, or 3 cm.

48. The method of any one of the preceding claims, wherein a distance between the first location in the target tissue and the second location or subsequent location in the target tissue is at least a diameter of an ultrasound focal beam.

49. The method of any one of the preceding claims, wherein a distance between the third location in the target tissue and the fourth location or subsequent location in the target tissue is at least a diameter of an ultrasound focal beam.

50. The method of any one of the preceding claims, wherein administering the ultrasound energy at the second location or subsequent location in the target tissue increases microvascular perfusion of the target tissue.

51. The method of any one of the preceding claims, wherein the target tissue exhibits a cystic pathology.

52. The method of claim 51, wherein delivery and/or expression of the nucleic acid payload to the target cell(s) in the target tissue exhibiting the cystic pathology is increased.

53. The method of any one of the preceding claims, wherein the first therapeutic composition and the second therapeutic composition are a same therapeutic composition.

54. The method of any one of the preceding claims, wherein the first therapeutic composition and the second therapeutic composition comprise different dosages of the nucleic acid payload.

55. The method of any one of the preceding claims, wherein the first therapeutic composition and the second therapeutic composition comprise different dosages of sonoactive agents, or different sonoactive agents.

56. The method of any one of the preceding claims, wherein administering ultrasound energy to the subject at the first location and at the second location comprises moving an ultrasound probe across a surface of the subject's skin from the first location to the second location.

57. The method of any one of the preceding claims, wherein administering the second treatment session increases delivery or expression of the nucleic acid payload by at least 25%, 50%, 75%, 100%, 250%, 300%, 350%, 400%, 450%, or 500%, as compared to a method comprising administration of the first treatment session without the second treatment session.

58. The method of claim 2, wherein administering the subsequent treatment session increases delivery or expression of the nucleic acid payload by at least 100%, 250%, 300%, 350%, 400%, 450%, or 500%, as compared to a method comprising administration of the first treatment session without the second treatment session, or as compared to a method comprising administration of the first treatment session and the second treatment session without administration of the subsequent treatment session.

59. The method of any one of the preceding claims, wherein the first location and the second location are contiguous locations of the target tissue.

60. The method of claim 2, wherein the second location and the subsequent location are contiguous locations of the target tissue.

61. The method of any one of the preceding claims, wherein the third location and the fourth location are contiguous locations of the target tissue.

62. The method of claim 3, wherein the fourth location and the subsequent location are contiguous locations of the target tissue.

63. The method of claim 2, wherein the fifth location or the sixth location are contiguous with any one of the first location to the fourth location.
